(19) United States
(12) Patent Application Publication
(10) Pub. No.: US 2026/0209178 A1
(43) Pub. Date: Jul. 23, 2026

(54) PROCESSES FOR THE PREPARATION OF NLRP3 INHIBITORS

(71) Applicant: Hoffmann-La Roche Inc., Little Falls, NJ (US)

(72) Inventors: Satyender Apuri, Gachibowli (IN); Sreenivasulu Bandaru, Gachibowli (IN); Mallesh Bharatha, Hyderabad (IN); Raghavendra Babu Chiliveri, Gachibowli (IN); Joséphine Eliette Françoise Cinqualbre, Saint-Louis (FR); Paul Fraser, Abingdon (GB); Prathap Ireddy, Gachibowli (IN); Dainis Kaldre, Lörrach (DE); Régis Jean Georges Mondière, Waltenheim (FR); Jetta Palguna, Hyderabad, Telangana (IN); Alfred Stutz, Dottikon (CH); Paolo Tosatti, Binningen (CH)

(73) Assignee: Hoffmann-La Roche Inc., Little Falls, NJ (US)

(21) Appl. No.: 19/142,014

(22) PCT Filed: Dec. 21, 2023
(86) PCT No.: PCT/EP2023/087167

§ 371 Date: Jun. 20, 2025

(30) Foreign Application Priority Data
Dec. 23, 2022 (IN) 202241074900
Feb. 23, 2023 (EP) 23158222.2

(51) Int. Cl. C07D 211/54

(52) U.S. Cl. CPC C07D 211/54

(57) ABSTRACT

The present invention relates to intermediates and processes useful for preparing 1-ethyl-N-((_{1,2,3,5,6,7}-hexahydro-s-indacen-4-yl)carbamoyl)piperidine-4-sulfonamide 5 and salts thereof. The present invention further relates to 1-ethyl-N-((_{1,2,3,5,6,7}-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP₃ inhibition.

### FIELD OF THE INVENTION

The present invention relates to intermediates and processes useful for preparing 1-ethyl-N-((1,2,3,5,6,7-hexahydro-s-indacen-4-yl)carbamoyl)piperidine-4-sulfonamide and salts thereof. The present invention further relates to 1-ethyl-N-((1,2,3,5,6,7-hexahydro-s-indacen-4-yl)carbamoyl)piperidine-4-sulfonamide and salts thereof when prepared by such processes and to associated pharmaceutical compositions and uses for the treatment and prevention of medical disorders and diseases, most especially by NLRP3 inhibition.

### BACKGROUND

1-Ethyl-N-((1,2,3,5,6,7-hexahydro-s-indacen-4-yl)carbamoyl)piperidine-4-sulfonamide is disclosed in WO 2019/008025 A1 as an NLRP3 inhibitor (see Example 6). However, there is a need to provide improved processes for preparing 1-ethyl-N-((1,2,3,5,6,7-hexahydro-s-indacen-4-yl)carbamoyl)piperidine-4-sulfonamide and salts thereof. In particular, there is a need to provide efficient processes that are suitable for large scale synthesis and which, for example, avoid costly chromatographic or high temperature techniques, avoid or minimise the use of expensive or environmentally unfriendly reagents, and/or avoid the generation of hazardous by-products. There is also a need to provide 1-ethyl-N-((1,2,3,5,6,7-hexahydro-s-indacen-4-yl)carbamoyl)piperidine-4-sulfonamide and salts thereof, and intermediates on route to such a compound, in a higher yield and/or with a higher purity compared to prior art processes, especially on a large scale. The present invention solves the aforementioned problems.

### SUMMARY OF THE INVENTION

A first aspect of the invention provides a process of preparing a thiourea adduct (I) or a salt thereof, the process comprising the step of converting a N-protected-4-derivatised piperidine (H) to the thiourea adduct (I) or the salt thereof:

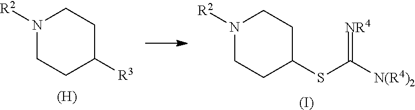

wherein:
   R² is a nitrogen protecting group;
   R³ is a leaving group; and
   each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

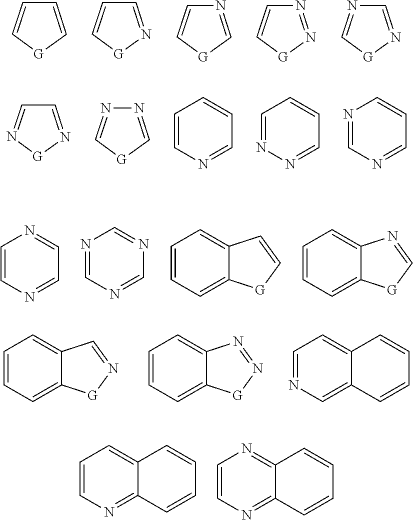

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Similarly, unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

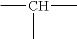

is replaced by

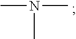

—CH₂— is replaced by —NH—, —O— or —S—;
—CH₃ is replaced by —NH₂, —OH or —SH;
—CH═ is replaced by —N═;
CH₂═ is replaced by NH═, O═ or S═; or
CH≡ is replaced by N≡;
   provided that the resultant group comprises at least one carbon atom. For example, methoxy, dimethylamino and aminoethyl groups are considered to be hydrocarbyl groups including one or more heteroatoms N, O or S in their carbon skeleton.

As used herein, where it is stated that a group such as a hydrocarbyl group is substituted with an oxo (═O) group, it is to be understood that any two hydrogen atoms attached to the same atom may be replaced by a π-bonded ═O substituent, or where the group contains a nitrogen or sulfur atom, the oxidation state of the nitrogen or sulfur atom may be changed so as to permit the attachment of a π-bonded ═O substituent, optionally with the loss of one or more hydrogen atoms from the nitrogen atom, the sulfur atom or a neighbouring atom to allow for charge neutralisation. Thus, for example, —CH₂CHO, —CH₂NO₂ and —CH₂SO₃H are examples of —CH₂CH₃, —CH₂NHOH and —CH₂—S—OH groups respectively substituted with one (—CH₂CHO, —CH₂NO₂) or two (—CH₂SO₃H) oxo groups.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are not to be counted as carbon atoms when calculating the number of carbon atoms in a Cₓ-C_{y} group. For example, a morpholinyl group is to be considered a C₄ heterocyclic group, not a C₆ heterocyclic group.

In one embodiment of the first aspect of the invention, the process comprises the step of contacting the N-protected-4-derivatised piperidine (H) with reagent (I-X):

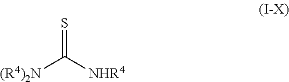

optionally in the presence of a base and/or a solvent, wherein each R⁴ is as defined above.

As stated, R² is a nitrogen protecting group. Suitable nitrogen protecting groups may be identified by reference to e.g. Wuts, ‘Greene's Protective Groups in Organic Synthesis’, 5^{th} Ed., 2014, the contents of which are incorporated herein by reference in their entirety.

In one embodiment of the first aspect of the invention, R² is a nitrogen protecting group that is stable under basic conditions. Typically, R² is also stable under weak nucleophilic conditions, such as on exposure to thiourea. For example, R² may be selected from the group consisting of benzyloxycarbonyl (CBz), 4-methoxy-benzyloxycarbonyl, benzyl, t-butoxycarbonyl (Boc), 2-(4-biphenylyl)-isopropoxycarbonyl (Bpoc), triphenylmethyl (Trt) and 2,2,2-trichloroethoxycarbonyl (Troc) protecting groups.

In one embodiment of the first aspect of the invention, R² is a nitrogen protecting group that may be removed by catalytic hydrogenolysis. Typically, R² is a nitrogen protecting group that is stable under basic conditions, and that may be removed by catalytic hydrogenolysis. More typically, R² is a nitrogen protecting group that is stable under basic and weak nucleophilic conditions, and that may be removed by catalytic hydrogenolysis. For example, R² may be selected from the group consisting of benzyloxycarbonyl (CBz), 4-methoxy-benzyloxycarbonyl, benzyl, 2-(4-biphenylyl)-isopropoxycarbonyl (Bpoc) and triphenylmethyl (Trt) groups.

In a further embodiment of the first aspect of the invention, R² is —CH₂R²⁰ or —COOCH₂R²⁰, wherein R²⁰ is an aryl or heteroaryl group, wherein the aryl or heteroaryl group is monocyclic, bicyclic or tricyclic, wherein the aryl or heteroaryl group may optionally be substituted with one or more substituents independently selected from halo, —CN, —OH, —NO₂, —NH₂, —R²¹, —OR²¹, —NHR²¹, —N(R²¹)₂ or —N(O)(R²¹)₂, wherein each R²¹ is independently selected from a C₁-C₄ alkyl, C₁-C₄ haloalkyl, C₃-C₄ cycloalkyl or C₃-C₄ halocycloalkyl group, or any two R²¹ directly attached to the same nitrogen atom may together form a C₂-C₅ alkylene or C₂-C₅ haloalkylene group, and wherein R²⁰, including any optional substituents, contains from 1 to 20 carbon atoms.

In one embodiment of the first aspect of the invention, R² is —COOCH₂R²⁰.

In one embodiment of the first aspect of the invention, R²⁰ is selected from a phenyl or a monocyclic heteroaryl group, wherein R²⁰ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R²⁰, including any optional substituents, contains from 1 to 12 carbon atoms.

Typically, R²⁰ is a phenyl group, wherein the phenyl group is optionally substituted with one or more fluoro, chloro, —OMe, —OEt, or —NO₂ groups.

More typically, R²⁰ is a phenyl group. For example, R² may be —CH₂Ph or —COOCH₂Ph.

Most typically, R² is —COOCH₂Ph (i.e. a benzyloxycarbonyl (CBz) group).

As stated, R³ is a leaving group. In one embodiment of the first aspect of the invention, R³ is selected from Cl, Br, I, or a sulfonate leaving group such as a toluenesulfonate (tosylate or —OTs), methanesulfonate (mesylate or —OMs), or trifluoromethanesulfonate (triflate or —OTf) leaving group. Typically, R³ is a sulfonate leaving group. Most typically R³ is —OMs.

In one embodiment of the first aspect of the invention, each R⁴ is independently selected from hydrogen or a C₁-C₁₂ alkyl or -L⁴¹-R⁴¹ group, or any two R⁴ may together form a -L⁴²- group, wherein:
each C₁-C₁₂ alkyl group may optionally be halo substituted and may optionally include one, two or three oxygen atoms in its carbon skeleton;
each L⁴¹ is independently selected from a bond or a C₁-C₄ alkylene group, wherein each C₁-C₄ alkylene group may optionally be halo substituted and may optionally include one, two or three oxygen atoms in its carbon skeleton;
each R⁴¹ is independently selected from a C₃-C₇ cycloalkyl, phenyl, napthyl or monocyclic or bicyclic heteroaryl group, wherein any C₃-C₇ cycloalkyl group may optionally include one or two oxygen atoms in its carbon skeleton, any wherein any C₃-C₇ cycloalkyl, phenyl, napthyl or monocyclic or bicyclic heteroaryl group may optionally be substituted with one or more halo and/or one or more R⁴³ groups, provided that each -L⁴¹-R⁴¹ group, including any optional substituents, contains no more than 12 carbon atoms;
each L⁴² is independently selected from a C₂-C₆ alkylene group, wherein any C₂-C₆ alkylene group may optionally include one or two oxygen atoms in its carbon skeleton, and wherein any C₂-C₆ alkylene group may optionally be substituted with one or more halo and/or R⁴³ groups, provided that each -L⁴²- group, including any optional substituents, contains no more than 12 carbon atoms; and
each R⁴³ group is independently selected from a C₁-C₄ alkyl, —O—(C₁-C₄ alkyl), C₁-C₄ haloalkyl, —O—(C₁-C₄ haloalkyl), C₃-C₄ cycloalkyl, —O—(C₃-C₄ cycloalkyl), C₃-C₄ halocycloalkyl or —O—(C₃-C₄ halocycloalkyl) group.

In a further embodiment of the first aspect of the invention, each R⁴ is independently selected from hydrogen or a C₁-C₆ alkyl or C₃-C₆ cycloalkyl group, or any two R⁴ may together form a C₂-C₆ alkylene group, wherein any C₁-C₆ alkyl, C₃-C₆ cycloalkyl or C₂-C₆ alkylene group may optionally be fluoro substituted.

Typically in accordance with the first aspect of the invention, at least one R⁴ is hydrogen. For example, the thiourea adduct (I) in accordance with the first aspect of the invention may have the formula (Ia):

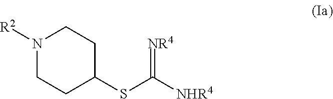

wherein R² and R⁴ are as defined above. Typically in such an embodiment, the thiourea adduct (Ia) or the salt thereof is prepared by contacting the N-protected-4-derivatised piperidine (H) with reagent (I-Xa):

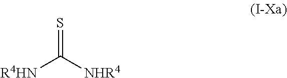

optionally in the presence of a base and/or a solvent, wherein each R⁴ is as defined above.

Most typically in accordance with the first aspect of the invention, each R⁴ is hydrogen. As will be understood, in such an embodiment the thiourea adduct (I) in accordance with the first or the second aspect of the present invention may have the formula (Ib):

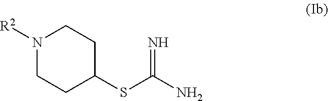

wherein R² is as defined above. Typically in such an embodiment, the thiourea adduct (Ib) or the salt thereof is prepared by contacting the N-protected-4-derivatised piperidine (H) with reagent (I-Xb):

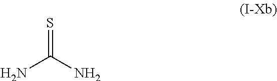

optionally in the presence of a base and/or a solvent.

Typically, the process of the first aspect of the invention is performed in the presence of a solvent.

In one embodiment, the solvent is a polar solvent or a mixture of polar and non-polar solvents. For example, the solvent may comprise one or more polar protic solvents and/or one or more polar aprotic solvents and/or one or more non-polar solvents.

Typically the solvent does not comprise an ester. Typically, the solvent is not halogenated. Suitable polar protic solvents include water and alcohols such as methanol, ethanol, n-propanol, isopropanol, n-butanol, sec-butanol, iso-butanol, tert-butanol and tert-amyl alcohol. Suitable polar aprotic solvents include dimethyl sulfoxide, N,N-dimethylformamide, N,N′-dimethylpropyleneurea, tetrahydrofuran, 1,4-dioxane, acetone, acetonitrile, dichloromethane, hexamethylphosphoramide, nitromethane, propylene carbonate and N-methyl pyrrolidone. Suitable non-polar solvents include pentane, cyclopentane, hexane, cyclohexane, diethyl ether and toluene.

Typically, the solvent is a polar protic solvent or a mixture of a polar protic solvent and a non-polar solvent. Typically in such an embodiment, the polar protic solvent is selected from water or an alcohol, such as methanol, ethanol, n-propanol, isopropanol, n-butanol, sec-butanol, iso-butanol, tert-butanol, tert-amyl alcohol, or any mixture thereof. More typically, the polar protic solvent is selected from an alcohol, such as methanol, ethanol, n-propanol, isopropanol, n-butanol, sec-butanol, iso-butanol, tert-butanol, tert-amyl alcohol, or a mixture thereof. Most typically, the polar protic solvent is n-butanol. Typically, where a non-polar solvent is present, the non-polar solvent is selected from pentane, cyclopentane, hexane, cyclohexane, diethyl ether, toluene, or a mixture thereof. Most typically, the non-polar solvent is toluene.

In an exemplary embodiment of the first aspect of the invention, the solvent is a mixture of n-butanol and toluene.

Where the solvent is a mixture of a polar protic solvent and a non-polar solvent, such as a mixture of n-butanol and toluene, typically the ratio of polar protic solvent to non-polar solvent is >3:1 by volume. More typically, the ratio of polar protic solvent to non-polar solvent is >10:1 by volume. More typically still, the ratio of polar protic solvent to non-polar solvent is >20:1 by volume.

Optionally, the process of the first aspect of the invention is performed in the presence of a base. Typically, the base is a sterically hindered base. For example, the base may be a tertiary alkoxide base such as a tertiary butoxide base, or a tertiary amine such as N,N-diisopropylethylamine (DIPEA), trimethylamine, triethylamine (TEA), tripropylamine or tributylamine. Most typically, the base if present is triethylamine.

Optionally, the process of the first aspect of the invention is performed in the presence of a nucleophilic catalyst. For example an iodide source such as NaI may be used. It is noted however that the process of the first aspect of the invention is able to proceed in the absence of a nucleophilic catalyst, which may be advantageous since it allows for a more facile work-up procedure. Accordingly, in one embodiment of the first aspect of the invention a nucleophilic catalyst is not added to the reaction mixture.

In an exemplary embodiment of the first aspect of the invention, the process comprises the step of contacting benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′) or a salt thereof:

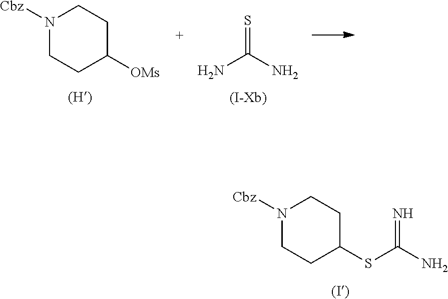

Typically in such an embodiment, the solvent is n-butanol or a mixture of n-butanol and toluene.

Typically in accordance with the first aspect of the invention, the N-protected-4-derivatised piperidine (H) or (H′) is combined with the reaction mixture in non-salt form.

Typically in accordance with the first aspect of the invention, the reagent (I-X), (I-Xa) or (I-Xb) is combined with the reaction mixture in non-salt form.

Typically in accordance with the first aspect of the invention, the thiourea adduct (I) or (I′) is obtained in salt form. More typically, the thiourea adduct (I) or (I′) is obtained as a sulfonic acid addition salt. Most typically, the thiourea adduct (I) or (I′) is obtained as a methanesulfonic acid salt.

In one embodiment of the first aspect of the invention, the step of converting the N-protected-4-derivatised piperidine (H) or (H′), to the thiourea adduct (I) or (I′), or the salt thereof, is carried out at a temperature in the range from 20 to 150° C. Typically, the step is carried out at a temperature in the range from 75 to 125° C., and more typically in the range from 90 to 110° C.

Typically in accordance with the first aspect of the invention, the N-protected-4-derivatised piperidine (H) or (H′) is present in or added to the solvent at an initial concentration of from 0.01 to 10 mol/L relative to the total volume of solvent used in the reaction mixture. More typically, the N-protected-4-derivatised piperidine (H) or (H′) is present in or added to the solvent at an initial concentration of from 0.5 to 2.0 mol/L. Most typically the N-protected-4-derivatised piperidine (H) or (H′) is present in or added to the solvent at an initial concentration of from 0.8 to 1.2 mol/L.

Typically, the process of the first aspect of the invention uses from 0.9 to 3.0 molar equivalents of the reagent (I-X), (I-Xa) or (I-Xb), relative to the initial amount of the N-protected-4-derivatised piperidine (H) or (H′). More typically, the process uses from 0.95 to 1.5 molar equivalents of the reagent (I-X), (I-Xa) or (I-Xb). Most typically, the process uses from 1.0 to 1.2 molar equivalents of the reagent (I-X), (I-Xa) or (I-Xb). Typically, where the process of the first aspect of the invention employs a base, the process uses from 0.9 to 3.0 molar equivalents of the base, relative to the initial amount of the N-protected-4-derivatised piperidine (H) or (H′). More typically, the process uses from 0.95 to 1.5 molar equivalents of the base. Most typically, the process uses from 1.0 to 1.2 molar equivalents of the base.

Typically, where the process of the first aspect of the invention employs a nucleophilic catalyst, the process uses from 0.01 to 0.5 molar equivalents of the nucleophilic catalyst, relative to the initial amount of the N-protected-4-derivatised piperidine (H) or (H′). More typically, the process uses from 0.02 to 0.3 molar equivalents of the nucleophilic catalyst. Most typically, the process uses from 0.05 to 0.15 molar equivalents of the nucleophilic catalyst.

In one embodiment of the first aspect of the invention, the process comprises the steps of:
(1) providing a solution of the N-protected-4-derivatised piperidine (H) or (H′) in a non-polar solvent such as toluene;
(2) adding to the solution of step (1) a polar protic solvent such as n-butanol, and the reagent (I-X), (I-Xa) or (I-Xb), to form a mixture;
(3) optionally removing a portion of the solvent from step (2) by distillation, e.g. under reduced pressure;
(4) optionally adding a further portion of the polar protic solvent; and
(5) heating the mixture, typically to a temperature in the range from 75 to 125° C., and more typically in the range from 90 to 110° C.

Typically in accordance with the first aspect of the invention, steps (3) and (4) are not optional.

In one embodiment of the first aspect of the invention, the thiourea adduct (I) or (I′), or the salt thereof, is isolated from the reaction mixture by crystallisation or precipitation. For example, the reaction mixture may be cooled, optionally with seeding, to form a slurry of the solid product, which may then be collected by filtration. The collected solid may then be washed, e.g. with an alcohol such as isopropanol, and optionally dried under vacuum. For example, the process of the first aspect of the invention comprising steps (1)-(5) above, may further comprise the steps of:
(6) seeding the mixture with crystals of the thiourea adduct (I) or (I′), or the salt thereof;
(7) maintaining the seeded mixture at an elevated temperature, typically at a temperature in the range from 75 to 125° C., and more typically in the range from 90 to 110° C., for a period of 1 hour or more;
(8) cooling the seeded mixture, typically to a temperature in the range from 0 to 50° C., and more typically in the range from 15 to 30° C., to form a slurry comprising the thiourea adduct (I) or (I′), or the salt thereof, as a solid;
(9) collecting the solid by filtration;
(10) optionally washing the collected solid with a solvent, e.g. with an alcohol such as isopropanol; and
(11) optionally drying the collected solid under vacuum.

Typically in accordance with the first aspect of the invention, steps (10) and (11) are not optional.

In one embodiment of the first aspect of the invention, the N-protected-4-derivatised piperidine (H) is obtained by a process comprising the step of:
(ii) converting a N-protected-4-hydroxy piperidine (G) to the N-protected-4-derivatised piperidine (H):

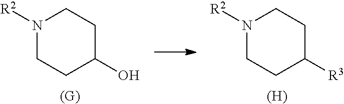

wherein R² is a nitrogen protecting group and R³¹ is a leaving group.

In other words, in one embodiment of the first aspect of the invention, there is provided a process of preparing a thiourea adduct (I) or a salt thereof, the process comprising at least the steps of:
(ii) converting a N-protected-4-hydroxy piperidine (G) to a N-protected-4-derivatised piperidine (H):

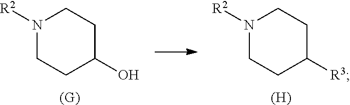

and
(iii) converting the N-protected-4-derivatised piperidine (H) to the thiourea adduct (I) or the salt thereof:

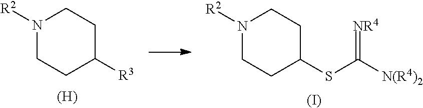

wherein:
R² is a nitrogen protecting group;
R³ is a leaving group; and
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each 25 R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

As will be appreciated, where the first aspect of the invention comprises steps (ii) and (iii), R² and R³ are the same in each step. All optional, typical and exemplary embodiments as described above in relation to the first aspect of the invention apply equally to step (iii).

In one embodiment of the first aspect of the invention, the reaction step (ii) comprises contacting the N-protected-4-hydroxy piperidine (G) with SOCl₂, SOBr₂, or a mixture of Ph₃P and Cl₂ or Br₂, to form the N-protected-4-derivatised piperidine (H), wherein R³ is as appropriate a Cl or Br leaving group.

In another embodiment of the first aspect of the invention, the reaction step (ii) comprises contacting the N-protected-4-hydroxy piperidine (G) with a sulfonyl halide or a sulfonyl anhydride to form the N-protected-4-derivatised piperidine (H), wherein R³ is a sulfonate leaving group.

As will be appreciated, the sulfonyl halide or sulfonyl anhydride used will correspond to the sulfonate leaving group of R³. For example, where R³ is a tosylate leaving group a tosyl halide or tosyl anhydride will be used. Similarly, where R³ is a mesylate leaving group a mesyl halide or mesyl anhydride will be used, and where R³ is a triflate leaving group a triflic halide or triflic anhydride will be used.

Typically, a sulfonyl halide is used. In one embodiment, the sulfonyl halide is selected from a sulfonyl chloride, a sulfonyl bromide, or a sulfonyl iodide. Typically, the sulfonyl halide is a sulfonyl chloride or a sulfonyl bromide. More typically, the sulfonyl halide is a sulfonyl chloride.

In a typical embodiment of the first aspect of the invention, the reaction step (ii) comprises contacting the N-protected-4-hydroxy piperidine (G) with a mesyl halide or mesyl anyhdride to form the N-protected-4-derivatised piperidine (H), wherein R³ is a mesylate leaving group. Most typically in such an embodiment the reaction step (ii) comprises contacting the N-protected-4-hydroxy piperidine (G) with mesyl chloride.

Typically, the reaction step (ii) is carried out in the presence of a solvent. Typically the solvent is aprotic. Typically, the reaction step (ii) comprises contacting the N-protected-4-hydroxy piperidine (G) with a sulfonyl halide or a sulfonyl anhydride in an aprotic solvent.

In one embodiment, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. For example, the solvent may be selected from dimethyl sulfoxide, tetrahydrofuran, 1,4-dioxane, acetonitrile, dichloromethane, hexamethylphosphoramide, nitromethane, or a mixture thereof. More typically still, the solvent does not comprise a carbonyl, C═N or C≡N group. For example, the solvent may be selected from dimethyl sulfoxide, tetrahydrofuran, 1,4-dioxane, dichloromethane, hexamethylphosphoramide, nitromethane, or a mixture thereof. In one embodiment, the solvent is dichloromethane.

In another embodiment, the solvent is a non-polar solvent, such as pentane, cyclopentane, hexane, cyclohexane, diethyl ether, toluene, or a mixture thereof. Typically, the non-polar solvent is not halogenated. Most typically, the non-polar solvent is toluene.

In one embodiment of the first aspect of the invention, the reaction step (ii) is carried out in the presence of a base. Typically, the reaction step (ii) comprises contacting the N-protected-4-hydroxy piperidine (G) with a sulfonyl halide or a sulfonyl anhydride in the presence of a base. Typically, the base is a sterically hindered base. For example, the base may be a tertiary amine such as N,N-diisopropylethylamine (DIPEA), trimethylamine, triethylamine (TEA), tripropylamine or tributylamine. Most typically, the base is triethylamine (TEA).

In an exemplary embodiment of the first aspect of the invention, there is provided a process of preparing benzyl 4-(carbamimidoylthio)-piperidine-1-carboxylate (I′) or a salt thereof, the process comprising the steps of:
(ii) contacting N-carboxybenzyl-4-hydroxy piperidine (G′) with mesyl chloride to obtain benzyl 4-((methylsulfonyl)oxy)piperidine-1-carboxylate (H′):

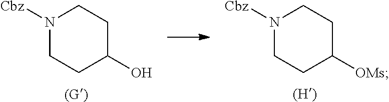

and
(iii) contacting the benzyl 4-((methylsulfonyl)oxy)piperidine-1-carboxylate (H′) with reagent (I-Xb) in a solvent to obtain the benzyl 4-(carbamimidoylthio)-piperidine-1-carboxylate (I′) or the salt thereof:

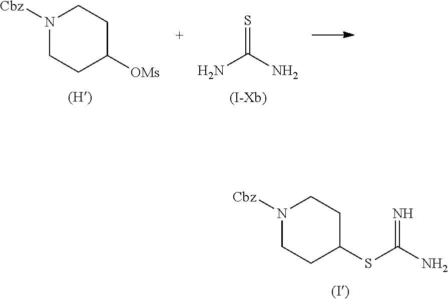

Typically in such an embodiment, in step (ii) the N-carboxybenzyl-4-hydroxy piperidine (G′) is contacted with the mesyl chloride in the presence of a tertiary amine base such as triethylamine and an aprotic solvent. Typically the aprotic solvent is a non-polar solvent such as toluene.

Typically, where the process of the first aspect of the invention comprises step (ii), the N-protected-4-hydroxy piperidine (G) or (G′) is combined with the reaction mixture of step (ii) in non-salt form.

Typically, where the process of the first aspect of the invention comprises step (ii), the N-protected-4-derivatised piperidine (H) or (H′) is obtained in step (ii) in non-salt form.

In one embodiment of the first aspect of the invention, the N-protected-4-derivatised piperidine (H) or (H′) is not isolated between steps (ii) and (iii).

In another embodiment of the first aspect of the invention, at the end of the reaction the process of step (ii) further comprises the work-up step of subjecting the reaction mixture to an aqueous wash such that the N-protected-4-derivatised piperidine (H) or (H′) is retained in the organic phase. Typically in such an embodiment, the reaction solvent is a non-polar solvent such as toluene. In one embodiment, the solvent is removed from the organic phase to afford the N-protected-4-derivatised piperidine (H) or (H′). Alternately, the N-protected-4-derivatised piperidine (H) or (H′) in the organic phase may be used directly in step (iii). Optionally in such an embodiment, a portion of the solvent, e.g. about 50-75% by volume, is removed from the organic phase, e.g. by distillation under reduced pressure. The remainder of the organic solvent comprising the N-protected-4-derivatised piperidine (H) or (H′) may then be used directly in step (iii). As will be appreciated, in such an embodiment the solvent of the organic phase provides all or part of the solvent used in the reaction of step (iii).

In one embodiment of the first aspect of the invention, the N-protected-4-hydroxy piperidine (G) is obtained by a process comprising the step of:
(i) converting 4-hydroxy piperidine (F) to the N-protected-4-hydroxy piperidine (G):

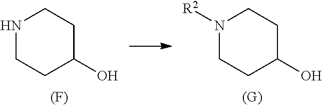

wherein R² is a nitrogen protecting group.

In other words, in one embodiment of the first aspect of the invention, there is provided a process of preparing a thiourea adduct (I) or a salt thereof, the process comprising the steps of:
(i) converting 4-hydroxy piperidine (F) to a N-protected-4-hydroxy piperidine (G):

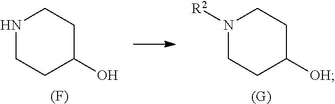

(ii) converting the N-protected-4-hydroxy piperidine (G) to a N-protected-4-derivatised piperidine (H):

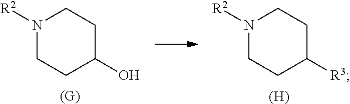

and
(iii) converting the N-protected-4-derivatised piperidine (H) to the thiourea adduct (I) or the salt thereof:

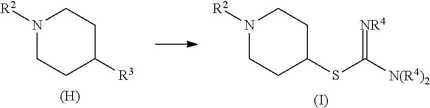

wherein:
R² is a nitrogen protecting group;
R³ is a leaving group; and
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

As will be appreciated, where the first aspect of the invention comprises steps (i), (ii) and (iii), R² is the same in each step, and R³ is the same in steps (ii) and (iii). All optional, typical and exemplary embodiments as described above in relation to the first aspect of the invention apply equally to steps (ii) and (iii) of the present embodiment.

In one embodiment of the first aspect of the invention, the reaction step (i) comprises contacting the 4-hydroxy piperidine (F) with a nitrogen protecting group precursor. In one embodiment, the nitrogen protecting group precursor is X²—R², wherein X² is a leaving group and R² is as defined above. For example, X²—R² may be X²—CH₂R²⁰, wherein R²⁰ is as defined above and X² is selected from Cl, Br, I, or a sulfonate leaving group such as a toluenesulfonate, methanesulfonate, or trifluoromethanesulfonate leaving group. Typically in such an embodiment, X² is selected from Cl or Br. In one aspect of such an embodiment, X²—R² is Br—CH₂R²⁰, such as Br—CH₂Ph. Alternately, X²—R² may be X²—COOCH₂R²⁰, wherein R²⁰ is as defined above and X² is selected from Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the phosphorous or nitrogen atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups RX, wherein each RX is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically where X²—R² is X²—COOCH₂R²⁰, X² is selected from Cl, Br or I. More typically in such an embodiment, X²—R² is Cl—COOCH₂R²⁰, most typically Cl—COOCH₂Ph.

Typically, the reaction step (i) is carried out in the presence of a solvent. Typically, the solvent is a polar solvent or a mixture of polar and non-polar solvents. For example, the solvent may comprise one or more polar protic solvents and/or one or more polar aprotic solvents and/or one or more non-polar solvents. Suitable polar protic solvents include water and alcohols such as methanol, ethanol, n-propanol, isopropanol, n-butanol, sec-butanol, iso-butanol, tert-butanol and tert-amyl alcohol. Suitable polar aprotic solvents include dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate and N-methyl pyrrolidone. Suitable non-polar solvents include pentane, cyclopentane, hexane, cyclohexane, diethyl ether and toluene.

In one embodiment, the reaction step (i) is carried out in the presence of a polar protic solvent such as water, a polar aprotic solvent such as 1,4-dioxane, and a non-polar solvent such as toluene. Typically, in such an embodiment, the solvent mixture comprises from 30 to 50 vol. % of the polar protic solvent, from 30 to 50 vol. % of the polar aprotic solvent, and from 10 to 30 vol. % of the non-polar solvent.

In one embodiment, the reaction step (i) is carried out in the presence of a polar protic solvent such as water, and a non-polar solvent such as toluene. Typically in such an embodiment, the solvent system is biphasic. Typically in such an embodiment, the reaction step (i) is carried out in the absence or the substantial absence of a polar aprotic solvent. Typically, in such an embodiment, the solvent mixture comprises from to 70 vol. % of the polar protic solvent and from 30 to 85 vol. % of the non-polar solvent. More typically in such an embodiment, the solvent mixture comprises from 25 to 45 vol. % of the polar protic solvent and from 55 to 75 vol. % of the non-polar solvent.

As used herein, where it is stated that a reaction or process step is performed in the “substantial absence” of a specified substance or a specified solvent, or that a reaction mixture or solvent system contains “substantially no” specified substance or specified solvent, it is typically to be understood that the reaction mixture or solvent system contains less than 1% by weight of the specified substance or specified solvent. More typically, the reaction mixture or solvent system contains less than 0.1% by weight of the specified substance or specified solvent. More typically still, the reaction mixture or solvent system contains less than 0.01% by weight of the specified substance or specified solvent. Most typically, the reaction mixture or solvent system contains no detectable amount of the specified substance or specified solvent.

Typically, the reaction step (i) comprises contacting the 4-hydroxy piperidine (F) with the nitrogen protecting group precursor (e.g. X²—R² or Cl—COOCH₂Ph) in the presence of a base. In one embodiment, the base is selected from a carbonate, hydrogen carbonate, hydroxide or alkoxide base. Typically the base is a hydroxide or alkoxide base such as an alkali metal hydroxide, an alkali earth metal hydroxide, an alkali metal alkoxide, or an alkali earth metal alkoxide. More typically the base is a hydroxide such as an alkali metal hydroxide or an alkali earth metal hydroxide. More typically still, the base is an alkali metal hydroxide such as lithium hydroxide, potassium hydroxide or sodium hydroxide. Most typically, the base is sodium hydroxide.

In an exemplary embodiment of the first aspect of the invention, there is provided a process of preparing benzyl 4-(carbamimidoylthio)-piperidine-1-carboxylate (I′) or a salt thereof, the process comprising the steps of:
(i) contacting 4-hydroxy piperidine (F) with benzyl chloroformate to obtain N-carboxybenzyl-4-hydroxy piperidine (G′):

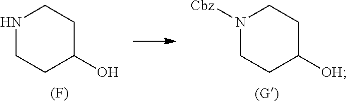

(ii) contacting the N-carboxybenzyl-4-hydroxy piperidine (G′) with mesyl chloride to obtain benzyl 4-((methylsulfonyl)oxy)piperidine-1-carboxylate (H′):

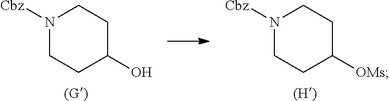

and
(iii) contacting the benzyl 4-((methylsulfonyl)oxy)piperidine-1-carboxylate (H′) with reagent (I-Xb) in a solvent to obtain the benzyl 4-(carbamimidoylthio)-piperidine-1-carboxylate (I′) or the salt thereof:

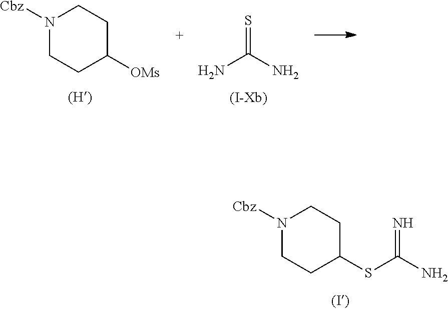

Typically in such an embodiment, in step (i) the 4-hydroxy piperidine (F) is contacted with the benzyl chloroformate in the presence of sodium hydroxide and a solvent.

Typically, where the process of the first aspect of the invention comprises step (i), the 4-hydroxy piperidine (F) is combined with the reaction mixture of step (i) in non-salt form.

Typically, where the process of the first aspect of the invention comprises step (i), the N-protected-4-hydroxy piperidine (G) or (G′) is obtained in step (i) in non-salt form.

In one embodiment of the first aspect of the invention, the N-protected-4-hydroxy piperidine (G) or (G′) is not isolated between steps (i) and (ii).

In another embodiment of the first aspect of the invention, at the end of the reaction the process of step (i) further comprises the work-up step of separating a biphasic mixture comprising the reaction mixture into aqueous and organic phases, wherein the organic phase comprises the N-protected-4-hydroxy piperidine (G) or (G′). In one embodiment the organic phase is washed, e.g. with water. Optionally, the organic phase is dried, e.g. by azeotropic distillation. In one embodiment, the N-protected-4-hydroxy piperidine (G) or (G′) in the organic phase is used directly in step (ii). As will be appreciated, in such an embodiment the solvent of the organic phase provides all or part of the solvent used in the reaction of step (ii).

A second aspect of the invention provides a process of preparing a N-protected-4-(halosulfonyl)-piperidine (J) or a salt thereof, the process comprising the step of converting a thiourea adduct (I) to the N-protected-4-(halosulfonyl)-piperidine (J) or the salt thereof:

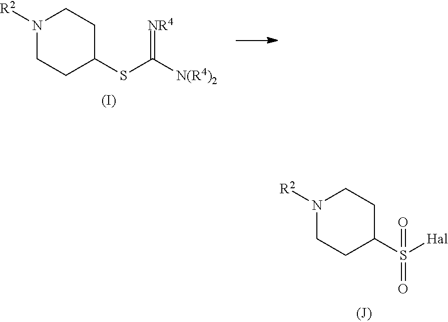

wherein:
R² is a nitrogen protecting group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

As will be appreciated, the embodiments further defining R² and R⁴ in relation to the first aspect of the invention are equally applicable to the second aspect of the invention.

As stated, Hal is Cl or Br. Typically, Hal is Cl.

In one embodiment of the second aspect of the invention, the process comprises the step of contacting the thiourea adduct (I) with a halogenating agent to form the N-protected-4-(halosulfonyl)-piperidine (J) or the salt thereof.

In one embodiment the halogenating agent is selected from N-chlorosuccinimide, 1,3-dichloro-5,5-dimethylhydantoin, trichloroisocyanuric acid, Cl₂, N-bromosuccinimide, 1,3-dibromo-5,5-dimethylhydantoin, tribromoisocyanuric acid and Br₂. Typically, the halogenating agent is selected from N-chlorosuccinimide, 1,3-dichloro-5,5-dimethylhydantoin, trichloroisocyanuric acid, N-bromosuccinimide, 1,3-dibromo-5,5-dimethylhydantoin and tribromoisocyanuric acid. More typically, the halogenating agent is selected from N-chlorosuccinimide and N-bromosuccinimide. Typically, the halogenating agent is a chlorinating agent. Most typically the halogenating agent is N-chlorosuccinimide.

In one embodiment of the second aspect of the invention, the thiourea adduct (I) is contacted with the halogenating agent in the presence of one or more acids and an aqueous solvent.

In one embodiment, the one or more acids are selected from HCl, HBr and carboxylic acids such as formic acid, acetic acid, propionic acid, butyric acid, oxalic acid, malonic acid, succinic acid, tartaric acid, maleic acid and fumaric acid. Typically, at least one acid is a carboxylic acid, more typically a monocarboxylic acid such as formic acid, acetic acid, propionic acid or butyric acid. Most typically, at least one acid is acetic acid. Typically, at least one acid is HCl or HBr. Most typically, at least one acid is HCl.

In one embodiment of the second aspect of the invention, the aqueous solvent is water or a mixture of water and one or more water miscible solvents such as acetonitrile, methanol, ethanol, propanol, acetone, N,N-dimethylformamide, dioxane, or tetrahydrofuran. Typically, the aqueous solvent is water.

In one embodiment, the thiourea adduct (I) is contacted with the halogenating agent in the presence of a carboxylic acid such as formic acid, acetic acid, propionic acid or butyric acid, water, and optionally a second acid selected from HCl or HBr. Typically in such an embodiment, the halogenating agent is selected from N-chlorosuccinimide, 1,3-dichloro-5,5-dimethylhydantoin, trichloroisocyanuric acid, N-bromosuccinimide, 1,3-dibromo-5,5-dimethylhydantoin and tribromoisocyanuric acid. More typically in such an embodiment, the halogenating agent is selected from N-chlorosuccinimide and N-bromosuccinimide. Most typically, the thiourea adduct (I) is contacted with N-chlorosuccinimide in the presence of acetic acid, water, and optionally HCl.

In an exemplary embodiment of the second aspect of the invention, the process comprises the step of contacting benzyl 4-(carbamimidoylthio)-piperidine-1-carboxylate (I′) with a chlorinating agent to obtain benzyl 4-(chlorosulfonyl)-1-piperidinecarboxylate (J′) or a salt thereof:

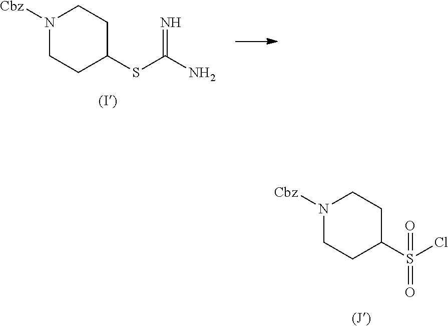 [SMILES: N=C(N)SC1CCN(C(=O)OCc2ccccc2)CC1 ; O=C(OCc1ccccc1)N1CCC(S(=O)(=O)Cl)CC1]

Typically in such an embodiment, the chlorinating agent is N-chlorosuccinimide. Typically in such an embodiment, the benzyl 4-(carbamimidoylthio)-piperidine-1-carboxylate (I′) is contacted with the chlorinating agent in the presence of acetic acid, water and optionally HCl.

Typically in accordance with the second aspect of the invention, the thiourea adduct (I) or (I′) is combined with the reaction mixture in a salt form. More typically, a sulfonic so acid addition salt of the thiourea adduct (I) or (I′) is combined with the reaction mixture. Most typically, a methanesulfonic acid salt of the thiourea adduct (I) or (I′) is combined with the reaction mixture.

Typically in accordance with the second aspect of the invention, the N-protected-4-(halosulfonyl)-piperidine (J) or (J′) is obtained in non-salt form.

In one embodiment of the second aspect of the invention, the step of converting the thiourea adduct (I) or (I′) to the N-protected-4-(halosulfonyl)-piperidine (J) or (J′), or the salt thereof, is carried out at a temperature in the range from 0 to 50° C. Typically, the reaction is carried out at a temperature in the range from 10 to 40° C. More typically, the reaction is carried out at a temperature in the range from 20 to 35° C.

Typically in accordance with the second aspect of the invention, the thiourea adduct (I) or (I′) is present in or added to the solvent at an initial concentration of from 0.1 to 2 mol/L relative to the combined total volume of acid and solvent used in the reaction mixture. More typically, the thiourea adduct (I) or (I′) is present in or added to the solvent at an initial concentration of from 0.3 to 1.5 mol/L. Most typically the thiourea adduct (I) or (I′) is present in or added to the solvent at an initial concentration of from 0.7 to 1.0 mol/L.

Typically in accordance with the second aspect of the invention, the process uses from 1.0 to 5.0 molar equivalents of the halogenating agent, relative to the initial amount of the thiourea adduct (I) or (I′). More typically, the process uses from 2.0 to 4.0 molar equivalents of the halogenating agent. Most typically, the process uses from 2.5 to 3.0 molar equivalents of the halogenating agent.

Typically, where the process of the second aspect of the invention employs one or more acids and an aqueous solvent, the one or more acids comprise from 50 to 99% of the combined total volume of the acids and the solvent. More typically, the one or more acids comprise from 60 to 90% of the combined total volume of the acid and the solvent. More typically still, the one or more acids comprise from 65 to 75% of the combined total volume of the acid and the solvent.

Typically, where the process of the second aspect of the invention employs one or more acids and an aqueous solvent, the water comprises from 1 to 50% of the combined total volume of the acids and the solvent. More typically, the water comprises from 10 to 40% of the combined total volume of the acids and the solvent. More typically still, the water comprises from 25 to 35% of the combined total volume of the acids and the solvent.

Where the process of the second aspect of the invention employs a carboxylic acid and a second acid selected from HCl or HBr, typically the molar ratio of the carboxylic acid to the second acid is from 2:1 to 50:1. More typically, the molar ratio is from 5:1 to 20:1. More typically still, the molar ratio is from 10:1 to 15:1.

In one embodiment of the second aspect of the invention, the process comprises the steps of:
(1) combining the thiourea adduct (I) or (I′) with the one or more acids and the aqueous solvent to form a first mixture; and
(2) adding the halogenating agent to the mixture formed in step (1) to form a second mixture.

Typically, the halogenating agent is added portionwise or continuously to the mixture formed in step (1) over a period of at least 30 minutes. More typically, the halogenating agent is added portionwise or continuously to the mixture formed in step (1) over a period of at least 60 minutes.

In one embodiment of the second aspect of the invention, the process further comprises the work-up steps of:
(3) quenching any residual halogenating agent, for example with a sulfite such as Na₂SO₃;
(4) optionally adding additional water;
(5) allowing a precipitate to form;
(6) isolating the precipitate, e.g. by filtration, to afford the N-protected-4-(halosulfonyl)-piperidine (J) or (J′), or the salt thereof;
(7) optionally washing the precipitate, e.g. with water and/or a mixture of water and acetic acid; and
(8) optionally drying the precipitate, e.g. under vacuum.

Typically in accordance with the second aspect of the invention, steps (4), (7) and (8) are not optional.

Optionally, the reaction mixture is seeded with crystals of the N-protected-4-(halosulfonyl)-piperidine (J) or (J′), or the salt thereof. The seeding may occur during and/or after the addition of the halogenating agent in step (2).

In one embodiment of the second aspect of the present invention, the thiourea adduct (I) or the salt thereof is prepared by a process of the first aspect of the present invention. In other words, in such an embodiment the invention provides a process of preparing a N-protected-4-(halosulfonyl)-piperidine (J) or a salt thereof, the process comprising the steps of:
(iii) converting a N-protected-4-derivatised piperidine (H) to a thiourea adduct (I):

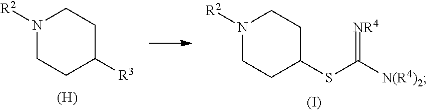

and
(iv) converting the thiourea adduct (I) to the N-protected-4-(halosulfonyl)-piperidine (J) or the salt thereof:

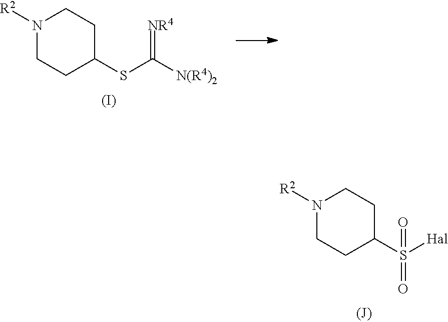

wherein:
R² is a nitrogen protecting group;
R³ is a leaving group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

Typically in such an embodiment, the N-protected-4-derivatised piperidine (H) is prepared by a process of step (ii), or by a process of steps (i) and (ii), as defined above in relation to the first aspect of the invention.

As will be appreciated, where the second aspect of the invention comprises steps (iii) and (iv), R² is the same in each step, and each R⁴ is the same in each step.

All optional, typical and exemplary embodiments as described herein in relation to the first aspect of the invention apply equally to step (iii), and all optional, typical and exemplary embodiments as described herein in relation to the second aspect of the invention apply equally to step (iv). Accordingly, in an exemplary embodiment of the second aspect of the invention, there is provided a process of preparing benzyl 4-(chlorosulfonyl)-1-piperidinecarboxylate (J′) or a salt thereof, the process comprising the steps of:
(iii) contacting benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′):

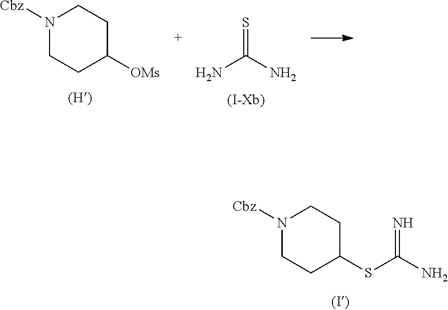

and
(iv) contacting the benzyl 4-(carbamimidoylthio)-piperidine-1-carboxylate (I′) with a chlorinating agent to obtain the benzyl 4-(chlorosulfonyl)-1-piperidinecarboxylate (J′) or the salt thereof:

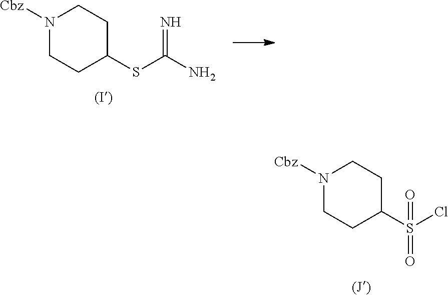 [SMILES: N=C(N)SC1CCN(C(=O)OCc2ccccc2)CC1 ; O=C(OCc1ccccc1)N1CCC(S(=O)(=O)Cl)CC1]

Typically, where the process of the second aspect of the invention comprises step (iii), the N-protected-4-derivatised piperidine (H) or (H′) is combined with the reaction mixture of step (iii) in non-salt form.

Typically, where the process of the second aspect of the invention comprises step (iii), the reagent (I-X), (I-Xa) or (I-Xb) is combined with the reaction mixture of step (iii) in non-salt form.

Typically, where the process of the second aspect of the invention comprises step (iii), the thiourea adduct (I) or (I′) is obtained in step (iii) in salt form. More typically, the thiourea adduct (I) or (I′) is obtained as a sulfonic acid addition salt. Most typically, the thiourea adduct (I) or (I′) is obtained as a methanesulfonic acid salt.

In one embodiment of the second aspect of the invention, the process further comprises the step of:
(v) converting the N-protected-4-(halosulfonyl)-piperidine (J) to a N-protected-4-piperidinesulfonamide (K) or a salt thereof:

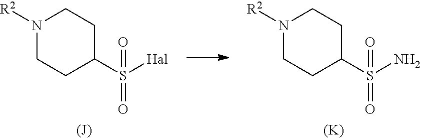

wherein R² is a nitrogen protecting group and Hal is Cl or Br.

In other words, in one embodiment of the second aspect of the invention, there is provided a process of preparing a N-protected-4-piperidinesulfonamide (K) or a salt thereof, the process comprising at least the steps of:
(iv) converting a thiourea adduct (I) to a N-protected-4-(halosulfonyl)-piperidine (J):

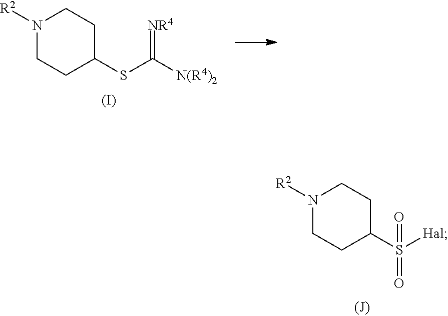

and
(v) converting the N-protected-4-(halosulfonyl)-piperidine (J) to the N-protected-4-piperidinesulfonamide (K) or the salt thereof:

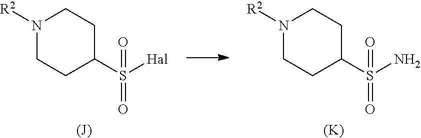

wherein:
R² is a nitrogen protecting group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

As will be appreciated, where the second aspect of the invention comprises steps (iv) and (v), R² is the same in each step and Hal is the same in each step.

In one embodiment of the second aspect of the invention, the reaction step (v) comprises contacting the N-protected-4-(halosulfonyl)-piperidine (J) with ammonia to form the N-protected-4-piperidinesulfonamide (K) or the salt thereof. Typically, the N-protected-4-(halosulfonyl)-piperidine (J) is contacted with ammonia in the presence of a solvent.

Typically, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. For example, the solvent may be selected from dimethyl sulfoxide, tetrahydrofuran, 1,4-dioxane, acetonitrile, dichloromethane, hexamethylphosphoramide, nitromethane, or a mixture thereof. More typically still, the solvent does not comprise a carbonyl, C═N or C≡N group. For example, the solvent may be selected from dimethyl sulfoxide, tetrahydrofuran, 1,4-dioxane, dichloromethane, hexamethylphosphoramide, nitromethane, or a mixture thereof. Yet more typically, the solvent is non-halogenated. For example, the solvent may be selected from dimethyl sulfoxide, tetrahydrofuran, 1,4-dioxane, hexamethylphosphoramide, nitromethane, or a mixture thereof. Most typically, the solvent is tetrahydrofuran.

In one embodiment, the reaction step (v) comprises purging a solution of the N-protected-4-(halosulfonyl)-piperidine (J) in the solvent with ammonia gas.

In another embodiment, the reaction step (v) comprises the steps of:
(1) forming a solution of ammonia in a solvent; and
(2) adding the N-protected-4-(halosulfonyl)-piperidine (J) to the solution formed in step (1).

Typically, in step (1) of step (v), a 100% saturated solution of ammonia in the solvent is formed. More typically, a 25% or 50% saturated solution of ammonia in the solvent is formed. More typically still, a 75% saturated solution of ammonia in the solvent is formed. Most typically, a saturated solution of ammonia in the solvent is formed.

Typically, in step (2) the N-protected-4-(halosulfonyl)-piperidine (J) or (J′) is added portionwise or continuously to the solution formed in step (1) over a period of at least 30 minutes. More typically, the N-protected-4-(halosulfonyl)-piperidine (J) or (J′) is added portionwise or continuously to the solution formed in step (1) over a period of at least 60 minutes.

Optionally in step (2), a solution of the N-protected-4-(halosulfonyl)-piperidine (J) in a second solvent is added to the solution formed in step (1). Typically, the second solvent is the same as the (first) solvent used in step (1).

Typically in any embodiment of step (v) of the second aspect of the invention, the N-protected-4-(halosulfonyl)-piperidine (J) is contacted with ammonia or the ammonia solution in the absence or substantial absence of water and alcohols. More typically, the N-protected-4-(halosulfonyl)-piperidine (J) is contacted with ammonia or the ammonia solution in the absence or substantial absence of polar protic solvents.

In an exemplary embodiment of the second aspect of the invention, the reaction step (v) comprises contacting benzyl 4-(chlorosulfonyl)-1-piperidinecarboxylate (J′) with ammonia to obtain 1-(benzyloxycarbonyl)-4-piperidinesulfonamide (K′) or a salt thereof:

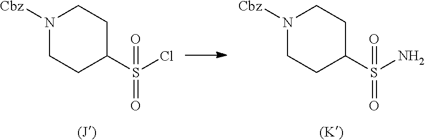 [SMILES: O=C(OCc1ccccc1)N1CCC(S(=O)(=O)Cl)CC1 ; NS(=O)(=O)C1CCN(C(=O)OCc2ccccc2)CC1]

Typically in such an embodiment, the reaction step (v) comprises forming a solution of ammonia in a solvent, and adding the benzyl 4-(chlorosulfonyl)-1-piperidinecarboxylate (J′) to the formed solution to the obtain 1-(benzyloxycarbonyl)-4-piperidinesulfonamide (K′) or a salt thereof. Typically, the solvent is a polar aprotic solvent such as tetrahydrofuran. Typically, the solution of ammonia is a saturated solution of ammonia in the solvent.

Typically, where the process of the second aspect of the invention comprises step (v), the N-protected-4-(halosulfonyl)-piperidine (J) or (J′) is combined with the reaction mixture of step (v) in non-salt form.

Typically, where the process of the second aspect of the invention comprises step (v), so the N-protected-4-piperidinesulfonamide (K) or (K′) is obtained in step (v) in non-salt form.

In one embodiment of the second aspect of the invention, the N-protected-4-piperidinesulfonamide (K) or (K′) is isolated by crystallisation.

In one embodiment of the second aspect of the invention, the process further comprises the step of:
(vi) converting the N-protected-4-piperidinesulfonamide (K) to 1-ethyl-4-piperidinesulfonamide (A) or a salt thereof:

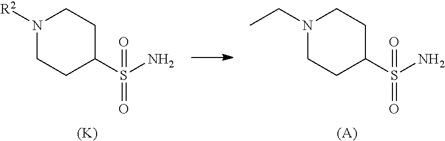

wherein R² is a nitrogen protecting group.

In other words, in one embodiment of the second aspect of the invention, there is provided a process of preparing 1-ethyl-4-piperidinesulfonamide (A) or a salt thereof, the process comprising at least the steps of:
(iv) converting a thiourea adduct (I) to a N-protected-4-(halosulfonyl)-piperidine (J):

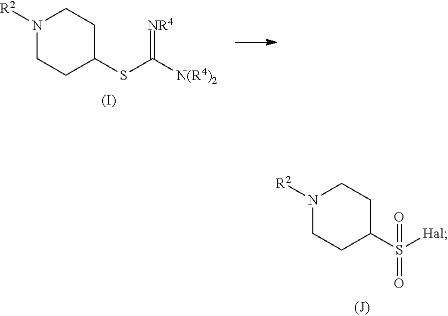

(v) converting the N-protected-4-(halosulfonyl)-piperidine (J) to a N-protected-4-piperidinesulfonamide (K):

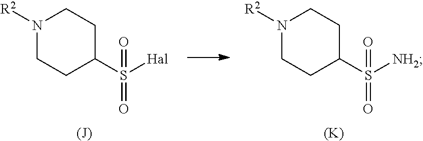

and
(vi) converting the N-protected-4-piperidinesulfonamide (K) to 1-ethyl-4-piperidinesulfonamide (A) or the salt thereof:

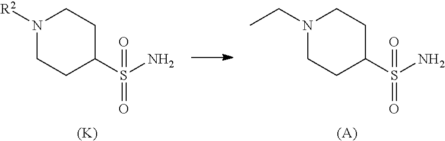

wherein:
R² is a nitrogen protecting group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

As will be appreciated, where the second aspect of the invention comprises steps (iv), (v) and (vi), R² is the same in each step.

In one embodiment of the second aspect of the invention, the reaction step (vi) comprises the steps of:
(vi-a) de-protecting the N-protected-4-piperidinesulfonamide (K) to form piperidine-4-sulfonamide; and
(vi-b) alkylating the piperidine-4-sulfonamide to form the 1-ethyl-4-piperidine-sulfonamide (A) or the salt thereof.

As will be understood, the reaction conditions for the de-protection step (vi-a) will correspond to the nitrogen protecting group being removed. For example, where R² is a benzyloxycarbonyl (CBz), 4-methoxy-benzyloxycarbonyl, benzyl, —CH₂R²⁰ or —COOCH₂R²⁰ group it may be removed by catalytic hydrogenolysis or by treatment with HBr in a carboxylic acid such as acetic or trifluoroacetic acid. Where R² is a t-butoxycarbonyl (Boc) group, it may be removed under acidic conditions, e.g. by treatment with trifluoroacetic acid. Where R² is a 2-(4-biphenylyl)-isopropoxycarbonyl (Bpoc) or triphenylmethyl (Trt) group, it may be removed under acidic conditions, e.g. by treatment with trifluoroacetic acid, or by catalytic hydrogenolysis. Where R² is a 2,2,2-trichloroethoxycarbonyl (Troc) group, it may be removed by treatment with zinc in acetic acid. Conditions suitable for deprotection may be found by reference to e.g. Wuts, ‘Greene's Protective Groups in Organic Synthesis’, 5^{th} Ed., 2014, the contents of which are incorporated herein by reference in their entirety.

Typically in accordance with the first and second aspect of the invention, R² is a nitrogen protecting group that may be removed by catalytic hydrogenolysis. Where the nitrogen protecting group is removed by catalytic hydrogenolysis, typically the process of step (vi-a) comprises contacting the N-protected-4-piperidinesulfonamide (K) with a catalyst in the presence of hydrogen gas. Suitable catalysts include Raney nickel and palladium catalysts. In one embodiment, the catalyst is a palladium catalyst, for example palladium on carbon or palladium hydroxide on carbon. Typically, the catalyst is palladium on carbon on carbon. Typically, the hydrogen gas is used at a pressure in the range from 0.1 to 100 Bar. In one embodiment, the hydrogen gas is used at a pressure in the range from 0.5 to 50 Bar, and more typically in the range from 5 to 25 Bar.

Typically, the N-protected-4-piperidinesulfonamide (K) is contacted with the catalyst in the presence of hydrogen gas and a solvent. Typically, the solvent is a polar protic solvent, or a polar aprotic solvent, or a mixture thereof. For example, the solvent may so be selected from tetrahydrofuran, 1,4-dioxane, acetonitrile, dichloromethane, water, alcohols such as methanol, ethanol, isopropanol and butanol, or a mixture thereof.

Typically, the catalytic hydrogenolysis of step (vi-a) is carried out at a temperature in the range from 0 to 100° C. In one embodiment of the second aspect of the invention, the catalytic hydrogenolysis of step (vi-a) is carried out at a temperature in the range from 15 to 80° C. Typically in such an embodiment, the catalytic hydrogenolysis of step (vi-a) is carried out at a temperature in the range from 20 to 70° C. More typically, the catalytic hydrogenolysis of step (vi-a) is carried out at a temperature in the range from 55 to 65° C.

The alkylation step (vi-b) may be performed under a variety of conditions.

In one embodiment, the alkylation step (vi-b) comprises contacting the piperidine-4-sulfonamide with Et-X^{f}, wherein X^{f} is a leaving group. Typically in such an embodiment, X^{f} is selected from Cl, Br, I, or a sulfonate leaving group such as a toluenesulfonate, methanesulfonate, or trifluoromethanesulfonate leaving group. More typically, X^{f} is selected from Cl, Br or I.

In one embodiment, the piperidine-4-sulfonamide is contacted with Et-X^{f} in the presence of a solvent and optionally a base. Typically the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the base is a carbonate base, such as an alkali metal or alkali earth metal carbonate.

In another embodiment, the piperidine-4-sulfonamide is alkylated by reductive alkylation. For example, the piperidine-4-sulfonamide may be contacted with acetonitrile or acetaldehyde in the presence of a hydride source such as NaCNBH₃.

Alternatively, the piperidine-4-sulfonamide may be contacted with acetonitrile or acetaldehyde in the presence of a catalyst and hydrogen gas. Typically, the piperidine-4-sulfonamide is contacted with acetonitrile in the presence of a catalyst and hydrogen gas. Suitable catalysts include Raney nickel and palladium catalysts. In one embodiment, the catalyst is a palladium catalyst, for example palladium on carbon or so palladium hydroxide on carbon. Typically, the catalyst is palladium on carbon. Typically, the hydrogen gas is used at a pressure in the range from 0.1 to 100 Bar. In one embodiment, the hydrogen gas is used at a pressure in the range from 0.5 to 50 Bar, and more typically in the range from 5 to 25 Bar.

Where the piperidine-4-sulfonamide is contacted with acetonitrile or acetaldehyde, in one embodiment the acetonitrile or acetaldehyde, or a mixture of the acetonitrile or acetaldehyde with water, is used as the solvent.

In another embodiment, where the piperidine-4-sulfonamide is contacted with acetonitrile or acetaldehyde, the contact takes place in the presence of a solvent. Typically the solvent is a polar protic solvent, or a polar aprotic solvent (other than acetonitrile or acetaldehyde), or a mixture thereof. For example, the solvent may be selected from tetrahydrofuran, 1,4-dioxane, dichloromethane, water, an alcohol such as methanol, ethanol, isopropanol or butanol, or a mixture thereof. More typically, the solvent is a polar protic solvent such as water or an alcohol, or a mixture thereof.

In one embodiment, where the piperidine-4-sulfonamide is contacted with acetonitrile, the contact takes place in the presence of water and an alcohol. Typically, the alcohol is selected from the group consisting of methanol, ethanol, n-propanol, isopropanol, n-butanol, sec-butanol, iso-butanol, tert-butanol, tert-amyl alcohol, and mixtures thereof. More typically, the contact takes place in the presence of water and butanol, most typically water and n-butanol.

Typically, the alkylation of step (vi-b) is carried out at a temperature in the range from 0 to 100° C. In one embodiment of the second aspect of the invention, the alkylation of step (vi-b) is carried out at a temperature in the range from 15 to 80° C. Typically in such an embodiment, the alkylation of step (vi-b) is carried out at a temperature in the range from 20 to 70° C. More typically, the alkylation of step (vi-b) is carried out at a temperature in the range from 55 to 65° C.

As will be appreciated, advantageously where R² is a nitrogen protecting group that may be removed by catalytic hydrogenolysis, the steps of:
(vi-a) de-protecting the N-protected-4-piperidinesulfonamide (K) to form piperidine-4-sulfonamide; and
(vi-b) alkylating the piperidine-4-sulfonamide to form the 1-ethyl-4-piperidine-sulfonamide (A) or the salt thereof,
may be performed simultaneously or sequentially in a one-pot reaction.

Accordingly, in one embodiment of the second aspect of the invention, where R² is a nitrogen protecting group that may be removed by catalytic hydrogenolysis, the reaction step (vi) comprises contacting the N-protected-4-piperidinesulfonamide (K) with acetonitrile or acetaldehyde in the presence of a catalyst and hydrogen gas, to obtain 1-ethyl-4-piperadinesulfonamide (A) or a salt thereof. Typically in such an embodiment, the reaction step (f) comprises contacting the N-protected-4-piperidinesulfonamide (K) with acetonitrile in the presence of a catalyst and hydrogen gas. Suitable catalysts include Raney nickel and palladium catalysts. In one embodiment, the catalyst is a palladium catalyst, for example palladium on carbon or palladium hydroxide on carbon. Typically, the catalyst is palladium on carbon.

In an exemplary embodiment of the second aspect of the invention, the reaction step (vi) comprises contacting 1-(benzyloxycarbonyl)-4-piperidinesulfonamide (K′) with acetonitrile or acetaldehyde in the presence of a catalyst and hydrogen gas, to obtain 1-ethyl-4-piperadinesulfonamide (A) or a salt thereof:

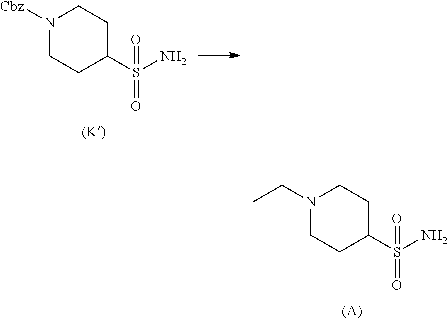 [SMILES: NS(=O)(=O)C1CCN(C(=O)OCc2ccccc2)CC1 ; CCN1CCC(S(N)(=O)=O)CC1 ; "(A)"]

Typically in such an embodiment, the 1-(benzyloxycarbonyl)-4-piperidinesulfonamide (K′) is contacted with acetonitrile in the presence of a catalyst and hydrogen gas. Typically the catalyst is a palladium catalyst such as palladium on carbon.

Typically, where the process of the second aspect of the invention comprises step (vi), the N-protected-4-piperidinesulfonamide (K) or (K′) is combined with the reaction mixture of step (vi) in non-salt form.

Typically, where the process of the second aspect of the invention comprises step (vi), the 1-ethyl-4-piperadinesulfonamide (A) is obtained in step (vi) in non-salt form.

Where reaction step (vi) comprises contacting the N-protected-4-piperidinesulfonamide (K) or (K′) with acetonitrile or acetaldehyde in the presence of a catalyst and hydrogen gas, typically the hydrogen gas is used at a pressure in the range from 0.1 to 100 Bar. In one embodiment, the hydrogen gas is used at a pressure in the range from 0.5 to 50 Bar, and more typically in the range from 5 to 25 Bar.

Where the N-protected-4-piperidinesulfonamide (K) or (K′) is contacted with acetonitrile or acetaldehyde, in one embodiment the acetonitrile or acetaldehyde, or a mixture of the acetonitrile or acetaldehyde with water, is used as the solvent.

In another embodiment, where the N-protected-4-piperidinesulfonamide (K) or (K′) thereof is contacted with acetonitrile or acetaldehyde, the contact takes place in the presence of a solvent. Typically the solvent is a polar protic solvent, or a polar aprotic solvent (other than acetonitrile or acetaldehyde), or a mixture thereof. For example, the solvent may be selected from tetrahydrofuran, 1,4-dioxane, dichloromethane, water, an alcohol such as methanol, ethanol, isopropanol or butanol, or a mixture thereof. More typically, the solvent is a polar protic solvent such as water or an alcohol, or a mixture thereof.

In one embodiment, where the N-protected-4-piperidinesulfonamide (K) or (K′) is contacted with acetonitrile, the contact takes place in the presence of water and an alcohol. Typically, where the contact takes place in the presence of water and an alcohol, the ratio of water:alcohol present is from 1:1 to 1:10 by volume. More typically, the ratio of water:alcohol is from 1:2 to 1:5 by volume. Typically, the alcohol is selected from the group consisting of methanol, ethanol, n-propanol, isopropanol, n-butanol, sec-butanol, iso-butanol, tert-butanol, tert-amyl alcohol, and mixtures thereof. More typically, the contact takes place in the presence of water and ethanol, or water and butanol (such as n-butanol).

Where reaction step (vi) comprises contacting the N-protected-4-piperidine-sulfonamide (K) or (K′) with acetonitrile or acetaldehyde in the presence of a catalyst and hydrogen gas, typically the reaction step (vi) is carried out at a temperature in the range from 0 to 100° C. In one embodiment, the reaction step (vi) is carried out at a temperature in the range from 15 to 80° C. Typically in such an embodiment, the reaction step (vi) is carried out at a temperature in the range from 20 to 70° C. More typically in such an embodiment, the reaction step (vi) is carried out at a temperature in the range from 55 to 65° C.

Where the catalyst in any of steps (vi), (vi-a) or (vi-b) is palladium on carbon or palladium hydroxide on carbon, typically from 2 to 35 wt. % palladium or palladium hydroxide on carbon is used. More typically, from 5 to 30 wt. % palladium or palladium hydroxide on carbon is used. Most typically, from 5 to 15 wt. % palladium or palladium hydroxide on carbon is used.

As will be appreciated, where the second aspect of the invention includes comprises steps (iii) and (iv) as outlined above, the N-protected-4-derivatised piperidine (H) or (H′) may be prepared by a process of step (ii) or a process of steps (i) and (ii) of the first aspect of the invention.

Accordingly, in one embodiment of the second aspect of the invention, there is provided a process of preparing a N-protected-4-piperidinesulfonamide (K) or a salt thereof, the process comprising at least the steps of:
(i) converting 4-hydroxy piperidine (F) to a N-protected-4-hydroxy piperidine (G):

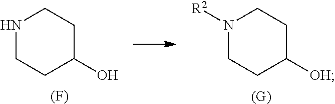

(ii) converting the N-protected-4-hydroxy piperidine (G) to a N-protected-4-derivatised piperidine (H):

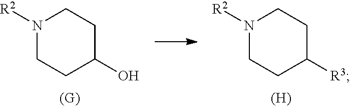

(iii) converting the N-protected-4-derivatised piperidine to a thiourea adduct (I):

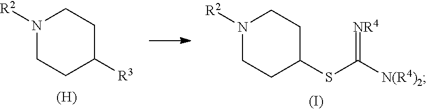

(iv) converting the thiourea adduct (I) to a N-protected-4-(halosulfonyl)-piperidine (J):

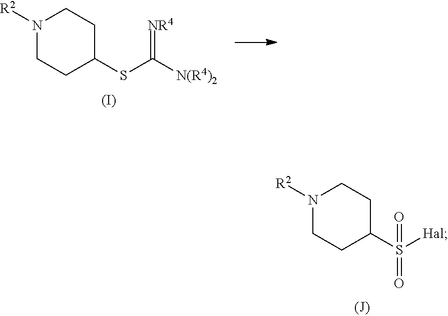

and
(v) converting the N-protected-4-(halosulfonyl)-piperidine (J) to the N-protected-4-piperidinesulfonamide (K) or the salt thereof:

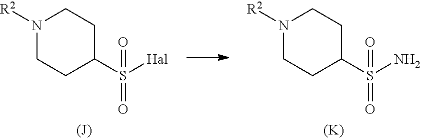

wherein:
R² is a nitrogen protecting group;
R³ is a leaving group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each 20 R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

In an exemplary illustration of such an embodiment, there is provided a process of preparing 1-(benzyloxycarbonyl)-4-piperidinesulfonamide (K′) or a salt thereof, the process comprising at least the steps of:
(i) contacting 4-hydroxy piperidine (F) with benzyl chloroformate to obtain N-carboxybenzyl-4-hydroxy piperidine (G′):

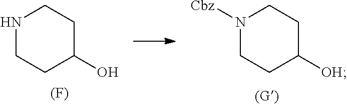

(ii) contacting the N-carboxybenzyl-4-hydroxy piperidine (G′) with mesyl chloride to obtain benzyl 4-((methylsulfonyl)oxy)piperidine-1-carboxylate (H′):

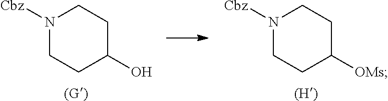

(iii) contacting the benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′):

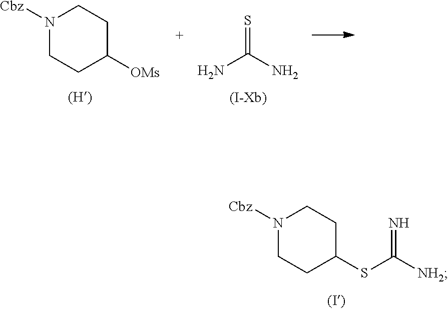

(iv) contacting the benzyl 4-(carbamimidoylthio)-piperidine-1-carboxylate (I′) with a chlorinating agent to obtain benzyl 4-(chlorosulfonyl)-1-piperidinecarboxylate (J′):

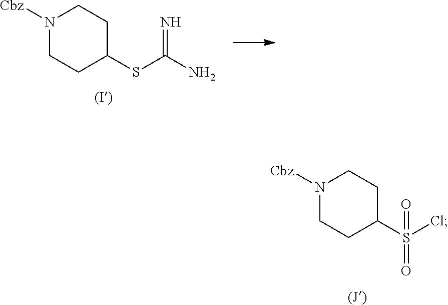 [SMILES: N=C(N)SC1CCN(C(=O)OCc2ccccc2)CC1 ; O=C(OCc1ccccc1)N1CCC(S(=O)(=O)Cl)CC1]

and
(v) contacting the benzyl 4-(chlorosulfonyl)-1-piperidinecarboxylate (J′) with ammonia to obtain the 1-(benzyloxycarbonyl)-4-piperidinesulfonamide (K′) or the salt thereof:

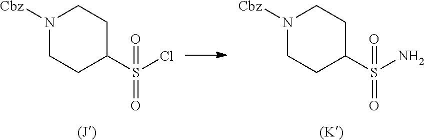 [SMILES: O=C(OCc1ccccc1)N1CCC(S(=O)(=O)Cl)CC1 ; NS(=O)(=O)C1CCN(C(=O)OCc2ccccc2)CC1]

In a further embodiment of the second aspect of the invention, there is provided a process of preparing 1-ethyl-4-piperadinesulfonamide (A) or a salt thereof, the process comprising at least the steps of:
(i) converting 4-hydroxy piperidine (F) to a N-protected-4-hydroxy piperidine (G):

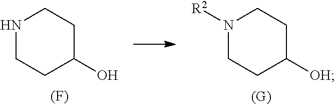

(ii) converting the N-protected-4-hydroxy piperidine (G) to a N-protected-4-derivatised piperidine (H):

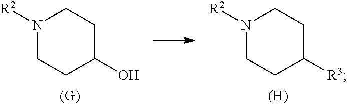

(iii) converting the N-protected-4-derivatised piperidine (H) to a thiourea adduct (I):

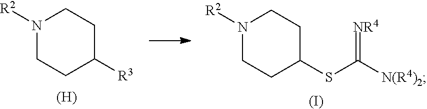

(iv) converting the thiourea adduct (I) to a N-protected-4-(halosulfonyl)-piperidine (J):

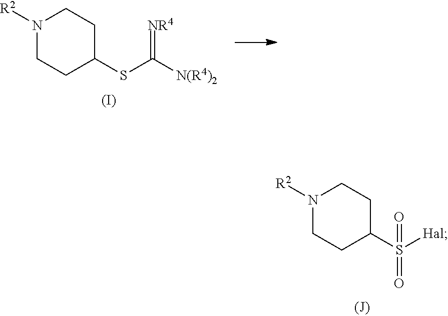

(v) converting the N-protected-4-(halosulfonyl)-piperidine (J) to a N-protected-4-piperidinesulfonamide (K):

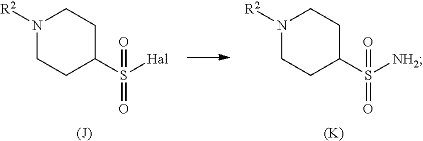

and
(vi) converting the N-protected-4-piperidinesulfonamide (K) to the 1-ethyl-4-piperidinesulfonamide (A) or the salt thereof:

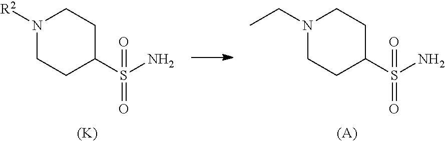

wherein:
R² is a nitrogen protecting group;
R³ is a leaving group;
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

In an exemplary illustration of such an embodiment, there is provided a process of preparing 1-ethyl-4-piperadinesulfonamide (A) or a salt thereof, the process comprising at least the steps of:
(i) contacting 4-hydroxy piperidine (F) with benzyl chloroformate to obtain N-carboxybenzyl-4-hydroxy piperidine (G′):

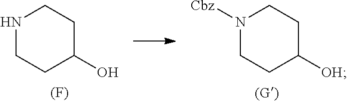

(ii) contacting the N-carboxybenzyl-4-hydroxy piperidine (G′) with mesyl chloride to obtain benzyl 4-((methylsulfonyl)oxy)piperidine-1-carboxylate (H′):

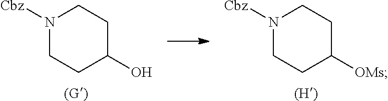

(iii) contacting the benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′):

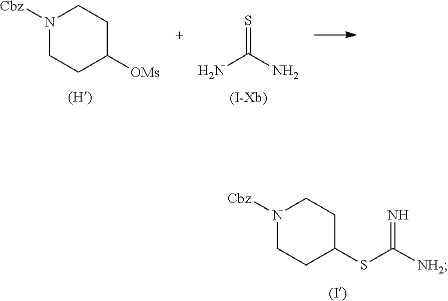

(iv) contacting the benzyl 4-(carbamimidoylthio)-piperidine-1-carboxylate (I′) with a chlorinating agent to obtain benzyl 4-(chlorosulfonyl)-1-piperidinecarboxylate (J′):

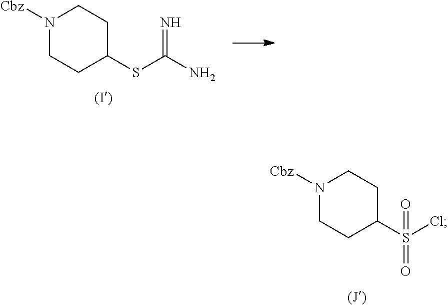 [SMILES: N=C(N)SC1CCN(C(=O)OCc2ccccc2)CC1 ; O=C(OCc1ccccc1)N1CCC(S(=O)(=O)Cl)CC1]

(v) contacting the benzyl 4-(chlorosulfonyl)-1-piperidinecarboxylate (J′) with ammonia to obtain 1-(benzyloxycarbonyl)-4-piperidinesulfonamide (K′):

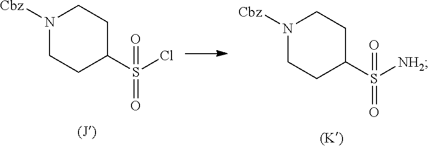 [SMILES: O=C(OCc1ccccc1)N1CCC(S(=O)(=O)Cl)CC1 ; NS(=O)(=O)C1CCN(C(=O)OCc2ccccc2)CC1]

and
(vi) contacting the 1-(benzyloxycarbonyl)-4-piperidinesulfonamide (K′) with acetonitrile or acetaldehyde in the presence of a catalyst and hydrogen gas, to obtain the 1-ethyl-4-piperadinesulfonamide (A) or the salt thereof:

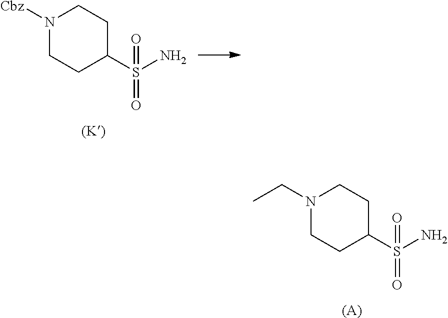 [SMILES: NS(=O)(=O)C1CCN(C(=O)OCc2ccccc2)CC1 ; CCN1CCC(S(N)(=O)=O)CC1 ; "(A)"]

Typically, where the process of the second aspect of the invention comprises step (i), the 4-hydroxy piperidine (F) is combined with the reaction mixture in step (i) in non-salt form.

Typically, where the process of the second aspect of the invention comprises step (i), the N-protected-4-hydroxy piperidine (G) or (G′) is obtained in step (i) in non-salt form.

Typically, where the process of the second aspect of the invention comprises step (ii), the N-protected-4-hydroxy piperidine (G) or (G′) is combined with the reaction mixture in step (ii) in non-salt form.

Typically, where the process of the second aspect of the invention comprises step (ii), the N-protected-4-derivatised piperidine (H) or (H′) is obtained in step (ii) in non-salt form.

A third aspect of the invention provides a process comprising one or more steps selected from:
(i) converting 4-hydroxy piperidine (F) to a N-protected-4-hydroxy piperidine (G):

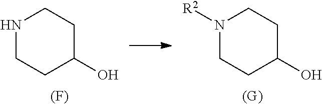

wherein the conversion is performed in a biphasic solvent system;
(ii) converting a N-protected-4-hydroxy piperidine (G) to a N-protected-4-derivatised piperidine (H):

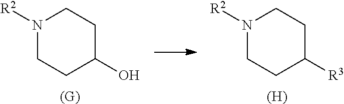

wherein the conversion is performed in the presence of a non-polar solvent;
(iii) converting a N-protected-4-derivatised piperidine (H) to a thiourea adduct (I):

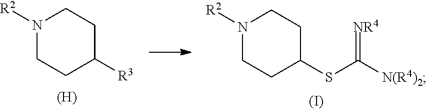

(iv) converting a thiourea adduct (I) to a N-protected-4-(halosulfonyl)-piperidine (J):

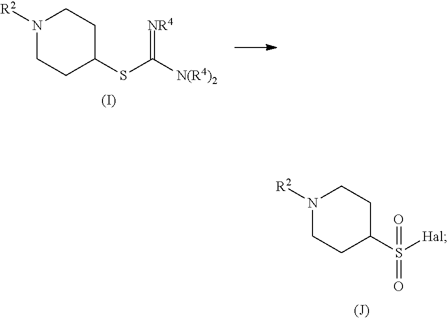

(v) converting a N-protected-4-(halosulfonyl)-piperidine (J) to a N-protected-4-piperidinesulfonamide (K):

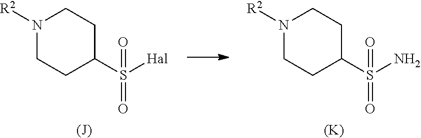

wherein the conversion comprises the steps of (1) forming a solution of ammonia in a solvent, and (2) adding the N-protected-4-(halosulfonyl)-piperidine (J) to the solution formed in step (1); and
(vi) converting a N-protected-4-piperidinesulfonamide (K) to 1-ethyl-4-piperidinesulfonamide (A):

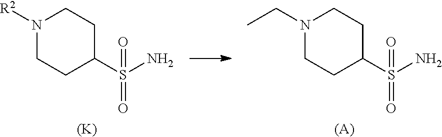

wherein the conversion is performed in the presence of a C₃-C₅ alcohol;
   and wherein:
R² is a nitrogen protecting group;
R³ is a leaving group;
each R⁴ is independently selected from hydrogen or a C₁-C₂₀ hydrocarbyl group, so wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton; and
Hal is Cl or Br.

As will be appreciated, the embodiments further defining R², R³, R⁴ and Hal in relation to the first and second aspect of the invention are equally applicable to the third aspect of the invention.

That said, typically in accordance with any of steps (i)-(vi) of the third aspect of the invention, R² is —CH₂R²⁰ or —COOCH₂R²⁰, wherein R²⁰ is an aryl or heteroaryl group, wherein the aryl or heteroaryl group is monocyclic, bicyclic or tricyclic, wherein the aryl or heteroaryl group may optionally be substituted with one or more substituents independently selected from halo, —CN, —OH, —NO₂, —NH₂, —R²¹, —OR²¹, —NHR²¹, —N(R²¹)₂ or —N(O)(R²¹)₂, wherein each R²¹ is independently selected from a C₁-C₄ alkyl, C₁-C₄ haloalkyl, C₃-C₄ cycloalkyl or C₃-C₄ halocycloalkyl group, or any two R²¹ directly attached to the same nitrogen atom may together form a C₂-C₅ alkylene or C₂-C₅ haloalkylene group, and wherein R²⁰, including any optional substituents, contains from 1 to 20 carbon atoms. More typically, R² is —COOCH₂R²⁰. Most typically, R² is —COOCH₂Ph.

Typically in accordance with step (ii) and/or (iii) of the third aspect of the invention, R³ is a sulfonate leaving group such as a toluenesulfonate (tosylate or —OTs), methanesulfonate (mesylate or —OMs), or trifluoromethanesulfonate (triflate or —OTf) leaving group. Most typically R³ is —OMs.

Typically in accordance with step (iii) and/or (iv) of the third aspect of the invention, each R⁴ is independently selected from hydrogen or a C₁-C₆ alkyl or C₃-C₆ cycloalkyl group, or any two R⁴ may together form a C₂-C₆ alkylene group, wherein any C₁-C₆ alkyl, C₃-C₆ cycloalkyl or C₂-C₆ alkylene group may optionally be fluoro substituted. Most typically, each R⁴ is hydrogen.

Typically in accordance with step (iv) and/or (v) of the third aspect of the invention, Hal is Cl.

It will also be appreciated that steps (i)-(vi) of the third aspect of the invention correspond to the equivalent steps (i)-(vi) of the first and second aspects of the invention. Accordingly, insofar as practicable, any optional, typical or exemplary embodiments as described herein in relation to any of steps (i)-(vi) of the first or second aspects of the invention apply equally to the corresponding steps (i)-(vi) of the third aspect of the invention. Accordingly, in an exemplary embodiment of the third aspect of the invention there is provided a process comprising one or more steps selected from:
(i) contacting 4-hydroxy piperidine (F) with benzyl chloroformate in a biphasic solvent system to obtain N-carboxybenzyl-4-hydroxy piperidine (G′):

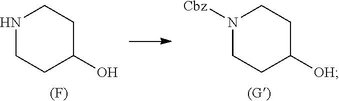

(ii) contacting N-carboxybenzyl-4-hydroxy piperidine (G′) with mesyl chloride in a non-polar solvent to obtain benzyl 4-((methylsulfonyl)oxy)piperidine-1-carboxylate (H′):

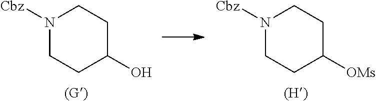

(iii) contacting benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′):

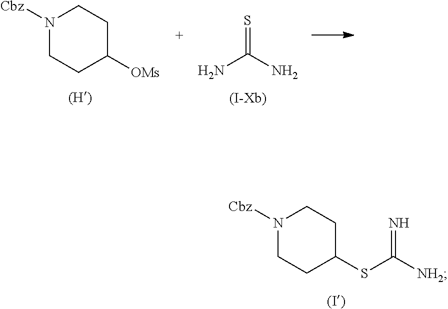

(iv) contacting benzyl 4-(carbamimidoylthio)-piperidine-1-carboxylate (I′) with a chlorinating agent to obtain benzyl 4-(chlorosulfonyl)-1-piperidinecarboxylate (J′):

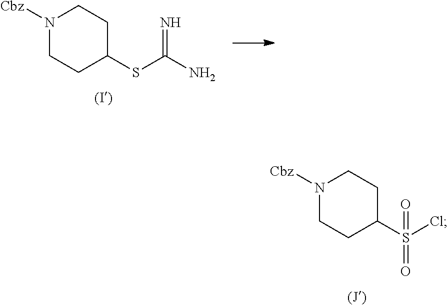 [SMILES: N=C(N)SC1CCN(C(=O)OCc2ccccc2)CC1 ; O=C(OCc1ccccc1)N1CCC(S(=O)(=O)Cl)CC1]

(v) forming a solution of ammonia in a solvent, and adding benzyl 4-(chlorosulfonyl)-1-piperidinecarboxylate (J′) to the formed solution to obtain 1-(benzyloxycarbonyl)-4-piperidinesulfonamide (K′):

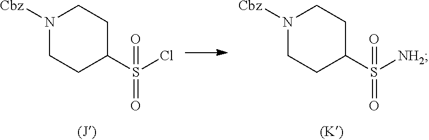 [SMILES: O=C(OCc1ccccc1)N1CCC(S(=O)(=O)Cl)CC1 ; NS(=O)(=O)C1CCN(C(=O)OCc2ccccc2)CC1]

and
(vi) contacting 1-(benzyloxycarbonyl)-4-piperidinesulfonamide (K′) with acetonitrile or acetaldehyde in the presence of a catalyst, hydrogen gas and a C₃-C₅ alcohol, to obtain 1-ethyl-4-piperadinesulfonamide (A):

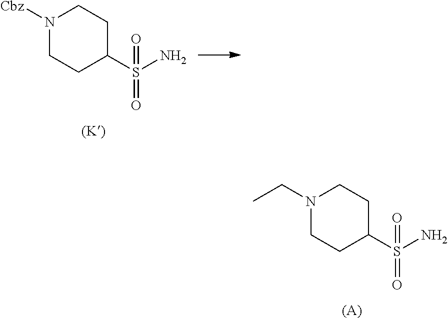 [SMILES: NS(=O)(=O)C1CCN(C(=O)OCc2ccccc2)CC1 ; CCN1CCC(S(N)(=O)=O)CC1 ; "(A)"]

In one embodiment of the third aspect of the invention, the process comprises at least step (i).

Typically, where the process of the third aspect of the invention comprises step (i), the 4-hydroxy piperidine (F) is combined with the reaction mixture in step (i) in non-salt form.

Typically, where the process of the third aspect of the invention comprises step (i), the N-protected-4-hydroxy piperidine (G) or (G′) is obtained in step (i) in non-salt form.

Where the process of the third aspect of the invention comprises step (i), typically the biphasic solvent system of step (i) comprises a polar protic solvent such as water, and a non-polar solvent such as pentane, cyclopentane, hexane, cyclohexane, diethyl ether or toluene. More typically, the biphasic solvent system comprises water and toluene.

Typically, in such an embodiment, the solvent mixture comprises from 15 to 70 vol. % of the polar protic solvent and from 30 to 85 vol. % of the non-polar solvent. More typically in such an embodiment, the solvent mixture comprises from 25 to 45 vol. % of the polar protic solvent and from 55 to 75 vol. % of the non-polar solvent.

Typically, the biphasic solvent system contains no or substantially no 1,4-dioxane. More typically, the biphasic solvent system contains no or substantially no polar aprotic solvent.

In one embodiment of the third aspect of the invention, the reaction step (i) comprises contacting the 4-hydroxy piperidine (F) with a nitrogen protecting group precursor. In one embodiment, the nitrogen protecting group precursor is X²—R², wherein X² is a leaving group and R² is as defined above. For example, X²—R² may be X²—CH₂R²⁰, wherein R²⁰ is as defined above and X² is selected from Cl, Br, I, or a sulfonate leaving group such as a toluenesulfonate, methanesulfonate, or trifluoromethanesulfonate leaving group. Typically in such an embodiment, X² is selected from Cl or Br. In one aspect of such an embodiment, X²—R² is Br—CH₂R²⁰, such as Br—CH₂Ph. Alternately, X²—R² may be X²—COOCH₂R²⁰, wherein R²⁰ is as defined above and X² is selected from Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the phosphorous or nitrogen atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups RX, wherein each RX is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically where X²—R² is X²—COOCH₂R²⁰, X² is selected from Cl, Br or I. More typically in such an embodiment, X²—R² is Cl—COOCH₂R²⁰, most typically Cl—COOCH₂Ph.

Typically, the reaction step (i) comprises contacting the 4-hydroxy piperidine (F) with the nitrogen protecting group precursor (e.g. X²—R² or Cl—COOCH₂Ph) in the presence of a base. In one embodiment, the base is selected from a carbonate, hydrogen carbonate, hydroxide or alkoxide base. Typically the base is a hydroxide or alkoxide base such as an alkali metal hydroxide, an alkali earth metal hydroxide, an alkali metal alkoxide, or an alkali earth metal alkoxide. More typically the base is a hydroxide such as an alkali metal hydroxide or an alkali earth metal hydroxide. More typically still, the base is an alkali metal hydroxide such as lithium hydroxide, potassium hydroxide or sodium hydroxide. Most typically, the base is sodium hydroxide.

In an exemplary embodiment of the third aspect of the invention, the reaction step (i) comprises contacting 4-hydroxy piperidine (F) with benzyl chloroformate in a biphasic solvent system to obtain N-carboxybenzyl-4-hydroxy piperidine (G′) or a salt thereof:

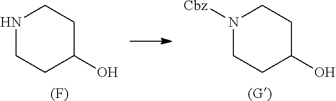

Typically in such an embodiment, the 4-hydroxy piperidine (F) is contacted with the benzyl chloroformate in the presence of sodium hydroxide, and the biphasic solvent system comprises water, a non-polar solvent such as toluene, and substantially no polar aprotic solvent.

In one embodiment of any of the first to third aspects of the invention, the reaction step (i) is carried out at a temperature in the range from −20 to 80° C. Typically, the reaction of step (i) is carried out at a temperature in the range from −10 to 50° C. More typically, the reaction of step (i) is carried out at a temperature in the range from 0 to 30° C.

Typically in accordance with any of the first to third aspects of the invention, in step (i) the 4-hydroxy piperidine (F) is present in or added to the solvent at an initial concentration of from 0.01 to 10 mol/L relative to the total volume of solvent used in the reaction mixture. More typically, the 4-hydroxy piperidine (F) is present in or added to the solvent at an initial concentration of from 0.5 to 2.0 mol/L. Most typically the 4-hydroxy piperidine (F) is present in or added to the solvent at an initial concentration of from 0.8 to 1.5 mol/L.

Typically, where the process of step (i) of any of the first to third aspects of the invention uses X²—COOCH₂R²⁰ (e.g. Cl—COOCH₂Ph) as a nitrogen protecting group precursor, the nitrogen protecting group precursor is contaminated with less than 20 mol % X²—CH₂R²⁰ (e.g. Cl—CH₂Ph). More typically, the nitrogen protecting group precursor is contaminated with less than 10 mol % or less than 5 mol % X²—CH₂R²⁰. Most so typically, the nitrogen protecting group precursor is contaminated with less than 1 mol % X²—CH₂R²⁰. Advantageously, it has been discovered that using a nitrogen protecting group precursor with a low content of such contaminants improves yield and facilitates purification in step (iii).

Typically, the process of step (i) of any of the first to third aspects of the invention uses from 0.5 to 2.0 molar equivalents of the nitrogen protecting group precursor (e.g. X²—R² or Cl—COOCH₂Ph), relative to the initial amount of 4-hydroxy piperidine (F). More typically, the process uses from 0.8 to 1.1 molar equivalents of the nitrogen protecting group precursor. Most typically, the process uses from 0.9 to 1.0 molar equivalents of the nitrogen protecting group precursor.

Typically, the process of step (i) of any of the first to third aspects of the invention uses from 0.8 to 3.0 molar equivalents of the base, relative to the initial amount of 4-hydroxy piperidine (F). More typically, the process uses from 1.0 to 2.0 molar equivalents of the base. Most typically, the process uses from 1.4 to 1.6 molar equivalents of the base.

In one embodiment of any of the first to third aspects of the invention, the process of step (i) comprises the steps of:
(1) combining the 4-hydroxy piperidine (F) and the base with a first portion of the solvent to form a first mixture; and
(2) dissolving the nitrogen protecting group precursor in a second portion of the solvent and adding the resultant solution to the mixture formed in step (1) to form a second mixture.

Typically, the first portion of the solvent comprises or consists of a polar protic solvent such as water and a non-polar solvent such as toluene. Typically, the second portion of the solvent comprises or consists of a non-polar solvent such as toluene.

In one embodiment, step (2) is performed at a temperature in the range from 0 to 10° C. Typically, after step (2) is complete the second mixture is allowed to warm to a temperature in the range from 15 to 30° C.

In one embodiment of the third aspect of the invention, at the end of the reaction the process of step (i) further comprises the work-up step of separating the biphasic reaction mixture into aqueous and organic phases, wherein the organic phase comprises the N-protected-4-hydroxy piperidine (G) or (G′). In one embodiment the organic phase is washed, e.g. with water. Optionally, the organic phase is dried, e.g. by azeotropic distillation. In one embodiment, the solvent is removed from the organic phase to afford the N-protected-4-hydroxy piperidine (G) or (G′). Alternately, the N-protected-4-hydroxy piperidine (G) or (G′) in the organic phase may be used directly in step (ii). As will be appreciated, in such an embodiment the solvent of the organic phase provides all or part of the solvent used in the reaction of step (ii).

In one embodiment of the third aspect of the invention, the process comprises at least step (ii).

Typically, where the process of the third aspect of the invention comprises step (ii), the N-protected-4-hydroxy piperidine (G) or (G′) is combined with the reaction mixture in step (ii) in non-salt form.

Typically, where the process of the third aspect of the invention comprises step (ii), the N-protected-4-derivatised piperidine (H) or (H′) is obtained in step (ii) in non-salt form.

Where the process of the third aspect of the invention comprises step (ii), typically the non-polar solvent of step (ii) is not halogenated. In one embodiment, the non-polar solvent is selected from pentane, cyclopentane, hexane, cyclohexane, diethyl ether, toluene, or a mixture thereof. Most typically, the non-polar solvent is toluene.

In one embodiment of the third aspect of the invention, the reaction step (ii) comprises contacting the N-protected-4-hydroxy piperidine (G) or (G′) with SOCl₂, SOBr₂, or a mixture of Ph₃P and Cl₂ or Br₂, to form the N-protected-4-derivatised piperidine (H), wherein R³ is as appropriate a Cl or Br leaving group.

In another embodiment of the third aspect of the invention, the reaction step (ii) comprises contacting the N-protected-4-hydroxy piperidine (G) or (G′) with a sulfonyl halide or a sulfonyl anhydride to form the N-protected-4-derivatised piperidine (H) or (H′), wherein R³ is a sulfonate leaving group.

As will be appreciated, the sulfonyl halide or sulfonyl anhydride used will correspond to the sulfonate leaving group of R³. For example, where R³ is a tosylate leaving group a tosyl halide or tosyl anhydride will be used. Similarly, where R³ is a mesylate leaving group a mesyl halide or mesyl anhydride will be used, and where R³ is a triflate leaving group a triflic halide or triflic anhydride will be used.

Typically, a sulfonyl halide is used. In one embodiment, the sulfonyl halide is selected from a sulfonyl chloride, a sulfonyl bromide, or a sulfonyl iodide. Typically, the sulfonyl halide is a sulfonyl chloride or a sulfonyl bromide. More typically, the sulfonyl halide is a sulfonyl chloride.

In a typical embodiment of the third aspect of the invention, the reaction step (ii) comprises contacting the N-protected-4-hydroxy piperidine (G) or (G′) with a mesyl halide or mesyl anyhdride to form the N-protected-4-derivatised piperidine (H) or (H′), wherein R³ is a mesylate leaving group. Most typically in such an embodiment the reaction step (ii) comprises contacting the N-protected-4-hydroxy piperidine (G) or (G′) with mesyl chloride.

In one embodiment of the third aspect of the invention, the reaction step (ii) is carried out in the presence of a base. Typically, the reaction step (ii) comprises contacting the N-protected-4-hydroxy piperidine (G) or (G′) with a sulfonyl halide or a sulfonyl anhydride in the presence of a base. Typically, the base is a sterically hindered base. For example, the base may be a tertiary amine such as N,N-diisopropylethylamine (DIPEA), trimethylamine, triethylamine (TEA), tripropylamine or tributylamine. Most typically, the base is triethylamine (TEA).

In an exemplary embodiment of the third aspect of the invention, the reaction step (ii) comprises contacting N-carboxybenzyl-4-hydroxy piperidine (G′) with mesyl chloride in a non-polar solvent to obtain benzyl 4-((methylsulfonyl)oxy)-piperidine-1-carboxylate (H′):

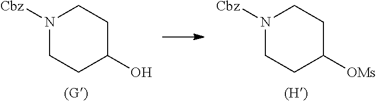

Typically in such an embodiment, the N-carboxybenzyl-4-hydroxy piperidine (G′) is contacted with the mesyl chloride in the presence of a tertiary amine base such as triethylamine. Typically, the non-polar solvent is a non-halogenated non-polar solvent such as toluene.

In one embodiment of any of the first to third aspects of the invention, the reaction step (ii) is carried out at a temperature in the range from −20 to 40° C. Typically, the reaction of step (ii) is carried out at a temperature in the range from −10 to 20° C. More typically, the reaction of step (ii) is carried out at a temperature in the range from −5 to 10° C.

Typically in accordance with any of the first to third aspects of the invention, in step (ii) the N-protected-4-hydroxy piperidine (G) or (G′) is present in or added to the solvent at an initial concentration of from 0.01 to 10 mol/L relative to the total volume of solvent used in the reaction mixture. More typically, the N-protected-4-hydroxy piperidine (G) or (G′) is present in or added to the solvent at an initial concentration of from 0.5 to 2.0 mol/L. Most typically the N-protected-4-hydroxy piperidine (G) or (G′) is present in or added to the solvent at an initial concentration of from 1.2 to 1.6 mol/L.

Typically, the process of step (ii) of any of the first to third aspects of the invention uses from 0.8 to 2.0 molar equivalents of the sulfonyl halide or sulfonyl anyhydride, relative to the initial amount of the N-protected-4-hydroxy piperidine (G) or (G′). More typically, the process uses from 0.9 to 1.5 molar equivalents of the sulfonyl halide or sulfonyl anyhydride. Most typically, the process uses from 1.0 to 1.1 molar equivalents of the sulfonyl halide or sulfonyl anyhydride.

Typically, the process of step (ii) of any of the first to third aspects of the invention uses from 0.9 to 2.0 molar equivalents of the base, relative to the initial amount of the N-protected-4-hydroxy piperidine (G) or (G′). More typically, the process uses from 1.0 to 1.5 molar equivalents of the base. Most typically, the process uses from 1.05 to 1.15 molar equivalents of the base.

In one embodiment of any of the first to third aspects of the invention, the process of step (ii) comprises the steps of:
(1) combining the N-protected-4-hydroxy piperidine (G) or (G′) with the base and the solvent to form a first mixture; and
(2) adding the sulfonyl halide or sulfonyl anhydride to the mixture formed in step (1) to form a second mixture.

Typically in step (2) the sulfonyl halide or sulfonyl anhydride is added dropwise.

In one embodiment of the third aspect of the invention, at the end of the reaction the process of step (ii) further comprises the work-up step of subjecting the reaction mixture to an aqueous wash such that the N-protected-4-derivatised piperidine (H) or (H′) is retained in the organic phase. Typically in such an embodiment, the reaction solvent is toluene. In one embodiment, the solvent is removed from the organic phase to afford the N-protected-4-derivatised piperidine (H) or (H′). Alternately, the N-protected-4-derivatised piperidine (H) or (H′) in the organic phase may be used directly in step (iii). Optionally in such an embodiment, a portion of the solvent, e.g. about 50-75% by volume, is removed from the organic phase, e.g. by distillation under reduced pressure. The remainder of the organic solvent comprising the N-protected-4-derivatised piperidine (H) or (H′) may then be used directly in step (iii). As will be appreciated, in such an embodiment the solvent of the organic phase provides all or part of the solvent used in the reaction of step (iii).

In one embodiment of the third aspect of the invention, the process comprises at least step (iii). As will be appreciated, step (iii) of the third aspect of the invention corresponds to the first aspect of the invention. All optional, typical and exemplary embodiments as described above in relation to the first aspect of the invention apply equally to step (iii) of the third aspect of the invention.

Typically, where the process of the third aspect of the invention comprises step (iii), the N-protected-4-derivatised piperidine (H) or (H′) is combined with the reaction mixture of step (iii) in non-salt form.

Typically, where the process of the third aspect of the invention comprises step (iii), the reagent (I-X), (I-Xa) or (I-Xb) is combined with the reaction mixture of step (iii) in non-salt form.

Typically, where the process of the third aspect of the invention comprises step (iii), the so thiourea adduct (I) or (I′) is obtained in step (iii) in salt form. More typically, the thiourea adduct (I) or (I′) is obtained as a sulfonic acid addition salt. Most typically, the thiourea adduct (I) or (I′) is obtained as a methanesulfonic acid salt.

In one embodiment of the third aspect of the invention, the process comprises at least step (iv). As will be appreciated, step (iv) of the third aspect of the invention corresponds to the second aspect of the invention. All optional, typical and exemplary embodiments as described above in relation to the second aspect of the invention apply equally to step (iv) of the third aspect of the invention.

Typically, where the process of the third aspect of the invention comprises step (iv), the thiourea adduct (I) or (I′) is combined with the reaction mixture of step (iv) in a salt form. More typically, a sulfonic acid addition salt of the thiourea adduct (I) or (I′) is combined with the reaction mixture. Most typically, a methanesulfonic acid salt of the thiourea adduct (I) or (I′) is combined with the reaction mixture.

Typically, where the process of the third aspect of the invention comprises step (iv), the N-protected-4-(halosulfonyl)-piperidine (J) or (J′) is obtained in step (iv) in non-salt form.

In one embodiment of the third aspect of the invention, the process comprises at least step (v).

As stated, the conversion of step (v) of the third aspect of the invention comprises the steps of:
(1) forming a solution of ammonia in a solvent; and
(2) adding the N-protected-4-(halosulfonyl)-piperidine (J) to the solution formed in step (1).

Advantageously, it has been discovered that by adding the N-protected-4-(halosulfonyl)-piperidine (J) or (J′) to a preformed solution of ammonia in a solvent, the N-protected-4-piperidinesulfonamide (K) or (K′) can be obtained in higher purity than if ammonia is passed through a preformed solution of the N-protected-4-(halosulfonyl)-piperidine (J) or (J′) in a solvent.

Typically, where the process of the third aspect of the invention comprises step (v), the N-protected-4-(halosulfonyl)-piperidine (J) or (J′) added to the solution in step (2) of step (v) in non-salt form.

Typically, where the process of the third aspect of the invention comprises step (v), the N-protected-4-piperidinesulfonamide (K) or (K′) is obtained in step (v) in non-salt form.

Typically, in step (1) of step (v), a 10% saturated solution of ammonia in the solvent is formed. More typically, a 25% or 50% saturated solution of ammonia in the solvent is formed. More typically still, a 75% saturated solution of ammonia in the solvent is formed. Most typically, a saturated solution of ammonia in the solvent is formed.

Typically, in step (2) the N-protected-4-(halosulfonyl)-piperidine (J) or (J′) is added portionwise or continuously to the solution formed in step (1) over a period of at least 30 minutes. More typically, the N-protected-4-(halosulfonyl)-piperidine (J) or (J′) is added portionwise or continuously to the solution formed in step (1) over a period of at least 60 minutes.

Typically, the solvent in step (1) of step (v) is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. For example, the solvent may be selected from dimethyl sulfoxide, tetrahydrofuran, 1,4-dioxane, acetonitrile, dichloromethane, hexamethylphosphoramide, nitromethane, or a mixture thereof. More typically still, the solvent does not comprise a carbonyl, C═N or C≡N group. For example, the solvent may be selected from dimethyl sulfoxide, tetrahydrofuran, 1,4-dioxane, dichloromethane, hexamethylphosphoramide, nitromethane, or a mixture thereof. Yet more typically, the solvent is non-halogenated. For example, the solvent may be selected from dimethyl sulfoxide, tetrahydrofuran, 1,4-dioxane, hexamethylphosphoramide, nitromethane, or a mixture thereof. Most typically, the solvent is tetrahydrofuran.

Optionally in step (2), a solution of the N-protected-4-(halosulfonyl)-piperidine (J) in a second solvent is added to the solution formed in step (1). Typically, the second solvent is the same as the (first) solvent used in step (1).

In one embodiment of step (v) of the third aspect of the invention, the step (2) of adding the N-protected-4-(halosulfonyl)-piperidine (J) to the solution formed in step (1) is performed in the absence or substantial absence of water and alcohols. That is to say, the solution of step (1), the N-protected-4-(halosulfonyl)-piperidine (J) used in step (2) and any solution containing the N-protected-4-(halosulfonyl)-piperidine (J) used in step (2) all contain no or substantially no water or alcohols. More typically, the step (2) of adding the N-protected-4-(halosulfonyl)-piperidine (J) to the solution formed in step (1) is performed in the absence or substantial absence of polar protic solvents.

In an exemplary embodiment of the third aspect of the invention, the reaction step (v) comprises forming a solution of ammonia in a solvent, and adding benzyl 4-(chlorosulfonyl)-1-piperidinecarboxylate (J′) to the formed solution to obtain 1-(benzyloxycarbonyl)-4-piperidinesulfonamide (K′):

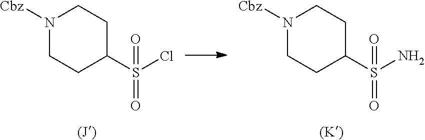 [SMILES: O=C(OCc1ccccc1)N1CCC(S(=O)(=O)Cl)CC1 ; NS(=O)(=O)C1CCN(C(=O)OCc2ccccc2)CC1]

Typically in such an embodiment, the solvent is a non-halogenated polar aprotic solvent such as tetrahydrofuran. Typically, the solution of ammonia is a saturated solution of ammonia in the solvent.

In one embodiment of the second or the third aspect of the invention, in step (v) the N-protected-4-(halosulfonyl)-piperidine (J) or (J′) is combined with ammonia or the solution of ammonia at a temperature in the range from −70 to 30° C. Typically the N-protected-4-(halosulfonyl)-piperidine (J) or (J′) is combined with ammonia or the solution of ammonia at a temperature in the range from −20 to 20° C., more typically in the range from −10 to 10° C.

Typically in accordance with the second aspect or the third aspect of the invention, in step (v) the N-protected-4-(halosulfonyl)-piperidine (J) or (J′) is initially present in or added to the solvent in a total amount of from 0.01 to 10 mol/L, relative to the total volume of solvent used in the reaction mixture. More typically, the N-protected-4-(halosulfonyl)-piperidine (J) or (J′) is initially present in or added to the solvent in a total amount of from 0.1 to 1.0 mol/L. Most typically the N-protected-4-(halosulfonyl)-piperidine (J) or (J′) is initially present in or added to the solvent in a total amount of from 0.4 to 0.6 mol/L.

In one embodiment of the third aspect of the invention, the N-protected-4-piperidinesulfonamide (K) or (K′) is isolated by crystallisation.

In one embodiment of the second or the third aspect of the invention, at the end of the reaction the process of step (v) further comprises the work-up steps of:
(3) optionally removing a portion of the solvent from the reaction mixture, e.g. by distillation;
(4) optionally diluting the remaining reaction mixture after step (3) with a second solvent;
(5) washing the resultant mixture with an aqueous wash and separating the organic and aqueous phases;
(6) optionally removing a portion of the solvent from the organic phase, e.g. by distillation;
(7) crystallising the N-protected-4-piperidinesulfonamide (K) or (K′) from the organic phase; and
(8) optionally isolating the crystalline N-protected-4-piperidinesulfonamide (K) or (K′), e.g. by filtration.

Typically, steps (3), (4), (6) and (8) are not optional.

Where a portion of the solvent is removed in step (3), typically about 50-75% of the solvent by volume is removed.

The second solvent in step (4) is typically a polar aprotic solvent such as dimethyl sulfoxide, N,N-dimethylformamide, N,N′-dimethylpropyleneurea, tetrahydrofuran, 1,4-dioxane, ethyl acetate, isopropyl acetate, acetone, acetonitrile, dichloromethane, hexamethylphosphoramide, nitromethane, propylene carbonate, N-methyl pyrrolidone, or a mixture thereof. Typically, the second solvent is a C₁-C₆ alkyl acetate, such as ethyl acetate, n-propyl acetate or isopropyl acetate. Most typically, the second solvent is isopropyl acetate.

Typically, water is used as the aqueous wash in step (5).

Optionally in step (6), a further portion of the second solvent is introduced as the portion of the solvent is removed by distillation. Typically in such an embodiment, an approximately constant volume of the organic phase is maintained.

The crystallisation of step (7) may be induced by cooling, e.g. from a temperature of 40 to 80° C. to a temperature of 0 to 30° C., and/or by the use of an antisolvent such as water. Optionally, the crystallisation of step (7) may be induced by seeding the organic phase with crystals of the N-protected-4-piperidinesulfonamide (K) or (K′).

Typically after the crystalline N-protected-4-piperidinesulfonamide (K) or (K′) is isolated by filtration in step (8), the crystalline N-protected-4-piperidinesulfonamide (K) or (K′) is washed, e.g. with water and/or isopropyl acetate, and dried under vacuum.

In one embodiment of the third aspect of the invention, the process comprises at least step (vi).

Typically, where the process of the third aspect of the invention comprises step (vi), the N-protected-4-piperidinesulfonamide (K) or (K′) is combined with the reaction mixture of step (vi) in non-salt form.

Typically, where the process of the third aspect of the invention comprises step (vi), the 1-ethyl-4-piperadinesulfonamide (A) is obtained in step (vi) in non-salt form.

Where the process of the third aspect of the invention comprises step (vi), typically the C₃-C₅ alcohol is selected from n-propanol, isopropanol, n-butanol, sec-butanol, iso-butanol, tert-butanol, tert-amyl alcohol, or any mixture thereof. More typically, the C₃-C₅ alcohol is selected from n-propanol, isopropanol, n-butanol, sec-butanol, iso-butanol or tert-butanol. Most typically, the C₃-C₅ alcohol is a butanol such as n-butanol.

Typically in accordance with the third aspect of the invention, R² is a nitrogen protecting group that may be removed by catalytic hydrogenolysis.

In one embodiment of the third aspect of the invention, where R² is a nitrogen protecting group that may be removed by catalytic hydrogenolysis, the reaction step (vi) comprises contacting the N-protected-4-piperidinesulfonamide (K) with acetonitrile or acetaldehyde in the presence of a catalyst, hydrogen gas and the C₃-C₅ alcohol, to obtain 1-ethyl-4-piperadinesulfonamide (A). Typically in such an embodiment, the reaction step (f) comprises contacting the N-protected-4-piperidinesulfonamide (K) with acetonitrile in the presence of a catalyst, hydrogen gas and the C₃-C₅ alcohol. Suitable catalysts include Raney nickel and palladium catalysts. In one embodiment, the catalyst is a palladium catalyst, for example palladium on carbon or palladium hydroxide on carbon. Typically, the catalyst is palladium on carbon.

Where the catalyst in step (vi) is palladium on carbon or palladium hydroxide on carbon, typically from 2 to 35 wt. % palladium or palladium hydroxide on carbon is used. More typically, from 5 to 30 wt. % palladium or palladium hydroxide on carbon is used. Most typically, from 5 to 15 wt. % palladium or palladium hydroxide on carbon is used.

In an exemplary embodiment of the third aspect of the invention, the reaction step (vi) comprises contacting 1-(benzyloxycarbonyl)-4-piperidinesulfonamide (K′) with acetonitrile or acetaldehyde in the presence of a catalyst, hydrogen gas, and a C₃-C₅ alcohol, to obtain 1-ethyl-4-piperadinesulfonamide (A):

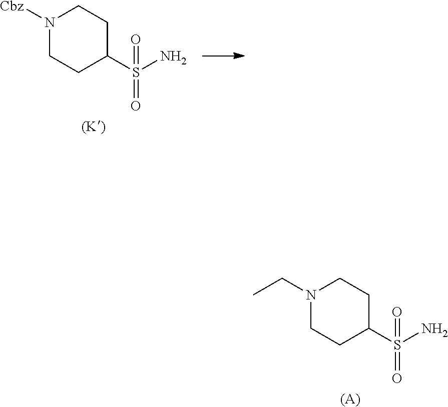

Typically in such an embodiment, the 1-(benzyloxycarbonyl)-4-piperidinesulfonamide (K′) is contacted with acetonitrile in the presence of a catalyst, hydrogen gas and a butanol such as n-butanol. Typically the catalyst is a palladium catalyst such as palladium on carbon.

Where reaction step (vi) of the third aspect of the invention comprises contacting the N-protected-4-piperidinesulfonamide (K) or (K′) with acetonitrile or acetaldehyde in the presence of a catalyst and hydrogen gas, typically the hydrogen gas is used at a pressure in the range from 0.1 to 100 Bar. In one embodiment, the hydrogen gas is used at a pressure in the range from 0.5 to 50 Bar, and more typically in the range from 5 to 25 Bar.

In one embodiment of the third aspect of the invention, the N-protected-4-piperidinesulfonamide (K) or (K′) is contacted with acetonitrile or acetaldehyde in the presence of a catalyst, hydrogen gas, the C₃-C₅ alcohol and water. Typically, where the contact takes place in the presence of water and a C₃-C₅ alcohol, the ratio of water:C₃-C₅ alcohol present is from 1:1 to 1:10 by volume. More typically, the ratio of water:C₃-C₅ alcohol is from 1:2 to 1:5 by volume. Typically in such an embodiment, the N-protected-4-piperidinesulfonamide (K) or (K′) is contacted with acetonitrile in the presence of a catalyst, hydrogen gas, the C₃-C₅ alcohol and water.

Where reaction step (vi) of the third aspect of the invention comprises contacting the N-protected-4-piperidinesulfonamide (K) or (K′) with acetonitrile or acetaldehyde in the presence of a catalyst and hydrogen gas, typically the reaction step (vi) is carried out at a temperature in the range from 0 to 100° C. In one embodiment, the reaction step (vi) is carried out at a temperature in the range from 15 to 80° C. Typically in such an embodiment, the reaction step (vi) is carried out at a temperature in the range from 20 to 70° C. More typically in such an embodiment, the reaction step (vi) is carried out at a temperature in the range from 55 to 65° C.

The process of the third aspect of the invention may comprise at least two of steps (i)-(vi).

Accordingly, in one embodiment of the third aspect of the invention, the process comprises at least steps (i) and (ii). Typically in any embodiment of the third aspect of the invention that comprises steps (i) and (ii), the N-protected-4-hydroxy piperidine (G) or (G′) is not isolated between steps (i) and (ii).

In another embodiment of the third aspect of the invention, the process comprises at least steps (ii) and (iii). Typically in any embodiment of the third aspect of the invention that comprises steps (ii) and (iii), the N-protected-4-derivatised piperidine (H) or (H′) is not isolated between steps (ii) and (iii).

In a further embodiment of the third aspect of the invention, the process comprises at least steps (iii) and (iv). Typically in any embodiment of the third aspect of the invention that comprises steps (iii) and (iv), the thiourea adduct (I) or (I′) is isolated between steps (iii) and (iv). Typically, where the thiourea adduct (I) or (I′) is isolated it is isolated in salt form, e.g. as a sulfonic acid addition salt such as a methanesulfonic acid salt.

In yet another embodiment of the third aspect of the invention, the process comprises at least steps (iv) and (v). Typically in any embodiment of the third aspect of the invention that comprises steps (iv) and (v), the N-protected-4-(halosulfonyl)-piperidine (J) or (J′) is isolated between steps (iv) and (v). Typically, where the N-protected-4-(halosulfonyl)-piperidine (J) or (J′) is isolated it is isolated in non-salt form.

In a further embodiment of the third aspect of the invention, the process comprises at least steps (v) and (vi). Typically in any embodiment of the third aspect of the invention that comprises steps (v) and (vi), the N-protected-4-piperidinesulfonamide (K) or (K′) is isolated between steps (v) and (vi). Typically, where the N-protected-4-piperidinesulfonamide (K) or (K′) is isolated it is isolated in non-salt form.

The process of the third aspect of the invention may comprise at least three of steps (i)-(vi).

Accordingly, in one embodiment of the third aspect of the invention, the process comprises at least steps (i), (ii) and (iii).

In another embodiment of the third aspect of the invention, the process comprises at least steps (ii), (iii) and (iv).

In a further embodiment of the third aspect of the invention, the process comprises at least steps (iii), (iv) and (v).

In yet another embodiment of the third aspect of the invention, the process comprises at least steps (iv), (v) and (vi).

The process of the third aspect of the invention may comprise at least four of steps (i)-(vi).

Accordingly, in one embodiment of the third aspect of the invention, the process comprises at least steps (i), (ii), (iii) and (iv).

In another embodiment of the third aspect of the invention, the process comprises at least steps (ii), (iii), (iv) and (v).

In a further embodiment of the third aspect of the invention, the process comprises at least steps (iii), (iv), (v) and (vi).

The process of the third aspect of the invention may comprise at least five of steps (i)-(vi).

Accordingly, in one embodiment of the third aspect of the invention, the process comprises at least steps (i), (ii), (iii), (iv) and (v).

In another embodiment of the third aspect of the invention, the process comprises at least steps (ii), (iii), (iv), (v) and (vi).

Typically, the process of the third aspect of the invention comprises all six of steps (i)-(vi).

Typically, the process of any of the first, second or third aspects of the invention is a process for the preparation of 1-ethyl-4-piperadinesulfonamide (A) or a salt thereof. Such a process may comprise the conversion of any of the N-protected-4-hydroxy piperidine (G) or (G′), the N-protected-4-derivatised piperidine (H) or (H′), the thiourea adduct (I) or (I′), the N-protected-4-(halosulfonyl)-piperidine (J) or (J′), the N-protected-4-piperidinesulfonamide (K) or (K′), or any of the salts thereof, into 1-ethyl-4-piperadinesulfonamide (A) or a salt thereof.

In one embodiment of any of the first, second or third aspects of the invention, the process is for the preparation of 1-ethyl-N-((1,2,3,5,6,7-hexahydro-s-indacen-4-yl)carbamoyl)piperidine-4-sulfonamide or a salt thereof. Such a process may comprise the conversion of any of the N-protected-4-hydroxy piperidine (G) or (G′), the N-protected-4-derivatised piperidine (H) or (H′), the thiourea adduct (I) or (I′), the N-protected-4-(halosulfonyl)-piperidine (J) or (J′), the N-protected-4-piperidinesulfonamide (K) or (K′), the 1-ethyl-4-piperadinesulfonamide (A), or any of the salts thereof, into 1-ethyl-N-((1,2,3,5,6,7-hexahydro-s-indacen-4-yl)carbamoyl)piperidine-4-sulfonamide or a salt thereof. Typically, such a process comprises the step of contacting 1-ethyl-4-piperidinesulfonamide (A) (as prepared in accordance with any of the first to third aspects of the invention) with a 1,2,3,5,6,7-hexahydro-s-indacene derivative (B) in the presence of a solvent to obtain 1-ethyl-N-((1,2,3,5,6,7-hexahydro-s-indacen-4-yl)carbamoyl)piperidine-4-sulfonamide (C) or a salt thereof:

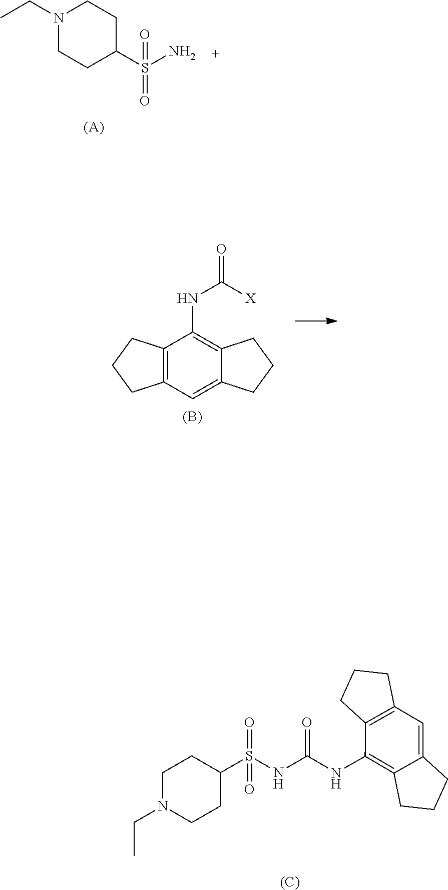

wherein X is a leaving group.

In one embodiment, X is Cl, Br, I, OR¹, SR¹, N(R¹)₂, OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen or phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups RX, wherein each RX is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

In one embodiment, X is Cl, Br or I. Typically in such an embodiment, X is Cl.

In another embodiment, X is OR¹ or SR¹, wherein R¹ is a C₁-C₂₀ hydrocarbyl group, wherein the C₁-C₂₀ hydrocarbyl group may be straight-chained or branched, or be or include one or more cyclic groups, wherein the C₁-C₂₀ hydrocarbyl group may optionally be substituted with one or more oxo (═O) and/or one or more halo groups, and wherein the C₁-C₂₀ hydrocarbyl group may optionally include one or more heteroatoms independently selected from N, O and S in its carbon skeleton.

Typically in such an embodiment, X is OR¹.

For example, X may be OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), ═NH, —R¹⁰, —OR¹⁰, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X is OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

In one embodiment, X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X is OR¹, wherein R¹ is selected from a phenyl or a monocyclic heteroaryl group, wherein R¹ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

More typically still, X is OR¹, wherein R¹ is a phenyl group, wherein the phenyl group is optionally substituted with one or more fluoro, chloro or —NO₂ groups. Most typically, R¹ is an unsubstituted phenyl group, i.e. X is OPh.

When R¹ is an unsubstituted phenyl group, there is provided a process of preparing 1-ethyl-N-((1,2,3,5,6,7-hexahydro-s-indacen-4-yl)carbamoyl)piperidine-4-sulfonamide or a salt thereof, comprising the step of contacting the 1-ethyl-4-piperidinesulfonamide (A) (as prepared in accordance with any of the first to third aspects of the invention) with 4-(phenoxycarbonylamino)-1,2,3,5,6,7-hexahydro-s-indacene (B′) in the presence of a solvent to obtain 1-ethyl-N-((1,2,3,5,6,7-hexahydro-s-indacen-4-yl)carbamoyl)-piperidine-4-sulfonamide (C) or a salt thereof:

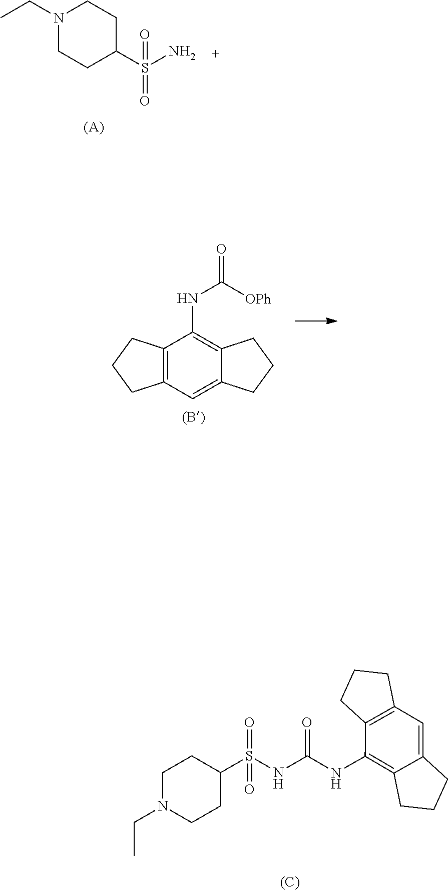

In another embodiment, X is N(R¹)₂, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the nitrogen atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups RX, wherein each RX is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically in such an embodiment, X is N(R¹)₂, wherein the two R¹ together with the nitrogen atom to which they are attached form a 5- to 14-membered heteroaryl group, wherein the heteroaryl group may be monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, where X is N(R¹)₂, the two R¹ together with the nitrogen atom to which they are attached form a 5- to 10-membered heteroaryl group, wherein the heteroaryl group may be monocyclic or bicyclic, wherein R¹ may optionally be substituted with one or more substituents independently selected from halo, —CN, —OH, —NO₂, —NH₂, -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein R¹, including any optional substituents, contains from 1 to 12 carbon atoms.

Typically, where X is N(R¹)₂ and the two R¹ together with the nitrogen atom to which they are attached form a 5- to 14- or 5- to 10-membered heteroaryl group, the ring that encompasses the nitrogen atom of N(R¹)₂ is a 5-membered ring.

In another embodiment, X is OP(═O)(R¹)₂ or OP(R¹)₃⁺, wherein each R¹ is independently selected from a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two R¹ together with the phosphorus atom to which they are attached may form a 3- to 16-membered heterocyclic group, wherein the heterocyclic group may be monocyclic, bicyclic or tricyclic, and wherein the heterocyclic group may optionally be substituted with one or more halo groups and/or one or more groups RX, wherein each RX is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

Typically in such an embodiment, X is OP(═O)(R¹)₂ or OP(R¹)₃⁺, wherein each R¹ is independently selected from an alkyl, cycloalkyl, aryl, heteroaryl, arylalkyl or heteroarylalkyl group, wherein each R¹ may optionally be substituted with one or more substituents independently selected from halo, —CN, —OH, —NO₂, —NH₂, oxo (═O), ═NH, —R¹⁰, —OR¹⁰, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein each R¹, including any optional substituents, contains from 1 to 20 carbon atoms.

More typically, where X is OP(═O)(R¹)₂ or OP(R¹)₃⁺, each R¹ is independently selected from an alkyl, cycloalkyl, aryl, heteroaryl, arylalkyl or heteroarylalkyl group, wherein each R¹ may optionally be substituted with one or more substituents independently selected from halo, —CN, —OH, —NO₂, —NH₂, oxo (═O), -Me, -Et, —OMe, —OEt, —NHMe, —NHEt, —N(Me)₂, —N(Me)Et or —N(Et)₂, wherein any methyl (Me) or ethyl (Et) group may optionally be substituted with one or more halo groups, and wherein each R¹, including any optional substituents, contains from 1 to 12 carbon atoms.

More typically still, where X is OP(═O)(R¹)₂ or OP(R)₃⁺, each R¹ is independently selected from a C₁-C₄ alkyl or phenyl group.

Typically, where the process comprises the step of contacting the 1-ethyl-4-piperidinesulfonamide (A) with the 1,2,3,5,6,7-hexahydro-s-indacene derivative (B) or (B′), the 1-ethyl-4-piperidinesulfonamide (A) is combined with the reaction mixture in non-salt form.

Typically, where the process comprises the step of contacting the 1-ethyl-4-piperidinesulfonamide (A) with the 1,2,3,5,6,7-hexahydro-s-indacene derivative (B) or (B′), the 1,2,3,5,6,7-hexahydro-s-indacene derivative (B) or (B′) is combined with the reaction mixture in non-salt form.

In one embodiment, the step of contacting the 1-ethyl-4-piperidinesulfonamide (A) with the 1,2,3,5,6,7-hexahydro-s-indacene derivative (B) or (B′) is performed in the presence of a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group. Typically the solvent is not halogenated. For example, the solvent may be selected from dimethyl sulfoxide, tetrahydrofuran, 1,4-dioxane, acetonitrile, hexamethylphosphoramide, nitromethane, or a mixture thereof. More typically still, the solvent does not comprise a carbonyl, C═N or C≡N group. Typically, where the solvent does not comprise a carbonyl, C═N or C≡N group, the solvent is not halogenated. For example, the solvent may be selected from dimethyl sulfoxide, tetrahydrofuran, 1,4-dioxane, hexamethylphosphoramide, nitromethane, or a mixture thereof. Most typically, the solvent is dimethyl sulfoxide.

In one embodiment, the step of contacting the 1-ethyl-4-piperidinesulfonamide (A) with the 1,2,3,5,6,7-hexahydro-s-indacene derivative (B) or (B′) is performed in the presence of a base. Typically the base is an alkoxide base, such as an alkali metal or an alkali earth metal alkoxide. More typically the base is a tertiary butoxide base such as an alkali metal or an alkali earth metal tertiary butoxide. Examples of suitable bases include potassium tertiary butoxide and sodium tertiary butoxide. Typically, the base is potassium tertiary butoxide.

Typically, the 1-ethyl-N-((1,2,3,5,6,7-hexahydro-s-indacen-4-yl)carbamoyl)-piperidine-4-sulfonamide (C) is obtained in salt form.

Accordingly, one embodiment of any of the first, second or third aspects of the invention provides a process of preparing a salt of 1-ethyl-N-((1,2,3,5,6,7-hexahydro-s-indacen-4-yl)carbamoyl)piperidine-4-sulfonamide, such as a cationic salt. Typically the salt is pharmaceutically acceptable.

For the purposes of this invention, a “cationic salt” of 1-ethyl-N-((1,2,3,5,6,7-hexahydro-s-indacen-4-yl)carbamoyl)piperidine-4-sulfonamide is a salt formed between a protic acid functionality (such as a urea proton) of the compound by the loss of a proton and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt. More preferably the salt is a mono- or di-potassium salt, more preferably still the salt is a mono-potassium salt.

Advantageously, where a cationic salt of 1-ethyl-N-((1,2,3,5,6,7-hexahydro-s-indacen-4-yl)carbamoyl)piperidine-4-sulfonamide (C) is desired, the cation of the salt is provided by the conjugate acid of the base. For example, in one embodiment there is provided a process of preparing an alkali metal or an alkali earth metal salt of 1-ethyl-N-((1,2,3,5,6,7-hexahydro-s-indacen-4-yl)carbamoyl)piperidine-4-sulfonamide (C), comprising the step of contacting the 1-ethyl-4-piperidinesulfonamide (A) with the 1,2,3,5,6,7-hexahydro-s-indacene derivative (B) or (B′) in the presence of a solvent and an alkali metal or an alkali earth metal alkoxide, to obtain the alkali metal or alkali earth metal salt of 1-ethyl-N-((1,2,3,5,6,7-hexahydro-s-indacen-4-yl)-carbamoyl)-piperidine-4-sulfonamide, wherein the alkali metal or alkali earth metal of the salt is the same as the alkali metal or alkali earth metal of the alkoxide. Typically in such an embodiment, the alkali metal or alkali earth metal alkoxide is an alkali metal or an alkali earth metal tertiary butoxide.

A further embodiment provides a process of preparing a potassium salt of 1-ethyl-N-((1,2,3,5,6,7-hexahydro-s-indacen-4-yl)carbamoyl)piperidine-4-sulfonamide (C), the process comprising the step of contacting the 1-ethyl-4-piperidinesulfonamide (A) with the 4-(phenoxycarbonylamino)-1,2,3,5,6,7-hexahydro-s-indacene (B′) in the presence of a solvent and potassium tertiary butoxide, to obtain the potassium salt of 1-ethyl-N-((1,2,3,5,6,7-hexahydro-s-indacen-4-yl)carbamoyl)-piperidine-4-sulfonamide. Typically in such an embodiment, the potassium salt is a mono-potassium salt.

In one embodiment, the step of contacting the 1-ethyl-4-piperidinesulfonamide (A) with the 1,2,3,5,6,7-hexahydro-s-indacene derivative (B) or (B′) to obtain the 1-ethyl-N-((1,2,3,5,6,7-hexahydro-s-indacen-4-yl)carbamoyl)piperidine-4-sulfonamide (C) or the salt thereof, is carried out at a temperature in the range from −10 to 60° C. Typically, the step is carried out at a temperature in the range from 0 to 50° C., more typically in the range from 10 to 40° C., and most typically in the range from 20 to 30° C.

Typically, where the process comprises the step of contacting the 1-ethyl-4-piperidinesulfonamide (A) with the 1,2,3,5,6,7-hexahydro-s-indacene derivative (B) or (B′) the 1-ethyl-4-piperidine-sulfonamide (A) is present in or added to the solvent at an initial concentration of from 0.1 to 15 mol/L, relative to the total volume of solvent used in the reaction mixture. More typically, 1-ethyl-4-piperidinesulfonamide (A) is present in or added to the solvent at an initial concentration of from 0.5 to 5.0 mol/L. Most typically, the 1-ethyl-4-piperidinesulfonamide (A) is present in or added to the solvent at an initial concentration of from 1.0 to 1.5 mol/L. Typically, the 1,2,3,5,6,7-hexahydro-s-indacene derivative (B) or (B′) is present in or added to the solvent at an initial concentration of from 0.1 to 15 mol/L, relative to the total volume of solvent used in the reaction mixture. More typically, the 1,2,3,5,6,7-hexahydro-s-indacene derivative (B) or (B′) is present in or added to the solvent at an initial concentration of from 0.5 to 5.0 mol/L. Most typically, the 1,2,3,5,6,7-hexahydro-s-indacene derivative (B) or (B′) is present in or added to the solvent at an initial concentration of from 1.0 to 1.5 mol/L.

Typically, where the process comprises the step of contacting the 1-ethyl-4-piperidinesulfonamide (A) with the 1,2,3,5,6,7-hexahydro-s-indacene derivative (B) or (B′), the process uses from 0.8 to 1.4 molar equivalents of the 1,2,3,5,6,7-hexahydro-s-indacene derivative (B) or (B′) relative to the initial amount of 1-ethyl-4-piperidinesulfonamide (A). More typically, the process uses from 1.0 to 1.2 molar equivalents of the 1,2,3,5,6,7-hexahydro-s-indacene derivative (B) or (B′). Most typically, the process uses from 1.05 to 1.15 molar equivalents of the 1,2,3,5,6,7-hexahydro-s-indacene derivative (B) or (B′).

Typically, where the process comprises the step of contacting the 1-ethyl-4-piperidinesulfonamide (A) with the 1,2,3,5,6,7-hexahydro-s-indacene derivative (B) or (B′) in the presence of a base, the process uses from 1.0 to 2.0 molar equivalents of the base, relative to the initial amount of the 1-ethyl-4-piperidinesulfonamide (A). More typically, the process uses from 1.05 to 1.5 molar equivalents of the base. More typically still, the process uses from 1.1 to 1.2 molar equivalents of the base.

In one embodiment, the process comprises the steps of:
(1) dissolving the 1-ethyl-4-piperidinesulfonamide (A) in the solvent;
(2) adding the base to the solution formed in step (1); and
(3) adding the 1,2,3,5,6,7-hexahydro-s-indacene derivative (B) or (B′) to the mixture formed in step (2).

In one embodiment, the 1-ethyl-N-((1,2,3,5,6,7-hexahydro-s-indacen-4-yl)carbamoyl)piperidine-4-sulfonamide (C) or the salt thereof is isolated from the reaction mixture by crystallisation or precipitation. For example, where the solvent used in the reaction is dimethyl sulfoxide (DMSO), further solvents such as water, acetonitrile (MeCN) and optionally further DMSO may be added to the reaction mixture to create a precipitation mixture from which the 1-ethyl-N-((1,2,3,5,6,7-hexahydro-s-indacen-4-yl)carbamoyl)piperidine-4-sulfonamide (C) or the salt thereof is precipitated, optionally under cooling. In one embodiment, the precipitation mixture comprises DMSO, MeCN and water, wherein the solvent of the precipitation mixture consists of 30-50 wt. % DMSO (relative to the total weight of the solvent), 50-70 wt. % MeCN (relative to the total weight of the solvent), and 1-10 wt. % H₂O (relative to the total weight of the solvent). Typically the crystallisation or precipitation occurs at a temperature in the range from −10 to 20° C. More typically, the crystallisation or precipitation occurs at a temperature in the range from −5 to 10° C., and most typically in the range from 0 to 5° C. Typically, a salt of 1-ethyl-N-((1,2,3,5,6,7-hexahydro-s-indacen-4-yl)carbamoyl)piperidine-4-sulfonamide (C) is isolated from the reaction mixture by crystallisation or precipitation. Typically the salt is an alkali metal or alkali earth metal salt, such as a potassium salt.

In one embodiment, the isolated salt of 1-ethyl-N-((1,2,3,5,6,7-hexahydro-s-indacen-4-yl)carbamoyl)piperidine-4-sulfonamide (C) is further purified by recrystallisation or reprecipitation. For example, the isolated salt of 1-ethyl-N-((1,2,3,5,6,7-hexahydro-s-indacen-4-yl)carbamoyl)piperidine-4-sulfonamide (C) may be dissolved in a first solvent to a obtain a first mixture, optionally the mixture may be filtered, and the salt of the 1-ethyl-N-((1,2,3,5,6,7-hexahydro-s-indacen-4-yl)-carbamoyl)piperidine-4-sulfonamide (C) may be precipitated by the addition of a second solvent, optionally with cooling. Typically, the first solvent is a polar protic solvent such as methanol. Typically, the second solvent is a polar aprotic solvent such as acetonitrile.

Where the process of the invention comprises the use of 1,2,3,5,6,7-hexahydro-s-indacene derivative (B), the 1,2,3,5,6,7-hexahydro-s-indacene derivative (B) may be prepared by a process comprising the step of:
(e) converting 1,2,3,5,6,7-hexahydro-s-indacen-4-amine (D) to the 1,2,3,5,6,7-hexahydro-s-indacene derivative (B):

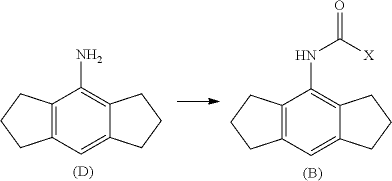

wherein X is as defined above.

In one embodiment, such a process comprises the step of contacting the 1,2,3,5,6,7-hexahydro-s-indacen-4-amine (D) with reagent (E):

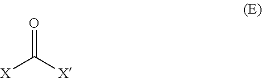

optionally in the presence of a base and/or a solvent, wherein X is as defined above and X′ is a leaving group.

In one embodiment, X′ is Cl, Br, I, OR¹, SR¹, N(R¹)₂, OP(═O)(R¹)₂ or OP(R¹)₃⁺, wherein each R¹ is as defined above. Typically, X′ is Cl, Br or I. More typically, X′ is Cl or Br. Most typically, X′ is Cl.

X and X′ may be the same or different. Typically X and X′ are different. Typically X and X are selected such that X′ is more readily displaced than X.

In one embodiment, X′ is Cl, Br or I, and X is OR¹, SR¹, N(R¹)₂, OP(═O)(R¹)₂ or OP(R¹)₃⁺. More typically, X′ is Cl or Br, and X is OR¹, SR¹ or N(R¹)₂.

In a further embodiment, X′ is Cl, Br or I, and X is OR¹, wherein R¹ is selected from an alkyl, cycloalkyl, aryl, heteroaryl, arylalkyl or heteroarylalkyl group, wherein R¹ may optionally be substituted with one or more substituents independently selected from halo, —CN, —OH, —NO₂, —NH₂, oxo (═O), ═NH, —R¹⁰, —OR¹⁰, —NHR¹⁰, —N(R¹⁰)₂, —N(O)(R¹⁰)₂, or ═NR¹⁰, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically, X′ is Cl or Br, and X is OR¹, wherein R¹ is selected from an aryl or heteroaryl group, wherein the aryl or heteroaryl group is monocyclic, bicyclic or tricyclic, wherein R¹ may optionally be substituted with one or more substituents independently selected from halo, —CN, —OH, —NO₂, —NH₂, —R¹⁰, —OR¹⁰, —NHR¹⁰, —N(R¹⁰)₂ or —N(O)(R¹⁰)₂, wherein each R¹⁰ is independently selected from a C₁-C₄ alkyl, C₁-C₄ haloalkyl, C₃-C₄ cycloalkyl or C₃-C₄ halocycloalkyl group, or any two R¹⁰ directly attached to the same nitrogen atom may together form a C₂-C₅ alkylene or C₂-C₅ haloalkylene group, and wherein R¹, including any optional substituents, contains from 1 to 20 carbon atoms.

More typically still, X′ is Cl and X is OR¹, wherein R¹ is a phenyl group, wherein the phenyl group is optionally substituted with one or more fluoro, chloro or —NO₂ groups. Most typically, X′ is Cl and X is OPh.

Accordingly, in an exemplary embodiment, the process of step (e) comprises contacting the 1,2,3,5,6,7-hexahydro-s-indacen-4-amine (D) with phenyl chloroformate (E′), optionally in the presence of a solvent and/or a base:

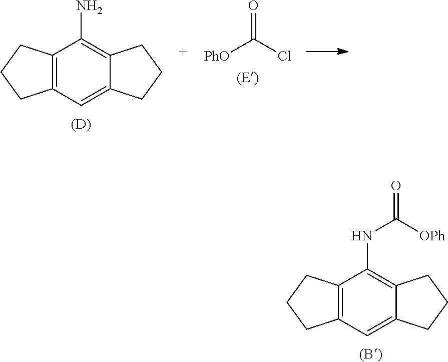

Typically, the 1,2,3,5,6,7-hexahydro-s-indacen-4-amine (D) is contacted with reagent (E) or (E′) in the presence of a solvent. In one embodiment, the solvent is a polar aprotic solvent such as dimethyl sulfoxide, N,N-dimethylformamide, N,N′-dimethylpropyleneurea, tetrahydrofuran, 1,4-dioxane, ethyl acetate, acetone, acetonitrile, dichloromethane, hexamethylphosphoramide, nitromethane, propylene carbonate, N-methyl pyrrolidone, or a mixture thereof. Typically the solvent does not comprise an ester. More typically the solvent does not comprise a carbonyl group.

Typically the solvent is not halogenated. For example, the solvent may be selected from dimethyl sulfoxide, tetrahydrofuran, 1,4-dioxane, acetonitrile, hexamethylphosphoramide, nitromethane, or a mixture thereof. More typically still, the solvent does not comprise a carbonyl, C═N or C≡N group. Typically, where the solvent does not comprise a carbonyl, C═N or C≡N group, the solvent is not halogenated. For example, the solvent may be selected from dimethyl sulfoxide, tetrahydrofuran, 1,4-dioxane, hexamethylphosphoramide, nitromethane, or a mixture thereof. Most typically, the solvent is tetrahydrofuran.

Typically, the 1,2,3,5,6,7-hexahydro-s-indacen-4-amine (D) is contacted with reagent (E) or (E′) in the presence of a base. Typically, the base is a sterically hindered base. For example, the base may be a tertiary amine such as N,N-diisopropylethylamine (DIPEA), trimethylamine, triethylamine (TEA), tripropylamine or tributylamine. Most typically, the base is N,N-diisopropylethylamine.

Typically, where the process of the invention comprises step (e), the 1,2,3,5,6,7-hexahydro-s-indacen-4-amine (D) is combined with the reaction mixture of step (e) in non-salt form.

Typically, where the process of the invention comprises step (e), the reagent (E) or (E′) is combined with the reaction mixture of step (e) in non-salt form.

Typically, where the process of the invention comprises step (e), the 1,2,3,5,6,7-hexahydro-s-indacene derivative (B) or (B′) is obtained in step (e) in non-salt form.

In one embodiment, the 1,2,3,5,6,7-hexahydro-s-indacen-4-amine (D) is combined with reagent (E) or (E′) at a temperature in the range from −10 to 40° C. Typically, the 1,2,3,5,6,7-hexahydro-s-indacen-4-amine (D) is combined with reagent (E) or (E′) at a temperature in the range from 0 to 25° C., more typically in the range from 0 to 10° C. In one embodiment, after the 1,2,3,5,6,7-hexahydro-s-indacen-4-amine (D) has been combined with reagent (E) or (E′), the reaction mixture is allowed to warm to a temperature in the range from 5 to 50° C. Typically, the reaction mixture is allowed to warm to a temperature in the range from 10 to 30° C., more typically in the range from to 25° C.

Typically, where the process comprises the step of contacting the 1,2,3,5,6,7-hexahydro-s-indacen-4-amine (D) with reagent (E) or (E′) in the presence of a solvent, the 1,2,3,5,6,7-hexahydro-s-indacen-4-amine (D) is present in or added to the solvent at an initial concentration of from 0.01 to 10 mol/L relative to the total volume of solvent used in the reaction mixture. More typically, the 1,2,3,5,6,7-hexahydro-s-indacen-4-amine (D) is present in or added to the solvent at an initial concentration of from 0.1 to 1.0 mol/L. Most typically the 1,2,3,5,6,7-hexahydro-s-indacen-4-amine (D) is present in or added to the solvent at an initial concentration of from 0.4 to 0.5 mol/L.

Typically, the process uses from 0.9 to 1.5 molar equivalents of reagent (E) or (E′), relative to the initial amount of 1,2,3,5,6,7-hexahydro-s-indacen-4-amine (D). More typically, the process uses from 1.0 to 1.2 molar equivalents of the reagent (E) or (E′). Most typically, the process uses from 1.05 to 1.15 molar equivalents of reagent (E) or (E′).

Typically, where the process comprises the step of contacting the 1,2,3,5,6,7-hexahydro-s-indacen-4-amine (D) with reagent (E) or (E′) in the presence of a base, the process uses from 0.8 to 2.0 molar equivalents of the base, relative to the initial amount 1,2,3,5,6,7-hexahydro-s-indacen-4-amine (D). More typically, the process uses from 1.0 to 1.5 molar equivalents of the base. Most typically, the process uses from 1.1 to 1.3 molar equivalents of the base.

In one embodiment, the process of step (e) comprises the steps of:
(1) dissolving the 1,2,3,5,6,7-hexahydro-s-indacen-4-amine (D) in a first portion of the solvent;
(2) dissolving the base in a second portion of the solvent and adding the resultant solution to the solution formed in step (1); and
(3) dissolving reagent (E) or (E′) in a third portion of the solvent and adding the resultant solution to the mixture formed in step (2).

In one embodiment, at the end of the reaction between the 1,2,3,5,6,7-hexahydro-s-indacen-4-amine (D) and the reagent (E) or (E′), the process further comprises the steps of:
(4) concentrating the reaction mixture under vacuum; then
(5) optionally adding a co-solvent and concentrating the resultant mixture under vacuum.

Step (5) may be repeated one or more times. Typically the co-solvent is an alcohol such as methanol or ethanol. Most typically the co-solvent is ethanol.

In one embodiment, the 1,2,3,5,6,7-hexahydro-s-indacene derivative (B) is purified and/or isolated by crystallisation or precipitation. For example, a precipitation solvent may be added to the concentrated reaction mixture following step (4) or (5) above to create a precipitation mixture from which the 1,2,3,5,6,7-hexahydro-s-indacene derivative (B) may be precipitated, optionally under cooling. Typically the crystallisation or precipitation occurs at a temperature in the range from −10 to 20° C. More typically, the crystallisation or precipitation occurs at a temperature in the range from −5 to 10° C., and most typically in the range from 0 to 5° C. Typically the precipitation solvent is an alcohol such as methanol or ethanol. Most typically the precipitation solvent is ethanol.

Typically, a non-salt form of the 1,2,3,5,6,7-hexahydro-s-indacene derivative (B) is isolated by crystallisation or precipitation. Most typically, a non-salt form of 4-(phenoxycarbonylamino)-1,2,3,5,6,7-hexahydro-s-indacene (B′) is isolated by crystallisation or precipitation.

In one embodiment, where a process of the invention comprises the use of 1,2,3,5,6,7-hexahydro-s-indacen-4-amine (D), the 1,2,3,5,6,7-hexahydro-s-indacen-4-amine (D) is prepared by a process comprising one or more steps selected from:
(a) contacting 2,3-dihydro-1H-indene (L) with YCH₂CH₂C(O)Z (M) to obtain a substituted 1-(2,3-dihydro-1H-inden-5-yl)propan-1-one (N), wherein Y and Z are leaving groups:

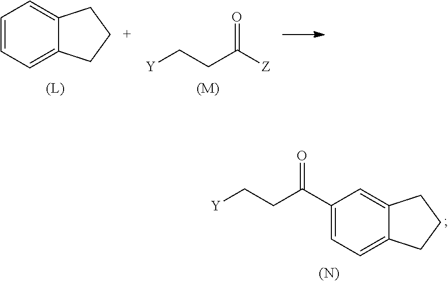

(b) contacting the substituted 1-(2,3-dihydro-1H-inden-5-yl)propan-1-one (N) with an acid to obtain 1,2,3,5,6,7-hexahydro-s-indacen-1-one (P):

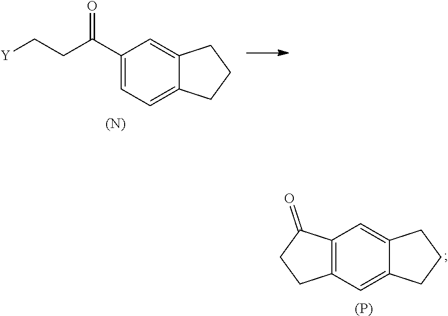

(c) converting 1,2,3,5,6,7-hexahydro-s-indacen-1-one (P) to 8-nitro-1,2,3,5,6,7-hexahydro-s-indacen-1-one (Qa) and/or 4-nitro-1,2,3,5,6,7-hexahydro-s-indacen-1-one (Qb):

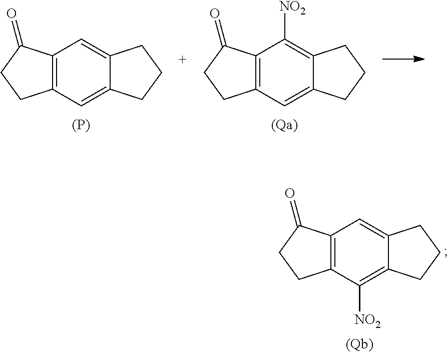

and
(d) reducing 8-nitro-1,2,3,5,6,7-hexahydro-s-indacen-1-one (Qa) and/or 4-nitro-1,2,3,5,6,7-hexahydro-s-indacen-1-one (Qb) to obtain 1,2,3,5,6,7-hexahydro-s-indacen-4-amine (D):

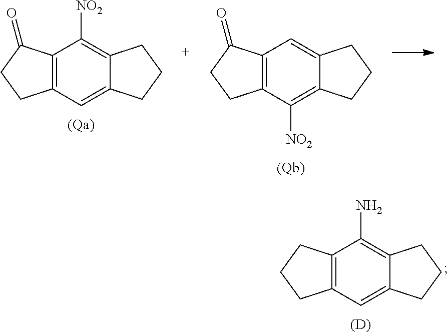

In one embodiment, the process comprises one, two, three or all four of steps (a) to (d).

The process for the preparation of 1,2,3,5,6,7-hexahydro-s-indacen-4-amine (D) may be as described in WO 2020/079207 A1, the contents of which are incorporated herein by reference in their entirety.

In one embodiment, in step (a), the leaving group Y is independently selected from Cl, Br, I, or a sulfonate leaving group such as a toluenesulfonate, methanesulfonate, or trifluoromethanesulfonate leaving group.

In one embodiment, in step (a), the leaving group Z is independently selected from Cl, Br, I, OR¹, SR¹, N(R¹)₂, OP(═O)(R¹)₂ or OP(R¹)₃⁺, wherein R¹ is as defined above.

Y and Z may be the same or different. Typically, Y and Z are each independently selected from Cl, Br and I. Typically, at least one of Y and Z is Cl. More typically, Y and Z are both Cl. When both Y and Z are Cl, in step (a) 2,3-dihydro-1H-indene (L) is contacted with 3-chloropropionyl chloride to obtain 3-chloro-1-(2,3-dihydro-1H-inden-5-yl)propan-1-one.

In one embodiment, the reaction of step (a) is carried out in the presence of a catalyst, such as a Lewis acid such as aluminium chloride.

Step (a) may be carried out in the presence of a solvent. In one embodiment, the solvent is an aprotic solvent. In one embodiment, the solvent is dichloromethane, dichloroethane, chloroform, diethyl ether, n-pentane, n-hexane, n-heptane, toluene, or a mixture thereof. Typically, the solvent is dichloromethane.

In one embodiment, the reaction of step (a) is carried out at a temperature in the range from −20 to 50° C. Typically, the reaction of step (a) is carried out at a temperature in the range from −15 to 25° C., more typically at a temperature in the range from −10 to 15° C.

In one embodiment, in step (b), the acid is sulfuric acid, hydrochloric acid, Eaton's reagent, polyphosphoric acid or a mixture thereof. Typically, the acid is sulfuric acid or hydrochloric acid. More typically, the acid is sulfuric acid. Typically, no additional solvent is used.

In one embodiment, the reaction of step (b) is carried out at a temperature in the range from 10 to 90° C. Typically, the reaction of step (b) is carried out at a temperature in the range from 40 to 80° C., more typically at a temperature in the range from 65 to 70° C.

In one embodiment, in step (c), 1,2,3,5,6,7-hexahydro-s-indacen-1-one (P) is converted to 8-nitro-1,2,3,5,6,7-hexahydro-s-indacen-1-one (Qa) or 4-nitro-1,2,3,5,6,7-hexahydro-s-indacen-1-one (Qb) or a mixture thereof by treatment with sulfuric acid and nitric acid. Typically, no additional solvent is used.

In one embodiment, the reaction of step (c) is carried out at a temperature in the range from 0 to 20° C. Typically, the reaction of step (c) is carried out at a temperature in the range from 0 to 10° C., more typically at a temperature in the range from 0 to 5° C.

In one embodiment, the reactions of steps (b) and (c) are carried out without isolating 1,2,3,5,6,7-hexahydro-s-indacen-1-one (P).

In one embodiment, the reduction of step (d) is carried out using a catalyst and hydrogen gas. Typically, the catalyst is a metal catalyst comprising platinum, palladium, rhodium, ruthenium or nickel. Typically, the catalyst is Pd/C, Pd(OH)₂/C, Pt/C, PtO₂, platinum black or Raney nickel. More typically, the catalyst is Pd/C or Pd(OH)₂/C. Most typically, the catalyst is Pd(OH)₂/C. Typically, the hydrogen gas is provided at a pressure of 80-120 Psi, typically about 100 Psi. The catalyst and hydrogen gas may be used in the presence of an acid such as sulfuric acid or a sulfonic acid such as methanesulfonic acid or p-toluenesulfonic acid (PTSA). Most typically, Pd(OH)₂/C and hydrogen gas are used in the presence of methanesulfonic acid.

In one embodiment, the reduction of step (d) is carried out in the presence of a solvent. Typically, the solvent is a polar solvent such as methanol, ethanol, ethyl acetate, isopropanol, n-butanol, THF, water, acetic acid or a mixture thereof. Typically, the solvent is a polar protic solvent. More typically the solvent is an alcohol such as methanol, ethanol, isopropanol or n-butanol. Most typically, the solvent is methanol.

In one embodiment, the reduction of step (d) is carried out at a temperature in the range from 10 to 80° C. Typically, the reduction of step (d) is carried out at a temperature in the range from 20 to 60° C.

Typically, where the process of the invention comprises step (d), the 1,2,3,5,6,7-hexahydro-s-indacen-4-amine (D) is obtained in non-salt form.

In an exemplary embodiment, where a process of the invention comprises the use of 4-(phenoxycarbonylamino)-1,2,3,5,6,7-hexahydro-s-indacene (B′), the 4-(phenoxycarbonylamino)-1,2,3,5,6,7-hexahydro-s-indacene (B′) is prepared by a process comprising the steps of:
(a) contacting 2,3-dihydro-1H-indene (L) with 3-chloropropionyl chloride (M′) in the presence of a Lewis acid to obtain 3-chloro-1-(2,3-dihydro-1H-inden-5-yl)propan-1-one (N′):

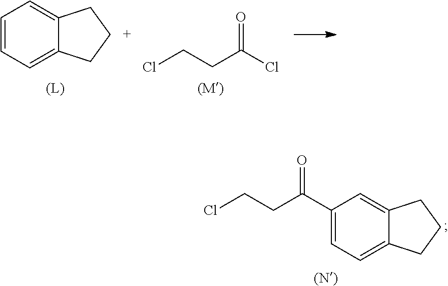

(b) contacting 3-chloro-1-(2,3-dihydro-1H-inden-5-yl)propan-1-one (N′) with an acid to obtain 1,2,3,5,6,7-hexahydro-s-indacen-1-one (P):

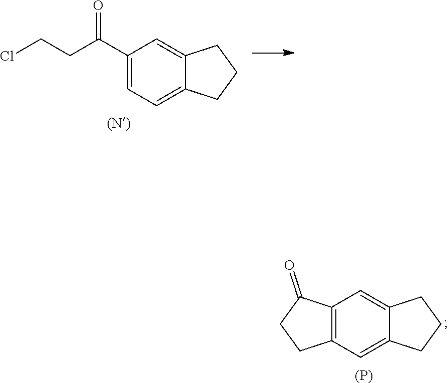

(c) converting 1,2,3,5,6,7-hexahydro-s-indacen-1-one (P) to 8-nitro-1,2,3,5,6,7-hexahydro-s-indacen-1-one (Qa) and/or 4-nitro-1,2,3,5,6,7-hexahydro-s-indacen-1-one (Qb) by treatment with sulfuric acid and nitric acid:

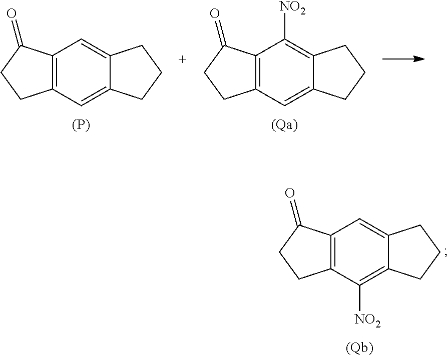

(d) reducing 8-nitro-1,2,3,5,6,7-hexahydro-s-indacen-1-one (Qa) and/or 4-nitro-1,2,3,5,6,7-hexahydro-s-indacen-1-one (Qb) to obtain 1,2,3,5,6,7-hexahydro-s-indacen-4-amine (D):

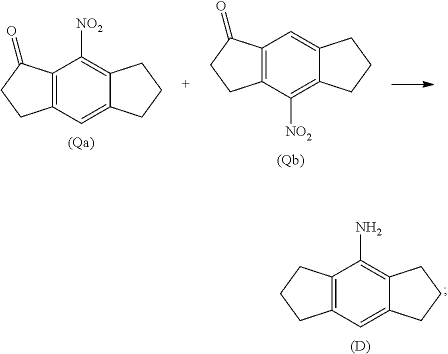

and
(e) converting the 1,2,3,5,6,7-hexahydro-s-indacen-4-amine (D) to the 4-(phenoxycarbonylamino)-1,2,3,5,6,7-hexahydro-s-indacene (B′) by contacting 1,2,3,5,6,7-hexahydro-s-indacen-4-amine with PhOC(O)L:

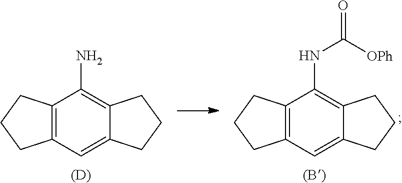

wherein L is selected from Cl and Br.

A fourth aspect of the invention provides an N-protected-4-hydroxy piperidine (G) or a salt thereof:

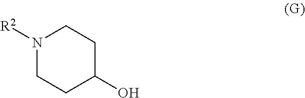

wherein R² is a nitrogen protecting group.

In the fourth aspect of the invention, R² may be as defined in accordance with any embodiment of any of the first to the third aspects of the invention.

A particular embodiment of the fourth aspect of the invention provides N-carboxybenzyl-4-hydroxy piperidine (G′) or a salt thereof:

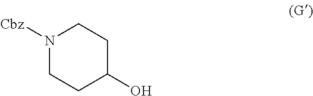 [SMILES: O=C(OCc1ccccc1)N1CCC(O)CC1]

The N-protected-4-hydroxy piperidine (G) or the salt thereof, or the N-carboxybenzyl-4-hydroxy piperidine (G′) or the salt thereof, may be prepared by or preparable by a process of step (i) of any of the first, second or third aspects of the invention. Typically, the N-protected-4-hydroxy piperidine (G) or the salt thereof, or the N-carboxybenzyl-4-hydroxy piperidine (G′) or the salt thereof, is prepared by or preparable by a process of step (i) of the third aspect of the invention.

Typically the N-protected-4-hydroxy piperidine (G) or the N-carboxybenzyl-4-hydroxy piperidine (G′) of the fourth aspect of the invention is in non-salt form.

A fifth aspect of the invention provides an N-protected-4-derivatised piperidine (H) or a salt thereof:

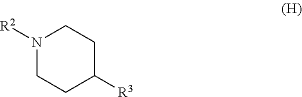

wherein R² is a nitrogen protecting group and R³ is a leaving group.

In the fifth aspect of the invention, R² and R³ may be as defined in accordance with any embodiment of any of the first to the third aspects of the invention.

A particular embodiment of the fifth aspect of the invention provides benzyl 4-((methylsulfonyl)oxy)piperidine-1-carboxylate (H′) or a salt thereof:

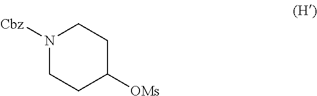 [SMILES: CS(=O)(=O)OC1CCN(C(=O)OCc2ccccc2)CC1]

The N-protected-4-derivatised piperidine (H) or the salt thereof, or the benzyl 4-((methylsulfonyl)oxy)piperidine-1-carboxylate (H′) or the salt thereof, may be prepared by or preparable by a process of step (ii) of any of the first, second or third aspects of the invention. Typically, the N-protected-4-derivatised piperidine (H) or the salt thereof, or the benzyl 4-((methylsulfonyl)oxy)piperidine-1-carboxylate (H′) or the salt thereof, is prepared by or preparable by a process of step (ii) of the third aspect of the invention.

Typically the N-protected-4-derivatised piperidine (H) or the benzyl 4-((methylsulfonyl)oxy)piperidine-1-carboxylate (H′) of the fifth aspect of the invention is in non-salt form.

A sixth aspect of the invention provides a thiourea adduct (I) or a salt thereof:

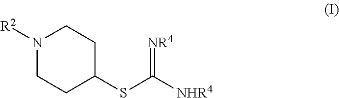

wherein:
R² is a nitrogen protecting group; and
each R⁴ is independently selected from hydrogen or a C₁-C₂₀ hydrocarbyl group, wherein each C₁-C₂₀ hydrocarbyl group may be straight-chained or branched, or be or include one or more cyclic groups, wherein each C₁-C₂₀ hydrocarbyl group may optionally be substituted with one or more oxo (═O) and/or one or more halo groups, and wherein each C₁-C₂₀ hydrocarbyl group may optionally include one or more heteroatoms independently selected from N, O and S in its carbon skeleton, or wherein any two or more R⁴ may, together with the atom or atoms to which they are attached, form a 3- to 16-membered heterocyclic group, wherein the heterocyclic group may be monocyclic, bicyclic or tricyclic, and wherein the heterocyclic group may optionally be substituted with one or more halo groups and/or one or more groups R⁴⁰, wherein each R⁴⁰ is independently selected from a —CN, —OH, —NH₂, oxo (═O), ═NH or C₁-C₆ hydrocarbyl group, wherein each C₁-C₆ hydrocarbyl group may be straight-chained or branched, or be or include one or more cyclic groups, wherein each C₁-C₆ hydrocarbyl group may optionally be substituted with one or more halo groups, and wherein each C₁-C₆ hydrocarbyl group may optionally include one or more heteroatoms independently selected from N, O and S in its carbon skeleton.

In the sixth aspect of the invention, R² and each R⁴ may be as defined in accordance with any embodiment of any of the first to the third aspects of the invention.

A particular embodiment of the sixth aspect of the invention provides benzyl 4-(carbamimidoylthio)-piperidine-1-carboxylate (I′) or a salt thereof:

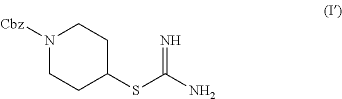 [SMILES: N=C(N)SC1CCN(C(=O)OCc2ccccc2)CC1]

The thiourea adduct (I) or the salt thereof, or the benzyl 4-(carbamimidoylthio)-piperidine-1-carboxylate (I′) or the salt thereof, may be prepared by or preparable by a process of step (iii) of any of the first, second or third aspects of the invention. Typically, thiourea adduct (I) or the salt thereof, or the benzyl 4-(carbamimidoylthio)-piperidine-1-carboxylate (I′) or the salt thereof, is prepared by or preparable by the process of the first aspect of the invention.

Typically, the thiourea adduct (I) or the benzyl 4-(carbamimidoylthio)-piperidine-1-carboxylate (I′) is in salt form. More typically, there is provided a sulfonic acid addition salt of the thiourea adduct (I) or the benzyl 4-(carbamimidoylthio)-piperidine-1-carboxylate (I′). Most typically, the sixth aspect of the invention provides the methanesulfonic acid salt of the thiourea adduct (I) or the benzyl 4-(carbamimidoylthio)-piperidine-1-carboxylate (I′).

Typically, the thiourea adduct (I) or the salt thereof, or the benzyl 4-(carbamimidoylthio)-piperidine-1-carboxylate (I′) or the salt thereof is in a solid form, most typically a crystalline solid form.

In one embodiment of the sixth aspect of the invention, the thiourea adduct (I) or the salt thereof has a HPLC purity of ≥90%. More typically, the thiourea adduct (I) or the salt thereof has a HPLC purity of ≥95%. More typically still, the thiourea adduct (I) or the salt thereof has a HPLC purity of ≥99%.

In another embodiment of the sixth aspect of the invention, the benzyl 4-(carbamimidoylthio)-piperidine-1-carboxylate (I′) or the salt thereof has a HPLC purity of 90%. More typically, the benzyl 4-(carbamimidoylthio)-piperidine-1-carboxylate (I′) or the salt thereof has a HPLC purity of ≥95%. More typically still, the benzyl 4-(carbamimidoylthio)-piperidine-1-carboxylate (I′) or the salt thereof has a HPLC purity of ≥99%.

A seventh aspect of the invention provides an N-protected-4-(halosulfonyl)-piperidine (J) or a salt thereof:

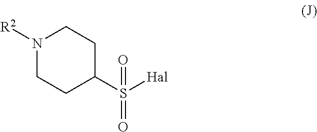

wherein R² is a nitrogen protecting group and Hal is Cl or Br.

In the seventh aspect of the invention, R² and Hal may be as defined in accordance with any embodiment of any of the first to the third aspects of the invention.

A particular embodiment of the seventh aspect of the invention provides benzyl 4-(chlorosulfonyl)-1-piperidinecarboxylate (J′) or a salt thereof:

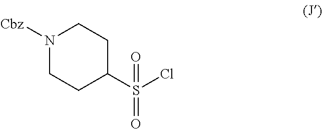 [SMILES: O=C(OCc1ccccc1)N1CCC(S(=O)(=O)Cl)CC1]

The N-protected-4-(halosulfonyl)-piperidine (J) or the salt thereof, or the benzyl 4-(chlorosulfonyl)-1-piperidinecarboxylate (J′) or the salt thereof, may be prepared by or preparable by a process of step (iv) of the second or third aspect of the invention. Typically, the N-protected-4-(halosulfonyl)-piperidine (J) or the salt thereof, or the benzyl 4-(chlorosulfonyl)-1-piperidinecarboxylate (J′) or the salt thereof, is prepared by or preparable by a process of the second aspect of the invention.

Typically the N-protected-4-(halosulfonyl)-piperidine (J) or the benzyl 4-(chloro-sulfonyl)-1-piperidinecarboxylate (J′) of the seventh aspect of the invention is in non-salt form.

Typically, the N-protected-4-(halosulfonyl)-piperidine (J) or the salt thereof, or the benzyl 4-(chlorosulfonyl)-1-piperidinecarboxylate (J′) or the salt thereof is in a solid form, most typically a crystalline solid form.

In one embodiment of the seventh aspect of the invention, the N-protected-4-(halosulfonyl)-piperidine (J) or the salt thereof has a HPLC purity of ≥90%. More typically, the N-protected-4-(halosulfonyl)-piperidine (J) or the salt thereof has a HPLC purity of ≥95%. More typically still, the N-protected-4-(halosulfonyl)-piperidine (J) or the salt thereof has a HPLC purity of ≥99%.

In one embodiment of the seventh aspect of the invention, the benzyl 4-(chlorosulfonyl)-1-piperidinecarboxylate (J′) or the salt thereof has a HPLC purity of 90%. More typically, the benzyl 4-(chlorosulfonyl)-1-piperidinecarboxylate (J′) or the salt thereof has a HPLC purity of ≥95%. More typically still, the benzyl 4-(chlorosulfonyl)-1-piperidinecarboxylate (J′) or the salt thereof has a HPLC purity of 99%.

An eighth aspect of the invention provides an N-protected-4-piperidinesulfonamide (K)

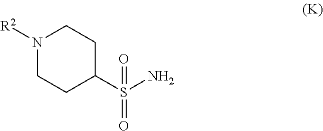

wherein R² is a nitrogen protecting group.

In the eighth aspect of the invention, R² may be as defined in accordance with any embodiment of any of the first to the third aspects of the invention.

A particular embodiment of the eighth aspect of the invention provides 1-(benzyloxycarbonyl)-4-piperidinesulfonamide (K′) or a salt thereof:

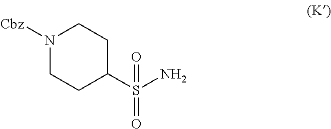 [SMILES: NS(=O)(=O)C1CCN(C(=O)OCc2ccccc2)CC1]

The N-protected-4-piperidinesulfonamide (K) or the salt thereof, or the 1-(benzyloxycarbonyl)-4-piperidinesulfonamide (K′) or the salt thereof, may be prepared by or preparable by a process of step (v) of the second or third aspect of the invention. Typically, the N-protected-4-piperidinesulfonamide (K) or the salt thereof, or the 1-(benzyloxycarbonyl)-4-piperidinesulfonamide (K′) or the salt thereof, is prepared by or preparable by a process of step (v) of the third aspect of the invention.

Typically the N-protected-4-piperidinesulfonamide (K) or the 1-(benzyloxycarbonyl)-4-piperidinesulfonamide (K′) of the eighth aspect of the invention is in non-salt form.

Typically, the N-protected-4-piperidinesulfonamide (K) or the salt thereof, or the 1-(benzyloxycarbonyl)-4-piperidinesulfonamide (K′) or the salt thereof, is in a solid form, most typically a crystalline solid form.

In one embodiment of the eighth aspect of the invention, the N-protected-4-piperidinesulfonamide (K) or the salt thereof has a HPLC purity of ≥96.2%. More typically, the N-protected-4-piperidinesulfonamide (K) or the salt thereof has a HPLC purity of ≥98%. More typically still, the N-protected-4-piperidinesulfonamide (K) or the salt thereof has a HPLC purity of ≥99.5%.

In another embodiment of the eighth aspect of the invention, the 1-(benzyloxycarbonyl)-4-piperidinesulfonamide (K′) or the salt thereof has a HPLC purity of 96.2%. More typically, the 1-(benzyloxycarbonyl)-4-piperidinesulfonamide (K′) or the salt thereof has a HPLC purity of ≥98%. More typically still, the 1-(benzyloxycarbonyl)-4-piperidinesulfonamide (K′) or the salt thereof has a HPLC purity of 99.5%.

A ninth aspect of the invention provides 1-ethyl-4-piperadinesulfonamide (A) or a salt thereof:

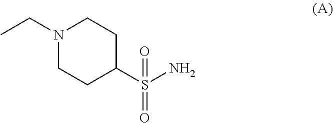

The 1-ethyl-4-piperadinesulfonamide (A) or the salt thereof may be prepared by or preparable by a process of step (vi) of the second or third aspect of the invention. Typically, the 1-ethyl-4-piperadinesulfonamide (A) or the salt thereof is prepared by or preparable by a process of step (vi) of the third aspect of the invention

Typically the 1-ethyl-4-piperadinesulfonamide (A) of the eleventh aspect of the invention is in non-salt form.

A tenth aspect of the invention provides 1-ethyl-N-((1,2,3,5,6,7-hexahydro-s-indacen-4-yl)carbamoyl)piperidine-4-sulfonamide or a salt thereof, prepared by or preparable by a process of any of the first to third aspects of this invention.

In one embodiment, the tenth aspect of the invention provides an alkali metal or an alkali earth metal salt of 1-ethyl-N-((1,2,3,5,6,7-hexahydro-s-indacen-4-yl)carbamoyl)-piperidine-4-sulfonamide. Typically, the tenth aspect of the invention provides a potassium salt of 1-ethyl-N-((1,2,3,5,6,7-hexahydro-s-indacen-4-yl)carbamoyl)-piperidine-4-sulfonamide. Most typically, the tenth aspect of the invention provides a mono-potassium salt of 1-ethyl-N-((1,2,3,5,6,7-hexahydro-s-indacen-4-yl)carbamoyl)-piperidine-4-sulfonamide.

In one embodiment of the tenth aspect of the invention, the 1-ethyl-N-((1,2,3,5,6,7-hexahydro-s-indacen-4-yl)carbamoyl)piperidine-4-sulfonamide or the salt thereof has a purity as measured by ¹H NMR of 97.0%. More typically, the 1-ethyl-N-((1,2,3,5,6,7-hexahydro-s-indacen-4-yl)carbamoyl)piperidine-4-sulfonamide or the salt thereof has a purity as measured by ¹H NMR of 98.0%, or 99.0%, or 99.5%.

In another embodiment of the tenth aspect of the invention, the 1-ethyl-N-((1,2,3,5,6,7-hexahydro-s-indacen-4-yl)carbamoyl)piperidine-4-sulfonamide or the salt thereof has a HPLC purity of ≥95.0%. More typically, the 1-ethyl-N-((1,2,3,5,6,7-hexahydro-s-indacen-4-yl)carbamoyl)piperidine-4-sulfonamide or the salt thereof has a HPLC purity of ≥98.0%, or 99.0%, or 99.5%, or 99.8%, or 99.9%.

The compounds used in and provided by the processes of the present invention can be used in and provided, as appropriate, in their free base form or their acid addition salt form. For the purposes of this invention, a “salt” of a compound of the invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound used in or provided by a process of the invention includes a quaternary ammonium group, typically the compound is used or provided in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds used in and provided by the processes of the present invention can also be used in and provided, as appropriate, in their free acid form and their salt form. For the purposes of this invention, a “salt” of a compound of the present invention includes one formed between a protic acid functionality (such as a carboxylic acid group or a urea group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in connection with the present invention, since they have potential to serve as intermediates in the purification or preparation of other, for example, pharmaceutically acceptable salts, or are useful for identification, characterisation or purification of the free acid or base.

The compounds and/or salts used in and provided by the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

The compounds, salts and solvates used in and provided by the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

Unless stated otherwise, the compounds, salts and solvates used in and provided by the present invention may be in any polymorphic or amorphous form.

An eleventh aspect of the present invention provides a pharmaceutical composition comprising the 1-ethyl-N-((1,2,3,5,6,7-hexahydro-s-indacen-4-yl)carbamoyl)-piperidine-4-sulfonamide or the salt thereof of the tenth aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013. Pharmaceutically acceptable excipients including adjuvants, diluents or carriers that may be used in the pharmaceutical compositions of the invention, are those conventionally employed in the field of pharmaceutical formulation.

A twelfth aspect of the present invention provides the 1-ethyl-N-((1,2,3,5,6,7-hexahydro-s-indacen-4-yl)carbamoyl)piperidine-4-sulfonamide or the salt thereof of the tenth aspect of the invention, or the pharmaceutical composition of the eleventh aspect of the invention, for use in medicine, and/or for use in the treatment or prevention of a disease, disorder or condition.

Most especially, where 1-ethyl-N-((1,2,3,5,6,7-hexahydro-s-indacen-4-yl)-carbamoyl)piperidine-4-sulfonamide is used in the treatment or prevention of a disease, disorder and condition, the 1-ethyl-N-((1,2,3,5,6,7-hexahydro-s-indacen-4-yl)-carbamoyl)piperidine-4-sulfonamide acts as an NLRP3 inhibitor.

In one embodiment, the disease, disorder or condition to be treated or prevented is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xy) pain; and
(xyi) any disease where an individual has been determined to carry a germline or somatic non-silent mutation in NLRP3.

Typically, the treatment or prevention of the disease, disorder or condition comprises the administration of the 1-ethyl-N-((1,2,3,5,6,7-hexahydro-s-indacen-4-yl)carbamoyl)-piperidine-4-sulfonamide or the salt thereof of the tenth aspect of the invention, or the pharmaceutical composition of the eleventh aspect of the invention, to a subject.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal or topical (including transdermal, buccal, mucosal and sublingual) administration.

Typically, the mode of administration selected is that most appropriate to the disorder, disease or condition to be treated or prevented.

A thirteenth aspect of the invention provides a method of inhibiting NLRP3, the method comprising the use of the 1-ethyl-N-((1,2,3,5,6,7-hexahydro-s-indacen-4-yl)-carbamoyl)piperidine-4-sulfonamide or the salt thereof of the tenth aspect of the invention, or the pharmaceutical composition of the eleventh aspect of the invention, to inhibit NLRP3.

For the avoidance of doubt, insofar as is practicable any embodiment of a given aspect of the present invention may occur in combination with any other embodiment of the same aspect of the present invention. In addition, insofar as is practicable it is to be understood that any preferred, typical, optional or exemplary embodiment of any aspect of the present invention should also be considered as a preferred, typical, optional or exemplary embodiment of any other aspect of the present invention.

### EXAMPLES

All solvents, reagents and compounds were purchased and used without further purification unless stated otherwise.

### Abbreviations

Aq: aqueous
Cbz: carboxybenzyl/benzyloxycarbonyl
GC: gas chromatography
HPLC: high performance liquid chromatography
THF: tetrahydrofuran
RBF: round bottom flask
Eq: equivalent(s)
min: minute(s)
h: hour(s)
MTBE: methyl tertiary butyl ether
Ms: mesyl
DCM: dichloromethane
DMFL dimethylformamide
TEA: trimethylamine
IPA: isopropyl alcohol
HDPE: high density polyethylene
NMT: No more than
NCS: N-chlorosuccinimide
V, vol: volumes
AKX reagent: AQUAMICRON® AKX

% a/a:(area under peak of compound (a)/combined area under peaks of compound (a) and all other components)×100

As used herein, unless stated otherwise all references to a pressure in bar refer to the absolute pressure.

### Experimental Methods

### NMR Methods:

NMR spectra were obtained on Bruker AV 400 MHz spectrometer (model: Advance IIID) operated at room temperature (25° C.).

### GC Methods:

GC analysis was conducted on one of the following machines: Agilent 7890, 6890, or Agilent 6890N with ALS injector.

### HPLC Methods:

HPLC in reaction scheme 1, steps (i)-(vi) was run on Agilent 1260 Infinity II HPLC with UV detector using 0.05% TFA in water as mobile phase-A and 0.05% TFA in acetonitrile as mobile phase-B.

HPLC in reaction scheme 2, steps (a)-(d) was run on Waters Alliance e2695 HPLC with PDA detector using 10 Mm ammonium bicarbonate in water as mobile phase-A and acetonitrile as mobile phase-B.

HPLC in reaction scheme 3 was run using ammonium acetate in water: MeCN (for both mobile phases) on Agilent 1100, 1200, or 1260.

As used herein, unless stated otherwise all references to HPLC purity are measured as the % a/a.

### KF Methods:

Coulometric KF (Karl Fischer) titration was run using AKX reagent on Mitsubishi CA-20 or Predicta OM1000.

### SYNTHESIS EXAMPLES

### 1-ethyl-4-piperidinesulfonamide (7)

1-ethyl-4-piperidinesulfonamide (7) was prepared according to the reaction sequence illustrated in reaction scheme 1:

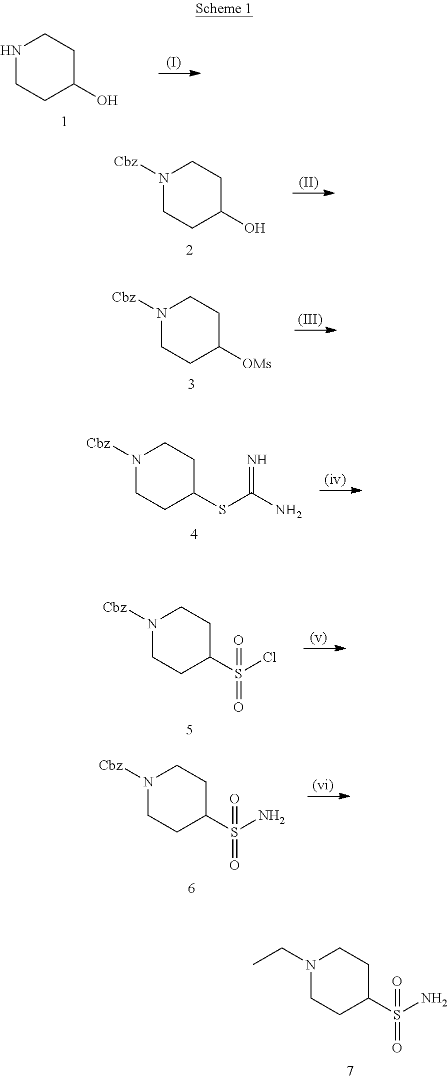

### Reaction Scheme 1—Step (i)

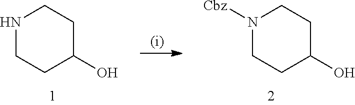

4-hydroxy piperidine (1) (20.0 g, 198 mmol, 1.0 eq.) was charged together with toluene (80 ml, 4 V), water (30 ml, 1.5 V) and 30% NaOH aq. (40.2 g, 297 mmol, 1.5 eq.) to a 250 ml glass reactor and the mixture was cooled to 0-5° C. A solution of CbzCl (33.7 g, 198 mmol, 1.0 eq.) in toluene (30 ml, 1.5 V) was dosed to the vigorously stirred biphasic mixture at 0-10° C. The reaction mixture was warmed to 20-25° C. and the aq. layer was drained. The organic layer was washed twice with water (20 ml, 1 V) and subsequently dried by azeotropic distillation to afford a solution of benzyl 4-hydroxy-1-piperidinecarboxylate (2) in toluene. The obtained solution was used directly in the next step without further purification. Alternatively, the solvent may be removed and the residue redissolved in toluene to provide the reaction mixture for the next step.

### Reaction Scheme 1—Step (ii)

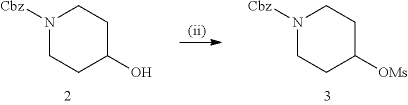 [SMILES: O=C(OCc1ccccc1)N1CCC(O)CC1 ; CS(=O)(=O)OC1CCN(C(=O)OCc2ccccc2)CC1]

Benzyl 4-hydroxy-1-piperidinecarboxylate (2) (200.0 g, 850 mmol, 1.0 eq.), MsCl (102.2 g, 893 mol, 1.05 eq.) and toluene (600 ml, 3 V) were charged to a 1.0 L glass reactor. The thin slurry was cooled to 0-5° C. Subsequently, triethylamine (94.6 g, 130 ml, 935 mmol, 1.1 eq.) was added dropwise while keeping the temperature at 0-5° C. The reaction is strongly exothermic and proceeds completely with controlled addition so as to limit the rate of reaction. The jellylike slurry was aged for at least 10 min before being analysed by HPLC (starting material<0.5% a/a). Water (200 ml, 2 V) was added to the reaction mixture and the temperature was adjusted to 10-25° C. The aq. layer was drained, and the organic layer was washed with water (200 ml, 2 V). From the remaining organic layer, 400 ml (2 V) was distilled under reduced pressure to obtain a solution of benzyl 4-((methylsulfonyl)oxy)piperidine-1-carboxylate (3) in toluene. The obtained solution was directly used in the next step without further purification.

Reaction Scheme 1—Step (iii)

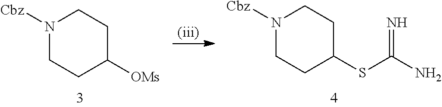 [SMILES: CS(=O)(=O)OC1CCN(C(=O)OCc2ccccc2)CC1 ; "(iii)" ; N=C(N)SC1CCN(C(=O)OCc2ccccc2)CC1]

To the solution of the crude benzyl 4-((methylsulfonyl)oxy)piperidine-1-carboxylate (3) in toluene from step (ii) was added n-butanol (600 ml, 3 V) and thiourea (71.2 g, 935 mmol, 1.1 eq.). Under reduced pressure, 200 ml (1 V) was distilled off and replaced by n-butanol (200 ml, 1 V). Then, the temperature was adjusted to 95-100° C. After stirring for 1 h at this temperature, the reaction mixture was seeded with thiourea adduct (4) (prepared from an earlier batch without seeding) and stirred for another 4-5 h. Afterwards, the slurry was cooled to 20-25° C. over at least 2 h and aged for at least 1 h. The solid was filtered off and the wet cake was washed with IPA (200 ml, 1 V). The solid was dried in the vacuum cabinet at 50° C. to give the methanesulfonic acid salt of benzyl 4-(carbamimidoylthio)-piperidine-1-carboxylate (4) (175.2 g) as a colourless solid.

Yield: 53% over three steps

HPLC purity: 99.4%

¹H NMR (400 MHz, DMSO-d₆): δ 9.17 (s, 4H), 7.67-7.19 (m, 5H), 5.08 (s, 2H), 4.12-3.75 (m, 3H), 2.41 (s, 3H), 1.99 (dd, J=13.2, 3.8 Hz, 2H), 1.52 (dtd, J=13.0, 10.9, 4.1 Hz, 2H).

¹³C NMR (101 MHz, DMSO): δ 168.68, 154-72, 137-30, 128.89, 128-34, 128.06, 66.79, 43.35, 41.39, 40.24, 31.76.

### Reaction Scheme 1—Step (iv)

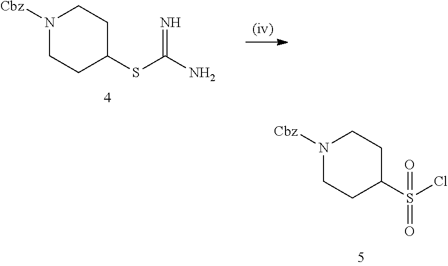 [SMILES: N=C(N)SC1CCN(C(=O)OCc2ccccc2)CC1 ; O=C(OCc1ccccc1)N1CCC(S(=O)(=O)Cl)CC1]

The methanesulfonic acid salt of benzyl 4-(carbamimidoylthio)-piperidine-1-carboxylate (4) (100.0 g, 256.7 mmol, 1.0 eq.) was charged to a mixture of acetic acid (200 ml, 2 V) and 10% w/w hydrochloric acid (100 ml, 1.0 V) and the temperature was adjusted to 25-30° C. Most of the starting material dissolved endothermically and a thin suspension was obtained. Then, NCS (101.1 g, 757.4 mmol, 2.95 eq.) was added in at least 10 portions over at least 1 h at 25-30° C. After about 25% of the NCS addition, a clear solution was obtained. After 50% addition, the mixture was seeded with sulfonyl chloride (5) (prepared from an earlier batch without seeding). After the crystallization had started, dosing of NCS was pursued. The oxidation with NCS is exothermic and proceeds well with controlled addition so as to limit the rate of reaction. After the addition, the reaction mixture was aged for at least 30 min at 20-25° C. before the conversion was checked by HPLC. Additional NCS was added based on the HPLC result (area % starting material=mol % NCS to be added additionally) and the conversion was checked again after 30 min (target: starting material<1% a/a). Residual NCS was quenched with 20% w/w Na₂SO₃ until a KI/starch test was negative. Then, water (100 ml, 1 V) was added over at least 15 min and the temperature was adjusted to 18-22° C. After stirring for at least 1 h, the product was isolated by filtration and the filter cake was washed with AcOH/water 1:1 (100 ml, 1V) and water (100 ml, 1 V). The wet product was dried in the vacuum cabinet at 50° C. to give 76.0 g benzyl 4-(chlorosulfonyl)-1-piperidinecarboxylate (5) as a colourless solid.

Yield: 93.2%

HPLC purity: 99.10%

¹H NMR (400 MHz, CDCl₃): δ 7.49-7.32 (m, 5H), 5.17 (s, 2H), 4.43 (s, 2H), 3.69 (tt, J==11.8, 3.8 Hz, 1H), 2.92 (s, 2H), 2.38 (d, J=12.9 Hz, 2H), 1.97 (qd, J=12.1, 11.5, 4.0 Hz, 2H).

¹³C NMR (101 MHz, CDCl₃): δ 154.81, 136.22, 128.61, 128-30, 128.10, 72.16, 67.68, 42.54, 26.59.

### Reaction Scheme 1—Step (v)

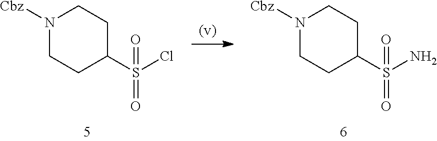 [SMILES: O=C(OCc1ccccc1)N1CCC(S(=O)(=O)Cl)CC1 ; NS(=O)(=O)C1CCN(C(=O)OCc2ccccc2)CC1]

THF (300 ml, 6 V) was charged to a dry 500 ml glass reactor and the temperature was adjusted to −5 to 0° C. The atmosphere was changed to ammonia and the solution stirred at 1000 rpm until no further ammonia uptake was observed. At −5 to 5° C., benzyl 4-(chlorosulfonyl)-1-piperidinecarboxylate (5) (50.0, 157.3 mmol, 1.0 eq.) was added in at least 5 portions over at least 1 h while continuing stirring at 1000 rpm under NH₃ atmosphere. The addition of the starting material proceeded along with a slight outgassing of ammonia, caused by the temperature increase. The light slurry was aged for at least 15 min and the conversion was checked by HPLC (sulfonyl chloride (5)<0.5% a/a). After distillation of 150-200 ml at normal pressure, the residue was diluted with AcOiPr (100 ml) and water (so ml). The aq. layer was drained at 50-70° C.

Subsequently, 200 ml was distilled off at normal pressure while keeping the volume constant by feeding AcOiPr (200 ml). Then, water (50 ml) was added, and the temperature was adjusted to 60° C. The biphasic mixture was seeded with the sulfonamide (6) (prepared from an earlier batch without seeding) and the slurry cooled to 20° C. over at least 1 h. The product was isolated by filtration and the filter cake was washed with water (25 ml) and AcOiPr (25 ml). The wet product (51 g) was dried in the vacuum cabinet at 50° C. to give 1-(benzyloxycarbonyl)-4-piperidinesulfonamide (6) (42.7 g) as a colourless solid.

Yield: 91.0%

HPLC purity: 99.6%

¹H NMR: (DMSO 400 MHz): δ 1.41-1.51 (m, 2H), δ 1.99-2.01 (m, 2H), δ 2.50-286 (m, 2H), δ 3.022-3.05 (m, 1H) S 4.08-4.11 (m, 2H), δ 5.75 (s, 2H) δ 6.78 (s, 2H), δ 7.40-7.30 (m, 5H)

### Reaction Scheme 1—Step (vi)

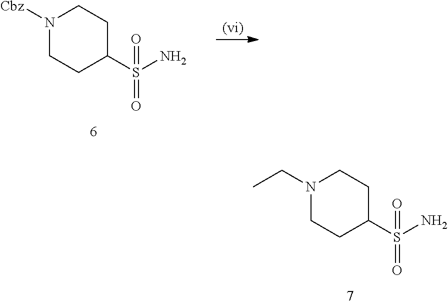 [SMILES: NS(=O)(=O)C1CCN(C(=O)OCc2ccccc2)CC1 ; CCN1CCC(S(N)(=O)=O)CC1 ; "7"]

The combined Cbz-deprotection/ethylation was performed using 10% Pd/C as the catalyst and water saturated 1-butanol as the solvent. Specifically, 1-(benzyloxycarbonyl)-4-piperidinesulfonamide (6) (2.0 g), water (4.0 ml), 1-butanol (16.0 ml), acetonitrile (0.6 ml) and 10% Pd/C (0.20 g) were placed in a Hastelloy autoclave and hydrogentated at 10-20 bar for 24 h. The catalyst was filtered off and the filtrate concentrated to dryness to leave 1-ethyl-4-piperidinesulfonamide (7) as colourless solid. After hydrogenation with 10-20 bar H₂ at 60° C. overnight, clean and quantitative conversion with no by-products was detected (¹H-NMR).

¹H NMR: (DMSO) 0.95 (t), 1.55 (dq), 1.80 (app t), 1.95 (app d), 2.30 (q), 2.75 (m), 2.90 (app d)

Reaction Scheme 1—Step (vi)—Alternate Procedure

1-(benzyloxycarbonyl)-4-piperidinesulfonamide (6) (21.85 Kg) was charged to a vessel which was then purged with nitrogen. Ethanol (85.2 Kg) and purified water (43.7 L) were charged to the vessel and the temperature was adjusted to 15 to 25° C. The vessel was vacuum/nitrogen purged three times at 15 to 25° C. and then charged with palladium hydroxide on carbon (20 wt %; 50% water) (0.66 Kg). The vessel was vacuum/nitrogen purged three times at 15 to 25° C. The vessel was vacuum/hydrogen purged three times at 15 to 25° C. and maintained under an atmosphere of hydrogen (ca. 3 bar). The reaction mixture was stirred until complete. Completion was measured by ¹H NMR analysis, pass criterion≤5.0 mol % 1-(benzyloxycarbonyl)-4-piperidinesulfonamide (6).

The vessel was vacuum/nitrogen purged three times at 15 to 25° C. and then charged with palladium hydroxide on carbon (20 wt %; 50% water) (1.09 Kg) as a slurry in water (21.85 Kg) and acetonitrile (9.2 Kg) at 15 to 25° C. The vessel was heated to 35 to 45° C. and vacuum/nitrogen purged three times at 15 to 25° C. The vessel was vacuum/hydrogen purged three times at 15 to 25° C. and maintained under an atmosphere of hydrogen (ca. 3 bar).

The reaction mixture was stirred at 15 to 25° C. until complete. At approximately 6 hours intervals the reaction vessel was purged with vacuum/hydrogen to remove ammonia. Completion was measured by ¹H NMR analysis, pass criterion≤5.0 mol % 4-piperidinesulfonamide.

Once the pass criterion by ¹H NMR analysis was met, the reaction mixture was stirred at 15 to 25° C. until complete by GC analysis. Pass criterion≤0.05% combined area of 4-piperidinesulfonamide plus intermediate at relative retention time: 0.939 intermediate.

Once the reaction was deemed complete by GC, the vessel was purged with nitrogen and the reaction mixture cooled to 15 to 25° C. and filtered through a 1 μm filter at 15 to 25° C. to remove the catalyst. The filter cake was twice washed with pre-mixed purified water and ethanol (13.1 Kg:10.9 Kg and 13.1 Kg:10.9 Kg) at 15 to 25° C.

The filtrate was charged with decolourising charcoal (activated) (4.37 Kg) and stirred at 15 to 25° C. for at least 60 minutes (target 60 to 120 minutes). The mixture was filtered through a 1 μm filter at 15 to 25° C. to remove the charcoal. The filter cake was washed twice with pre-mixed purified water and ethanol (13.1 Kg:10.9 Kg and 13.1 Kg:10.9 Kg) at to 25° C.

The filtrate was charged to a vessel and adjusted to 50 to 60° C., concentrated under reduced pressure at 50 to 60° C. to ca 110 L. n-Butanol (89.8 Kg) was charged at 50 to 60° C. and the mixture was concentrated under reduced pressure at 50 to 60° C. to ca 110 L. n-Butanol (86.9 Kg) was charged at 50 to 60° C. and the mixture was concentrated under reduced pressure at 50 to 60° C. to ca 110 L. n-Butanol (88.4 Kg) was charged at 50 to 60° C. and the mixture was concentrated under reduced pressure at 50 to 60° C. to ca 90 L. The supernatant of the concentrated mixture was analysed for water content by KF analysis, pass criterion≤0.5% w/w water.

The temperature was adjusted to 15 to 25° C. and ethyl acetate (98.6 Kg) was charged at to 25° C. The reaction mixture was cooled to −2 to +2° C. over at least 60 minutes (target 60 to 120 minutes). The mixture was stirred at −2 to 2° C. for at least 4 hours (target 4 to 6 hours). The solid was filtered on 20 μm filter cloth at −2 to 2° C. and washed twice with ethyl acetate, (38.1 Kg and 39.9 Kg) at −2 to 2° C.

The solid was dried at up to 60° C. under a flow of nitrogen until the n-butanol content was ≤0.5% w/w, ethanol content≤0.5% w/w, and ethyl acetate content was ≤0.5% w/w (measured by ¹H NMR spectroscopy). The dried weight of the solid 1-ethyl-4-piperidinesulfonamide (7) was measured and assayed using ¹H NMR spectroscopy.

Output: 10.98 Kg

Yield: 78%

¹H NMR: (DMSO) 0.95 (t), 1.55 (dq), 1.80 (app t), 1.95 (app d), 2.30 (q), 2.75 (m), 2.90 (app d)

### 4-(phenoxycarbonylamino)-1,2,3,5,6,7-hexahydro-s-indacene (13)

4-(phenoxycarbonylamino)-1,2,3,5,6,7-hexahydro-s-indacene (13) was prepared according to the reaction sequence illustrated in Reaction Scheme 2.

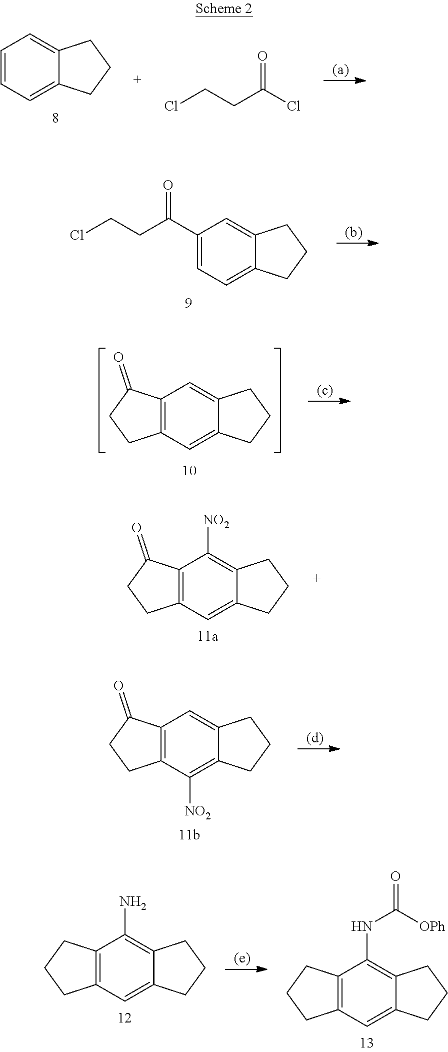

### Reaction Scheme 2—Step (a)

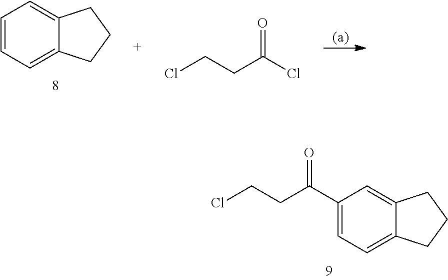 [SMILES: c1ccc2c(c1)CCC2 ; O=C(Cl)CCCl ; O=C(CCCl)c1ccc2c(c1)CCC2]

Reagents had methanol content of no more than 0.5% by GC.

DCM (385 L) and AlCl₃ (99.86 Kg) were charged at 25 to 30° C. under a nitrogen atmosphere into a 2.0 KL clean and dry glass-lined reactor. The reaction mixture was cooled to −10′ C. 3-chloropropanoyl chloride (90.99 Kg) was added slowly at −10 to -5° C. under a nitrogen atmosphere. The reaction mixture was maintained for 30 minutes at −10° C. under a nitrogen atmosphere. 2,3-dihydro-1H-indene (8) (77.00 Kg was then added slowly to the reaction mixture at −10 to −5° C. under nitrogen atmosphere. The reaction mixture was maintained for 2 hours at 10 to 15° C. The absence of 2,3-dihydro-1H-indene (8) was confirmed by HPLC (Limit: 5.0%).

After completion of the reaction, the reaction mixture was added slowly to a 6 N hydrochloric acid solution (prepared from water (308 L) and conc. hydrochloric acid (308 L)) at 0 to 10° C. DCM (231 L) was added and the reaction mixture temperature was raised to 30 to 35° C. The reaction mixture was stirred at 30 to 35° C. for 30 minutes and allowed to settle at 30 to 35° C. for 30 minutes. The layers were separated and the organic layer (OL-1) was kept aside. DCM (231 L) was charged to the aqueous layer at 25 to 30° C. The reaction mixture was stirred at 25 to 30° C. for 30 minutes and allowed to settle at 25 to 30° C. for 30 minutes. The layers were separated (aqueous layer (AL-1) and organic layer (OL-2)) and AL-1 was kept aside. OL-1 and OL-2 were combined at 25 to 30° C. Demineralised water (385 L) was added to the combined organic layers. The reaction mixture was stirred at 25 to 30° C. for 30 minutes and allowed to settle at 25 to 30° C. for 30 minutes. The layers were separated (aqueous layer (AL-2) and organic layer (OL-3)) and AL-2 was kept aside.

10% Saturated sodium bicarbonate solution (prepared from demineralised water (385 L) and sodium bicarbonate (38.5 Kg)) was charged to OL-3 at 25 to 30° C. The reaction mixture was stirred at 25 to 30° C. for 30 minutes and allowed to settle at 25 to 30° C. for 30 minutes. The layers were separated (aqueous layer (AL-3) and organic layer (OL-4)) and AL-3 was kept aside. OL-4 was dried over anhydrous Na₂SO₄ (38.5 Kg) and the anhydrous Na₂SO₄ was washed with DCM (150 L) at 25 to 30° C.

The solvent was distilled under vacuum at below 35 to 40° C. until 5% remained. n-hexane (308 L) was charged to the reaction mixture at 35 to 40° C. and the solvent was distilled completely at 35 to 40° C. until no condensate drops were formed. n-hexane (150 L) was charged to the reaction mixture at 35 to 40° C. and the reaction mixture was cooled to 5 to 10° C. and maintained at 5 to 10° C. for 30 minutes.

The solid product was filtered, washed with cooled hexane (77 L), and dried in a hot air oven at 40 to 45° C. for 6 hours to afford 120.5 Kg of 3-chloro-1-(2,3-dihydro-1H-inden-5-yl)propan-1-one (9)

Yield: 88.63%

HPLC purity: 99.3%

Moisture content: 0.09%

¹H NMR: (500 MHz, CDCl3): δ 7.81 (S, 1H), 7.76 (d, 1H), 7.31 (d, 1H), 3.93 (t, 2H), 3.45 (t, 2H), 2.97 (t, 4H), 2.15 (q, 2H)

### Reaction Scheme 2—Steps (b) and (c)

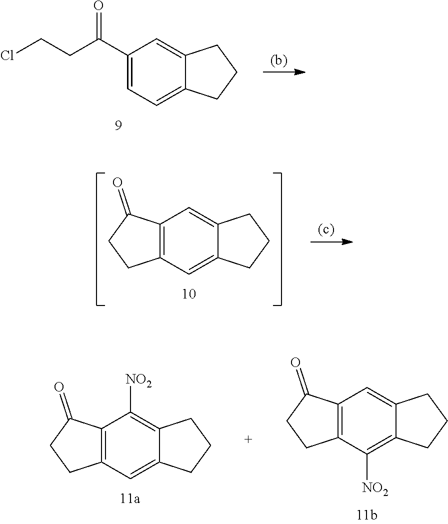

Sulfuric acid (300.0 L) was charged at 25 to 30° C. into a 2.0 KL clean and dry glass-lined reactor. 3-chloro-1-(2,3-dihydro-1H-inden-5-yl)propan-1-one (9) (60.0 Kg) was charged lot wise at 25 to 30° C. and the reaction mixture was maintained for 30 minutes at 25 to 30° C. The reaction mixture was slowly heated to 65 to 70° C. and maintained at 65 to 70° C. for 24 hours. The absence of 3-chloro-1-(2,3-dihydro-1H-inden-5-yl)-propan-1-one (9) was confirmed by HPLC (Limit: ≤1.0%).

Then the reaction mixture was cooled to 0 to 5° C. A nitration mixture*¹ was added slowly at 0 to 5° C. and the reaction mixture was maintained at 0 to 5° C. for 1 hour. The absence of 1,2,3,5,6,7-hexahydro-s-indacen-1-one (10) was confirmed by HPLC (Limit: 1.0%). The reaction mixture was maintained at 0 to 5° C. *1: To prepare the nitration mixture, sulfuric acid (27.0 L) was charged at 25 to 30° C. into a 160 L clean and dry glass-lined reactor. The reaction mixture was cooled to 0 to 5° C. Nitric acid (27.0 L) at 0 to 5° C. was added slowly and the reaction mixture was maintained for 30 minutes at 0 to 5° C. to afford the nitration mixture.

Demineralised water (900.0 L) was charged at 25 to 30° C. into a 2.0 KL clean and dry glass-lined reactor. The water was cooled to 0 to 5° C. The reaction mixture was added slowly added to the reactor at 0 to 5° C. Toluene (480.0 L) was added and the temperature was raised to 30 to 35° C. The reaction mixture was maintained at 30 to 35° C. for 30 minutes and allowed to settle at 30 to 35° C. for 30 minutes. The reaction mixture was filtered through a Celite® bed (prepared with Celite® (6.0 Kg) and toluene (30.0 L)). The Celite® bed was washed with toluene (60.0 L). The solid was filtered and sucked dry for 30 min.

The reaction mixture was charged to a 2.0 KL clean and dry glass-lined reactor. The reaction mixture was allowed to settle at 30 to 35° C. for 30 minutes. The layers were separated (aqueous layer (AL-1) and organic layer (OL-1)) and OL-1 was kept aside. Toluene (60.0 L) was charged to AL-1. The reaction mixture was stirred at 35 to 40° C. for 30 minutes and allowed to settle at 35 to 40° C. for 30 minutes. The layers were separated (aqueous layer (AL-2) and organic layer (OL-2)) and OL-2 was kept aside. OL-1 and OL-2 were combined to form OL-3.

A 5% saturated sodium bicarbonate solution (prepared from demineralised water (300.0 L) and sodium bicarbonate (15.0 Kg)) was slowly charged to OL-3 at 30 to 35° C. The reaction mixture was stirred at 35 to 40° C. for 30 minutes and allowed to settle at 35 to 40° C. for 30 minutes. The reaction mixture was filtered through a Celite® bed (prepared with Celite® (6.0 Kg) and demineralised water (60.0 L)). The Celite® bed was washed with toluene (60.0 L).

The reaction mixture was charged to a 3.0 KL clean and dry glass-lined reactor. The reaction mixture was allowed to settle at 30 to 35° C. for 30 minutes. The layers were separated (aqueous layer (AL-3) and organic layer (OL-4)) and OL-4 was kept aside.

Toluene (60.0 L) was charged to AL-3. The layers were separated (aqueous layer (AL-4) and organic layer (OL-5)) and OL-5 was kept aside. OL-4 and OL-5 were combined to form OL-6. Brine solution (prepared from demineralised water (300.0 L) and sodium chloride (12.0 Kg) at 25 to 30° C. The reaction mixture was stirred at 30 to 35° C. for 30 minutes and allowed to settle at 30 to 35° C. for 30 minutes. The layers were separated (aqueous layer (AL-5) and organic layer (OL-7)) and OL-7 was kept aside. OL-7 was dried over anhydrous Na₂SO₄ (9.0 Kg) and the anhydrous Na₂SO₄ was washed with toluene (30.0 L) at 25 to 30° C. The solvent was distilled under vacuum at below 40 to 45° C. until 5% remained. Methanol (60.0 L) was charged to the reaction mixture at 40 to 45° C. and down to 60 L of reaction mass.

Methanol (120.0 L) was charged to the reaction mixture at 40 to 45° C. and the reaction mixture was cooled to 5 to 10° C. and maintained at 5 to 10° C. for 30 minutes. The solid product was filtered, washed with cooled methanol (30.0 L), and dried in a hot air oven at 40 to 45° C. for 6 hours to afford 38.87 Kg of a mixture of 8-nitro-1,2,3,5,6,7-hexahydro-s-indacen-1-one (11a) and 4-nitro-1,2,3,5,6,7-hexahydro-s-indacen-1-one (11b)

Combined Yield (11a+11b): 62.24%

Weight ratio (11a:11b): 9:1

HPLC purity: 95.9%

Moisture content: 0.19%

¹H NMR: (500 MHz, CDCl₃): δ 7.44 (S, 1H), 2.21 (m, 2H), 2.78 (t, 2H), 3.02 (m, 4H), 3.13 (t, 2H)

### Reaction Scheme 2—Step (d)

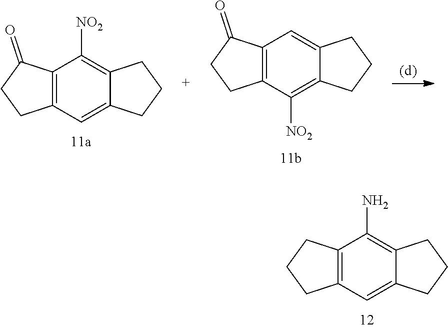 [SMILES: O=C1CCc2cc3c(c([N+](=O)[O-])c21)CCC3 ; O=C1CCc2c1cc1c(c2[N+](=O)[O-])CCC1 ; Nc1c2c(cc3c1CCC3)CCC2]

A mixture of 8-nitro-1,2,3,5,6,7-hexahydro-s-indacen-1-one (11a) and 4-nitro-1,2,3,5,6,7-hexahydro-s-indacen-1-one (11b) (9:1 ratio; 27.0 Kg) at 25 to 30° C. was charged into a 600 L clean and dry pressure reactor.

Methanol (270 L) was charged at 25 to 30° C. Methane sulfonic acid (14.3 Kg) was slowly charged at 25 to 30° C. and the reaction mixture was maintained for 30 minutes. 15% Pd(OH)₂ slurry (60% wet)*² was added. *2: To prepare the 15% Pd(OH)₂ slurry, 20% Pd(OH)₂ on carbon (60% wet; 4.05 Kg) was added to methanol (27 L).

The reaction mixture was degassed under vacuum and filled with an argon atmosphere (0.5 Kg) three times. The reaction mixture was degassed under vacuum and filled with a hydrogen atmosphere (0.5 Kg) three times. Then the reaction mixture was stirred under hydrogen pressure (100 Psi) at room temperature for 32 hours. The temperature was gradually raised up to 55° C. The absence of 8-nitro-1,2,3,5,6,7-hexahydro-s-indacen-1-one (11a) and 4-nitro-1,2,3,5,6,7-hexahydro-s-indacen-1-one (11b) was confirmed by HPLC (Limit: 1.0%).

After completion of the reaction, the reaction mixture was cooled to 25 to 30° C. The reaction mixture was degassed under vacuum and filled with nitrogen atmosphere (0.5 Kg) three times.

The reaction mixture was filtered through a candy filter to remove Pd(OH)₂, followed by a micro filter and the bed was washed with methanol (54 L). 95% of the solvent was distilled off under vacuum at below 45 to 50° C. Demineralised water (135 L) was charged into the reaction mixture at 25 to 30° C. and maintained for 30 minutes. The reaction mixture was cooled to 5-10° C. The pH was adjusted to about 9-10 with 2 N aqueous NaOH solution (prepared from NaOH (6.48 Kg) and demineralised water (81 L)) and the reaction mixture was stirred for 30 minutes. Then toluene (135 L) was charged to the reaction mixture and the reaction mixture was stirred for 30 minutes. The reaction mixture was stirred for a further 30 minutes, whilst bringing the temperature up to 25 to 30° C. The reaction mixture was allowed to settle for 30 minutes, whilst the temperature was maintained at 25 to 30° C.

The reaction mixture was filtered through a Celite® bed (prepared with Celite® (5.4 Kg) and toluene (13.5 L). The Celite® bed was washed with toluene (54 L).

The layers were separated (aqueous layer (AL-1) and organic layer (OL-1)) and OL-1 was kept aside. Toluene (54 L) was added to AL-1 at 25 to 30° C. The reaction mixture was stirred at 25 to 30° C. for 30 minutes and allowed to settle at 25 to 30° C. for 30 minutes. The layers were separated (aqueous layer (AL-2) and organic layer (OL-2)) and AL-2 was kept aside. Toluene (54 L) was added to AL-1 at 25 to 30° C. A brine solution (prepared with demineralised water (135 L) and sodium chloride (54 Kg)) was charged to the combined organic layers (OL-1 and OL-2) at 25 to 30° C. The reaction mixture was stirred at 25 to 30° C. for 30 minutes and allowed to settle at 25 to 30° C. for 30 minutes.

The layers were separated (aqueous layer (AL-3) and organic layer (OL-3)) and AL-3 was kept aside. Charcoal (1.3 Kg) was added to OL-3 and the temperature was raised to 35-40° C. and maintained at 35 to 40° C. for 30 minutes. The reaction mixture was filtered through a Celite® bed (prepared with Celite® (5-4 Kg) and toluene (54 L)) at 35 to 40° C. The Celite® bed was washed with toluene (54 L). The organic layer was dried over anhydrous Na₂SO₄ (13.5 Kg). The Na₂SO₄ was washed with toluene (27 L).

The solvent was distilled under vacuum at below 35 to 40° C. until 5% remained. Methanol (40.5 L) was charged to the reaction mixture at 35 to 40° C. and distilled until 5% remained. Methanol (97.2 L) and water (10.8 L) were charged to the reaction mixture at 35 to 40° C. The reaction mixture was heated to 50 to 55° C., stirred for 1 hour at 50 to 55° C., slowly cooled to 0 to 5° C. and maintained at 0 to 5° C. for 30 minutes.

The solid product was filtered and washed with cold methanol (13.5 L), and dried in a hot air oven at 40 to 45° C. for 6 hours to afford 11.3 Kg of crude 1,2,3,5,6,7-hexahydro-s-indacen-4-amine (12).

Yield: 41.85%

HPLC purity: 98.10%

Moisture content: 0.10

¹H NMR: (400 MHz, DMSO-d₆): δ 6.38 (S, 1H), 4.45 (S, 2H), 2.75 (t, 4H), 2.58 (t, 4H), 1.98 (t, 4H).

### Purification (A) of 1,2,3,5,6,7-hexahydro-s-indacen-4-amine (12)

Crude 1,2,3,5,6,7-hexahydro-s-indacen-4-amine (12) (54.5 Kg) was charged at 25 to 30° C. into a 250 L clean and dry reactor. Toluene (27.2 L) was charged at 25 to 30° C. and the reaction mixture was stirred at 25 to 30° C. for 30 minutes. Methanol (163 L) was charged to the reaction mixture at 25 to 30° C. The reaction mixture was stirred at 25 to 30° C. for 30 minutes, cooled to −5 to 0° C., and stirred at −5 to 0° C. for 30 minutes. The solid product was filtered, washed with cold methanol (54.5 L), and dried at 40 to 45° C. for 6 hours to afford 40.5 Kg of purified 1,2,3,5,6,7-hexahydro-s-indacen-4-amine (12).

Yield: 74.31%

HPLC purity: 99.5%

Moisture content: 0.3%

¹H NMR: (400 MHz, DMSO-d₆): δ 6.33 (s, 1H), 4.53 (s, 2H), 2.72 (t, 4H), 2.57 (t, 4H), 1.98 (t, 4H).

Crop Purification (B) of 1,2,3,5,6,7-hexahydro-s-indacen-4-amine (12)

The filtered mother liquors from five batches of reaction scheme 2, step (d) were combined and concentrated to afford crude 1,2,3,5,6,7-hexahydro-s-indacen-4-amine (12) (25 Kg) and purified through a 100-200 mesh silica gel column. The column was eluted with 5 to 10% ethyl acetate (42 L) in hexane (658 L).

The pure fractions were concentrated under reduced pressure (600 mm of Hg) at 40 to 45° C. to afford crude 1,2,3,5,6,7-hexahydro-s-indacen-4-amine (12) (15 Kg).

Toluene (7.5 L) was added at 25 to 30° C. and the reaction mixture was stirred for 30 minutes at 25 to 30° C. Methanol (45 L) was added at 25 to 30° C. and the reaction mixture was stirred for 30 minutes at 25 to 30° C. The reaction mixture was cooled to −5 to 10° C. and stirred for 30 minutes. Purity was checked using HPLC (Limit 98%, Single max purity: NMT: 1%).

The solid was filtered, washed with cold methanol (15 L) and dried at 40 to 45° C. in vacuum tray drier for 6 hours to afford 10.2 Kg of purified 1,2,3,5,6,7-hexahydro-s-indacen-4-amine (12).

Yield: 9.36%

HPLC purity: 99.3%

Moisture content: 0.12%

¹H NMR: (400 MHz, DMSO-d₆): δ 6.33 (S, 1H), 4.51 (S, 2H), 2.72 (t, 4H), 2.59 (t, 4H), 1.99 (t, 4H).

Combined yield of five batches of reaction scheme 2, step (d) including purification (A) and crop purification (B): 46.56%

### Reaction Scheme 2—Step (e)

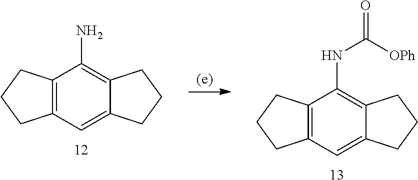

1,2,3,5,6,7-hexahydro-s-indacen-4-amine (12)(7.50 Kg) was charged to a clean and dry reactor. THF (60.05 Kg) was added to the reactor and the temperature was adjusted to between 0 and 10° C. to form a clear brown solution. N,N′-diisopropylethylamine (6.66 Kg) dissolved in THF (6.78 Kg) was charged to the reactor whilst maintaining the temperature between 0 and 10° C. (line rinse with THF (6.78 Kg) at 0 to 10° C.). The temperature was maintained at 0 to 5° C.

Phenyl chloroformate (7.44 Kg) dissolved in THF (6.74 Kg) was charged to the reactor over a minimum of 1 hour whilst maintaining the temperature between 0 and 10° C. to form a slurry (line rinse with THF (6.66 Kg) at 0 to 10° C.). The temperature of the reaction mixture was raised to between 15 and 25° C. and stirred until complete. Completion was measured by ¹H NMR analysis. Pass criterion≤1.0 mol % 1,2,3,5,6,7-hexahydro-s-indacen-4-amine (12).

The temperature of the reaction mixture was increased to between 3° and 40° C. The reaction mixture was concentrated under reduced pressure to about 37.5 L. Absolute ethanol (31.50 Kg) was charged to the reaction mixture at between 3° and 40° C. The reaction mixture was concentrated under reduced pressure to about 37.5 L. Absolute ethanol (29.60 Kg) was charged to the reaction mixture at between 3° and 40° C. The reaction mixture was concentrated under reduced pressure to about 37.5 L. Absolute ethanol (29.74 Kg) was charged to the reaction mixture at between 3° and 40° C. The reaction mixture was concentrated under reduced pressure to about 37.5 L. Absolute ethanol charging and concentrating was repeated until sample of the reaction mixture passes analysis by ¹H NMR. Pass criterion≤0.5% w/w THF relative to product.

Absolute ethanol (30.12 Kg) was charged to the reaction mixture at between 15 and 40° C. The reaction mixture was cooled to between 0 and 5° C. and stirred for 45 to 90 minutes. The solid was filtered on a 20 μm filter cloth at 0 to 5° C. The solid was washed with absolute ethanol (11.72 Kg and 12.00 Kg) at 0 to 5° C. and sucked down on the filter for 30 to 90 minutes under nitrogen purge.

The solid was identified and analysed by HPLC. Pass criterion≤0.5% DIPEA·HCl relative to product. The solid was dried under vacuum at up to 50° C. under a flow of nitrogen until the ethanol content was ≤0.5% w/w, to afford 11.78 Kg of 4-(phenoxycarbonylamino)-1,2,3,5,6,7-hexahydro-s-indacene (13).

Yield: 93%

HPLC purity: 99.6%

### 1-Ethyl-N-((1,2,3,5,6,7-hexahydro-s-indacen-4-yl)carbamoyl)piperidine-4-sulfonamide (potassium salt) (14)

1-Ethyl-N-((1,2,3,5,6,7-hexahydro-s-indacen-4-yl)carbamoyl)piperidine-4-sulfonamide (potassium salt) (14) was prepared according to the reaction sequence illustrated in reaction scheme 3:

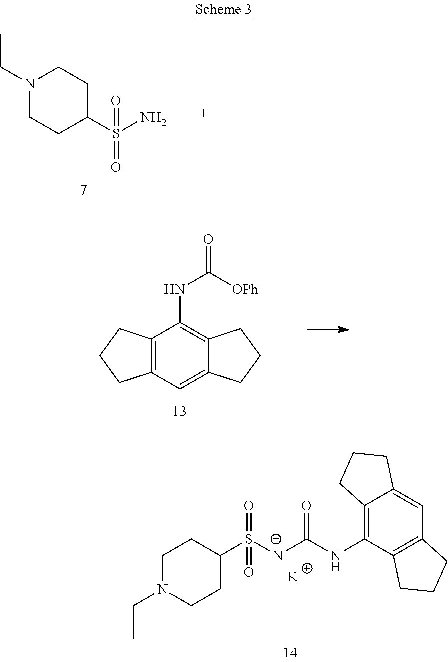 [SMILES: CCN1CCC(S(N)(=O)=O)CC1 ; O=C(Nc1c2c(cc3c1CCC3)CCC2)Oc1ccccc1 ; CCN1CCC(S(=O)(=O)[N-]C(=O)Nc2c3c(cc4c2CCC4)CCC3)CC1 ; [K+]]

1-ethyl-4-piperidinesulfonamide (7) (7.85 Kg) was charged to a vessel. Dimethyl sulfoxide (33.5 Kg) was charged to the vessel and the mixture was adjusted to 20 to 25° C. The mixture was stirred for at least 60 minutes (target 60 to 90 minutes) at 20 to 25° C. until full solution was obtained. Potassium tert-butoxide (5.1 Kg) was charged in at least six portions to the vessel over at least 60 minutes (target 60 to 90 minutes) maintaining the temperature at 20 to 30° C. (target 20 to 25° C.). The mixture was adjusted to 20 to 25° C. and stirred for at least 30 minutes (target 30 to 60 minutes) at to 25° C.

4-(phenoxycarbonylamino)-1,2,3,5,6,7-hexahydro-s-indacene (13) (12.55 Kg) was charged in at least six portions to the vessel over at least 30 minutes (target 30 to 90 minutes) maintaining the temperature at 20 to 30° C. The reaction mixture was stirred at 20 to 30° C. for at least 60 minutes or until reaction completed. A sample was analysed for completion by ¹H NMR. Pass criterion≤5.0 mol % 1-ethyl-4-piperidinesulfonamide (7), taking a consecutive passing sample.

The reaction mixture was weighed in a separate container and then transferred back to the vessel using a line rinse of dimethyl sulfoxide (17.2 Kg). The mixture was stirred and adjusted to 20 to 25° C. The water content was analysed by KF.

Acetonitrile (62.0 Kg) was charged to the vessel over at least 30 minutes maintaining the temperature at 20 to 25° C. Water (3.00 Kg) was charged to the vessel over 2-3 hours maintaining the temperature at 20 to 25° C. Acetonitrile (19.4 Kg) was charged to the vessel maintaining the temperature at 20 to 25° C. The mixture was stirred for at least 1 hour (target 1 to 3 hours) at 20 to 25° C. The mixture was cooled to 0 to 5° C. over at least 1 hour (target 1 to 2 hours), stirred for at least 1 hour (target 1 to 4 hours) at 0 to 5° C., filtered over 1 to 2 μm cloth at 0 to 5° C. and the filter cake was washed with pre-mixed (6:13:0.4) dimethyl sulfoxide/acetonitrile/water (5.34 Kg:8.32 Kg:0.31 Kg) at 0 to 5° C.

The solid was dried under vacuum for ca. 2 hours until suitable for handling and the filter cake was analysed for water content by KF. Pass criterion≤5.5% w/w.

The filter cake was slurry washed with acetonitrile (62.3 Kg) at 15 to 25° C. for 30 to 60 minutes before filtering at 15 to 25° C. The filter cake was washed with acetonitrile (19.6 Kg) at 15 to 25° C. The filter cake was slurry washed with acetonitrile (61.9 Kg) at 15 to 25° C. for at least 30 minutes (target 30 to 60 minutes) before filtering at 15 to 25° C. The filter cake was washed with acetonitrile (19.2 Kg) at 15 to 25° C. The filter cake was slurry washed with acetonitrile (62.0 Kg) at 15 to 25° C. for at least 30 minutes (target to 60 minutes) before filtering at 15 to 25° C. The filter cake was washed with acetonitrile (18.5 Kg) at 15 to 25° C.

The solid was dried at up to 50° C. under a flow of nitrogen and analysed by KF for residual water content. Pass criterion≤2.8% w/w water. The solid was analysed for residual DMSO levels by ¹H NMR. Pass criterion≤12.2% w/w DMSO. The solid was analysed for residual acetonitrile levels by ¹H NMR. Pass criterion≤2.0% w/w MeCN. The dried weight of the crude solid was measured, identified and analysed using ¹H NMR spectroscopy and HPLC. 13.95 Kg of crude 1-ethyl-N-((1,2,3,5,6,7-hexahydro-s-indacen-4-yl)-carbamoyl)piperidine-4-sulfonamide (potassium salt) (14) was obtained.

Yield: 80%

NMR purity: 97.3%

### Purification of 1-ethyl-N-((1,2,3,5,6,7-hexahydro-s-indacen-4-yl)-carbamoyl)-piperidine-4-sulfonamide (Potassium Salt) (14)

Crude 1-ethyl-N-((1,2,3,5,6,7-hexahydro-s-indacen-4-yl)carbamoyl)piperidine-4-sulfonamide (potassium salt) (14) (14.71 Kg) was charged to a reaction vessel. Methanol (116.4 Kg) was charged to the vessel, the temperature was adjusted to 15 to 25° C. as required with stirring for 10 to 20 minutes (until a homogeneous cloudy solution with no lumps of solid present was formed). The solution was filtered through a 1 μm filter at to 25° C. The filter was washed with methanol (11.3 Kg) at 15 to 25° C. The solution was concentrated to ca. 44 L at 25 to 35° C. Acetonitrile (116.6 Kg) was charged to the mixture and the solution was concentrated to ca. 74 L at 25 to 35° C. Acetonitrile (58.7 Kg) was charged to the mixture and the mixture was concentrated to ca. 74 L at s 35° C. The mixture was analysed for residual methanol content by ¹H NMR. Pass criterion s 3.0% w/w methanol.

Acetonitrile (58.8 Kg) was charged to the vessel and the temperature was adjusted to 15 to 25° C. The slurry was aged for at least 1 hour (target 1 to 2 hours) at 15 to 25° C. and then filtered over 20 μm cloth at 15 to 25° C. The filter cake was twice washed with acetonitrile (23.9 Kg, 23.6 Kg) at 15 to 25° C.

The damp filter cake was analysed for residual phenol by HPLC. Pass criterion: ≤0.20% area phenol. The solid was dried at up to 50° C. under a flow of nitrogen for at least 2 hours and analysed for residual water content using KF. Pass criterion≤2.0% w/w. Drying continued whilst the sample was being analysed.

The solid was analysed for residual acetonitrile by ¹H NMR. Pass criterion≤0.2% w/w MeCN. The solid was analysed for residual DMSO by ¹H NMR. Pass criterion≤0.4% w/w DMSO. The solid was analysed for residual solvent levels by GC. Pass criteria≤3750 ppm DMSO, ≤2250 ppm MeOH and ≤308 ppm MeCN. 14.42 Kg of purified 1-ethyl-N-((1,2,3,5,6,7-hexahydro-s-indacen-4-yl)-carbamoyl)piperidine-4-sulfonamide (potassium salt) (14) was obtained.

Yield: 98%

HPLC purity: 99.5%

1. A process of preparing a thiourea adduct (I) or a salt thereof, wherein the process comprises converting a N-protected-4-derivatised piperidine (H) to the thiourea adduct (I) or the salt thereof: 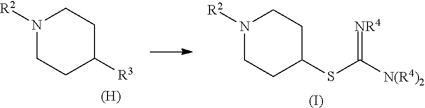
wherein:
R² is a nitrogen protecting group;
R³ is a leaving group; and
each R⁴ is independently selected from hydrogen r-n a C₁-C₂₀ hydrocarbyl group, wherein
each C₁-C₂₀ hydrocarbyl group is straight-chained or branched, or is or includes one or more cyclic groups, wherein
each C₁-C₂₀ hydrocarbyl group ais optionally substituted with one or more groups selected from oxo (═0) and halo, and wherein
each C₁-C₂₀ hydrocarbyl group optionally includes one or more heteroatoms, each independently selected from N, O and S, or wherein
any two or more R⁴, together with the atom or atoms to which they are attached, form a 3- to 16-membered heterocyclic group, wherein
the heterocyclic group is monocyclic, bicyclic or tricyclic, and wherein
the heterocyclic group is optionally substituted with one or more groups selected from halo and R⁴⁰, wherein
each R⁴⁰ is independently selected from —CN, —OH, —NH₂, oxo (═O), ═NH and C₁-C₆ hydrocarbyl, wherein
each C₁-C₆ hydrocarbyl group is straight-chained or branched, or is or includes one or more cyclic groups, wherein
each C₁-C₆ hydrocarbyl group is optionally substituted with one or more halo, and wherein
each C₁-C₆ hydrocarbyl group optionally includes one or more heteroatoms independently selected from N, O and S.

2. The process of claim 1, wherein R² is a nitrogen protecting group removeable by catalytic hydrogenolysis.

3. The process of claim 1, wherein R² is —CH₂R²⁰ or —COOCH₂R²⁰, wherein
R²⁰ is an aryl or heteroaryl group, wherein
the aryl or heteroaryl group is monocyclic, bicyclic or tricyclic, wherein
the aryl or heteroaryl group is optionally substituted with one or more substituents independently selected from halo, —CN, —OH, —NO₂, —NH₂, —R²¹, —OR²¹, —NHR²¹, —N(R²¹)₂ and —N(O)(R²¹)₂, wherein
each R²¹ is independently selected from a C₁-C₄ alkyl, C₁-C₄ haloalkyl, C₃-C₄ cycloalkyl and C₃-C₄ halocycloalkyl group, or
any two R²¹ directly attached to the same nitrogen atom together form a C₂-C₅ alkylene or C₂-C₅ haloalkylene group, and wherein
R²⁰ contains from 1 to 20 carbon atoms.

4. The process of claim 1, wherein R² is —COOCH₂Ph.

5. The process of claim 1, wherein R³ is a sulfonate leaving group.

6. The process of claim 1, wherein each R⁴ is independently selected from hydrogen, C₁-C₆ alkyl, C₃-C₆ cycloalkyl, or any two R⁴ may together form a C₂-C₆ alkylene group, wherein any C₁-C₆ alkyl, C₃-C₆ cycloalkyl or C₂-C₆ alkylene are each independently optionally substituted with fluoro.

7. The process of claim 1, wherein each R⁴ is hydrogen.

8. The process of claim 1, further comprising contacting the N-protected-4-derivatised piperidine (H) with reagent (I-X): 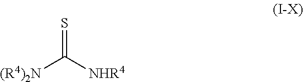
optionally in the presence of a base and/or a solvent.

9. The process of claim 1, further comprising contacting benzyl 4-((methylsulfonyl)oxy)piperidine-1-carboxylate (H′) with reagent (I-Xb) in a solvent to obtain benzyl 4-(carbamimidoylthio)-piperidine-1-carboxylate (I′) or a salt thereof:

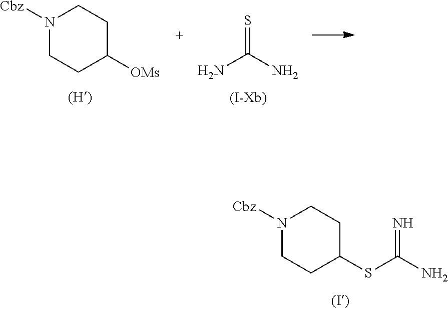

10. The process of claim 1, wherein the thiourea adduct (I) or (I′) is obtained as a methanesulfonic acid salt.

11. The process of claim 1, wherein the N-protected-4-derivatised piperidine (H) or (H′) is obtained by e process comprising:
(i) converting 4-hydroxy piperidine (F) to a N-protected-4-hydroxy piperidine (G): 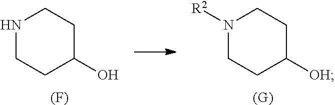
and
(ii) converting the N-protected-4-hydroxy piperidine (G) to the N-protected-4-derivatised piperidine (H):

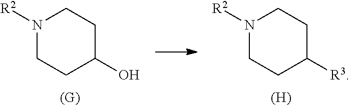

12. A process of preparing a N-protected-4-(halosulfonyl)-piperidine (J) or a salt thereof, wherein the process comprises converting a thiourea adduct (I) to the N-protected-4-(halosulfonyl)-piperidine (J) or the salt thereof: 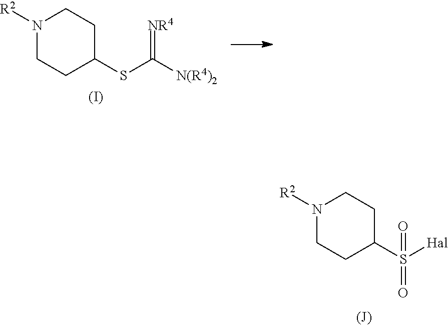
wherein:
R² is a nitrogen protecting group;
each R⁴ is independently selected from hydrogen or a C₁-C₂₀ hydrocarbyl group, wherein
each C₁-C₂₀ hydrocarbyl group is straight-chained or branched, or is or includes one or more cyclic groups, wherein
each C₁-C₂₀ hydrocarbyl group is optionally substituted with one or more oxo (═O) and/or one or more halo, and wherein
each C₁-C₂₀ hydrocarbyl group optionally includes one or more heteroatoms independently selected from N, O and S, or wherein
any two or more R⁴, together with the atom or atoms to which they are attached, form a 3- to 16-membered heterocyclic group, wherein
the heterocyclic group is monocyclic, bicyclic or tricyclic, and wherein
the heterocyclic group is optionally substituted with one or more groups selected from halo and/or R⁵⁰, wherein
each R⁴⁰ is independently selected from —CN, —OH, —NH₂, oxo (═O), ═NH and C₁-C₆ hydrocarbyl, wherein
each C₁-C₆ hydrocarbyl is straight-chained, branched, or is or includes one or more cyclic groups, wherein
each C₁-C₆ hydrocarbyl group is optionally substituted with one or more halo, and wherein
each C₁-C₆ hydrocarbyl group optionally includes one or more heteroatoms independently selected from N, O and S; and
Hal is Cl or Br.

13. The process of claim 12, wherein R² is a nitrogen protecting group removeable by catalytic hydrogenolysis.

14. The process of claim 12, wherein
R² is —CH₂R²⁰ or —COOCH₂R²⁰, wherein
R²⁰ is an aryl or heteroaryl group, wherein
the aryl or heteroaryl group is monocyclic, bicyclic or tricyclic, wherein the aryl or heteroaryl group are each interpedently optionally substituted with one or more substituents selected from halo, —CN, —OH, —NO₂, —NH₂, —R²¹, —OR²¹, —NHR²¹, —N(R²¹)₂ and —N(O)(R²¹)₂, wherein
each R²¹ is independently selected from —C₁-C₄ alkyl, C₁-C₄ haloalkyl, C₃-C₄ cycloalkyl and C₃-C₄ halocycloalkyl, or
any two R²¹ directly attached to the same nitrogen atom together form a C₂-C₅ alkylene or C₂-C₅ haloalkylene, and wherein
R²⁰ contains from 1 to 20 carbon atoms.

15. The process of claim 12, wherein R⁴ is independently selected from hydrogen, C₁-C₆ alkyl, C₃-C₆ cycloalkyl, or any two R⁴ may together form a C₂-C₆ alkylene, wherein any C₁-C₆ alkyl, C₃-C₆ cycloalkyl or C₂-C₆ alkylene are each independently optionally substituted with fluoro.

16. The process of claim 12, wherein each R⁴ is independently selected from hydrogen, C₁-C₆ alkyl, C₃-C₆ cycloalkyl, or any two R⁴ may together form a C₂-C₆ alkylene, wherein any C₁-C₆ alkyl, C₃-C₆ cycloalkyl or C₂-C₆ alkylene are each optionally substituted with fluoro.

17. The process of claim 12, wherein each R⁴ is hydrogen.

18. The process of claim 12, wherein Hal is Cl.

19. The process of claim 12, further comprising the step of contacting the thiourea adduct (I) with a halogenating agent, to form the N-protected-4-(halosulfonyl)-piperidine (J) or the salt thereof.

20. The process of claim 19, wherein the thiourea adduct (I) is contacted with the halogenating agent in the presence of a carboxylic acid, water, and optionally a second acid selected from HCl or HBr.

21. The process of claim 19, wherein the process comprises contacting benzyl 4-(carbamimidoylthio)-piperidine-1-carboxylate (I′) with a chlorinating agent to obtain benzyl 4-(chlorosulfonyl)-1-piperidinecarboxylate (J′) or a salt thereof:

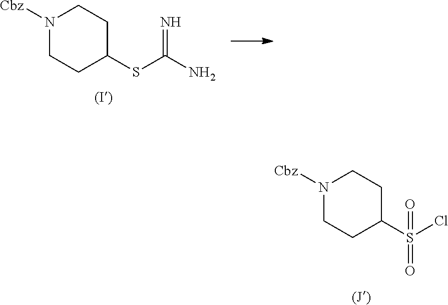 [SMILES: N=C(N)SC1CCN(C(=O)OCc2ccccc2)CC1 ; O=C(OCc1ccccc1)N1CCC(S(=O)(=O)Cl)CC1]

22. The process of claim 12, wherein the thiourea adduct (I) or (I′) is obtained by a process according to claim 1.

23. The process of claim 12, further comprising:
(v) converting the N-protected-4-(halosulfonyl)-piperidine (J) or (J′) to a N-protected-4-piperidinesulfonamide (K): 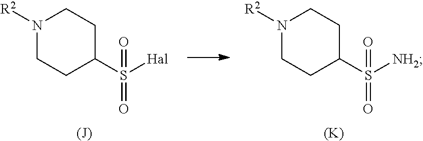
and
(vi) optionally converting the N-protected-4-piperidinesulfonamide (K) to 1-ethyl-4-piperidinesulfonamide (A):

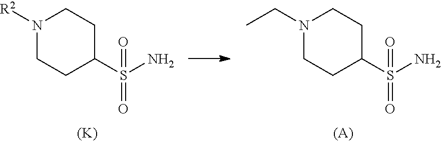

24. A process comprising one or more steps selected from:
(i) converting 4-hydroxy piperidine (F) to a N-protected-4-hydroxy piperidine (G): 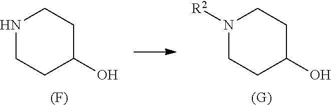
wherein the conversion is performed in a biphasic solvent system;
(ii) converting a N-protected-4-hydroxy piperidine (G) to a N-protected-4-derivatised piperidine (H): 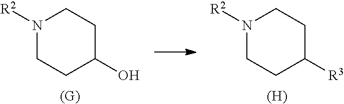
wherein the conversion is performed in the presence of a non-polar solvent;
(iii) converting a N-protected-4-derivatised piperidine (H) to a thiourea adduct (I): 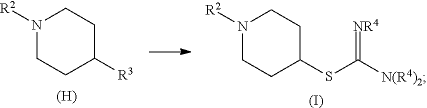
(iv) converting a thiourea adduct (I) to a N-protected-4-(halosulfonyl)-piperidine (J): 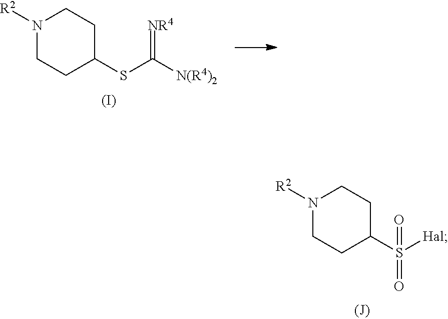
(v) converting a N-protected-4-(halosulfonyl)-piperidine (J) to a N-protected-4-piperidinesulfonamide (K): 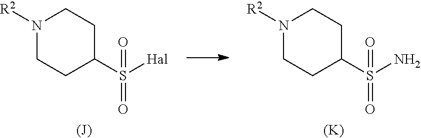
wherein the conversion comprises (1) forming a solution of ammonia in a solvent, and (2) adding the N-protected-4-(halosulfonyl)-piperidine (J) to the solution formed in (1); and
(vi) converting a N-protected-4-piperidinesulfonamide (K) to 1-ethyl-4-piperidinesulfonamide (A): 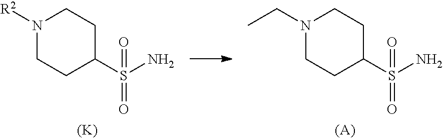
wherein the conversion is performed in the presence of a C₃-C₅ alcohol;
and wherein:
R² is a nitrogen protecting group;
R³ is a leaving group;
each R⁴ is independently selected from hydrogen and a C₁-C₂₀ hydrocarbyl group, wherein
each C₁-C₂₀ hydrocarbyl group is straight-chained, branched, or is or includes one or more cyclic groups, wherein
each C₁-C₂₀ hydrocarbyl group mas optionally substituted with one or more oxo (═O) and/or halo, and wherein
each C₁-C₂₀ hydrocarbyl group optionally includes one or more heteroatoms independently selected from N, O and S, or wherein
any two or more R⁴, together with the atom or atoms to which they are attached, form a 3- to 16-membered heterocyclic group, wherein
the heterocyclic group is monocyclic, bicyclic or tricyclic, and wherein
the heterocyclic group is optionally substituted with one or more halo and/or R⁴⁰, wherein
each R⁴⁰ is independently selected from a —CN, —OH, —NH₂, oxo (═O), ═NH and C₁-C₆ hydrocarbyl group, wherein
each C₁-C₆ hydrocarbyl group is straight-chained or branched, or is or includes one or more cyclic groups, wherein
each C₁-C₆ hydrocarbyl group is optionally substituted with one or more halo, and wherein
each C₁-C₆ hydrocarbyl group optionally includes one or more heteroatoms independently selected from N, O and S; and
Hal is Cl or Br.

25. The process of claim 24, further comprising contacting the 1-ethyl-4-piperidinesulfonamide (A) with a 1,2,3,5,6,7-hexahydro-s-indacene derivative (B) in the presence of a solvent to obtain 1-ethyl-N-((1,2,3,5,6,7-hexahydro-s-indacen-4-yl)carbamoyl)piperidine-4-sulfonamide (C) or a salt thereof: 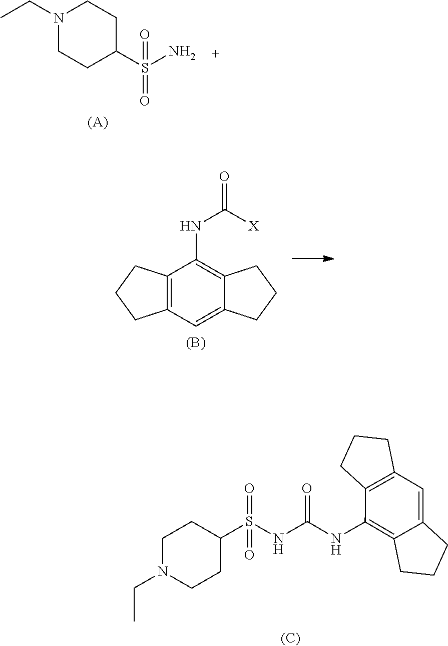
wherein X is a leaving group.

26. 1-ethyl-N-((1,2,3,5,6,7-hexahydro-s-indacen-4-yl)carbamoyl)piperidine-4-sulfonamide or a salt thereof, prepared by the process of claim 25.

27. A thiourea adduct (I) or a salt thereof: 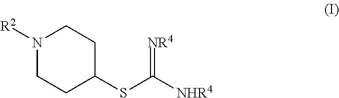
wherein:
R² is a nitrogen protecting group; and
each R⁴ is independently selected from hydrogen and a C₁-C₂₀ hydrocarbyl group, wherein
each C₁-C₂₀ hydrocarbyl group is straight-chained, branched, or is or includes one or more cyclic groups, wherein
each C₁-C₂₀ hydrocarbyl group is optionally substituted with one or more oxo (═O) and/or halo, and wherein
each C₁-C₂₀ hydrocarbyl group optionally includes one or more heteroatoms independently selected from N, O and S, or wherein
any two or more R⁴, together with the atom or atoms to which they are attached, form a 3- to 16-membered heterocyclic group, wherein
the heterocyclic group is monocyclic, bicyclic or tricyclic, and wherein
the heterocyclic group is optionally substituted with one or more halo and/or R⁴⁰, wherein
each R⁴⁰ is independently selected from a —CN, —OH, —NH₂, oxo (═O), ═NH and C₁-C₆ hydrocarbyl, wherein
each C₁-C₆ hydrocarbyl group is straight-chained, branched, or is or includes one or more cyclic groups, wherein
each C₁-C₆ hydrocarbyl group is optionally substituted with one or more halo, and wherein
each C₁-C₆ hydrocarbyl group optionally includes one or more heteroatoms independently selected from N, O and S.

28. The thiourea adduct (I) or the salt thereof of claim 27, wherein R² is —COOCH₂Ph and each R⁴ is hydrogen.

29. A compound selected from the group consisting of:
(i) an N-protected-4-(halosulfonyl)-piperidine (J) or a salt thereof: 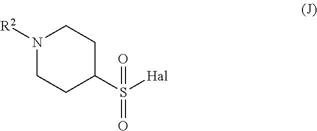
wherein R² is a nitrogen protecting group and Hal is Cl or Br; and
(ii) an N-protected-4-piperidinesulfonamide (K) or a salt thereof: 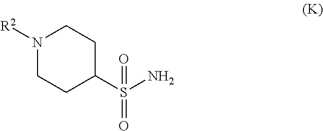
wherein R² is a nitrogen protecting group.

30. A compound of claim 29, wherein R² is —COOCH₂Ph and Hal is Cl.

31. A compound of claim 29, wherein:
(i) the N-protected-4-(halosulfonyl)-piperidine (J) or the salt thereof is in solid form; and/or
(ii) the N-protected-4-(halosulfonyl)-piperidine (J) or the salt thereof has an, HPLC purity of ≥90%; and/or
(iii) the N-protected-4-piperidinesulfonamide (K) or the salt thereof has a HPLC purity of ≥96.2%.

32. The process of claim 5, wherein R³ is —OMs.

33. The process of claim 19, wherein the halogenating agent is N-chlorosuccinimide.

34. The process of claim 20, wherein the carboxylic acid is acetic acid.
